(19) United States
(12) Patent Application Publication
(10) Pub. No.: US 2022/0185832 A1
(43) Pub. Date: Jun. 16, 2022

(54) CD73 INHIBITORS

(71) Applicant: BioArdis LLC, San Diego, CA (US)

(72) Inventors: Simon BAILEY, San Diego, CA (US); Dianjun CHEN, Beijing (CN); Jianxia FENG, Beijing (CN); Frank KAYSER, San Rafael, CA (US); Chong LIU, Beijing (CN); Hongbin YUAN, Shanghai (CN)

(21) Appl. No.: 17/603,550

(22) PCT Filed: Apr. 3, 2020
(86) PCT No.: PCT/CN2020/083200

§ 371 Date: Oct. 13, 2021

(30) Foreign Application Priority Data
Apr. 15, 2019 (CN) PCT/CN2019/082651

(51) Int. Cl. C07F 9/6558

(52) U.S. Cl. CPC C07F 9/65586

(57) ABSTRACT

Disclosed are compounds that arc inhibitors of CD73 and arc useful in treating CD73-associated diseases or conditions, and compositions containing the compounds.

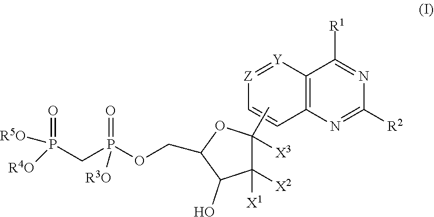

### CROSS REFERENCE TO RELATED APPLICATION

This application claims priority to International Patent Application No. PCT/CN2019/082651, filed on Apr. 15, 2019, the content of which is incorporated herein by reference in its entirety.

### FIELD

The present disclosure relates generally to compounds that are inhibitors of CD73 and are useful in treating CD73-associated diseases or conditions. Compositions containing the compounds of the present disclosure are also provided.

### BACKGROUND

CD73 is a 70-kDa glycosylphosphatidylinositol (GPI)-anchored protein normally expressed on endothelial cells and subsets of hematopoietic cells. CD73 is up-regulated by hypoxia-inducible factor (HIF)-la and after exposure to type I interferons. In steady state, CD73 regulates vascular barrier function, restricts lymphocyte migration to draining lymph nodes, and stimulates mucosal hydration.

CD73 expression on tumor cells has been reported in several types of cancer, including bladder cancer, leukemia, glioma, glioblastoma, melanoma, ovarian cancer, thyroid cancer, esophageal cancer, prostate cancer, and breast cancer. (Stagg, et al., Proc. Natl. Acad. Sci. USA 107(4): 1547-1552). Notably, CD73 expression has been associated with a prometastatic phenotype in melanoma and breast cancer.

There is still a need for new CD73 inhibitors. In this regard, the compounds provided herein address the need.

### BRIEF SUMMARY

In one aspect, provided herein is a compound of formula (I):

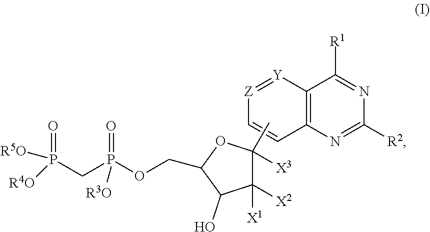

or a stereoisomer, tautomer, prodrug, or a pharmaceutically acceptable salt of any of the foregoing, wherein Y, Z, X¹, X², X³, and R¹-R⁵ are as described herein.

In another aspect, provided herein is a composition comprising a compound of formula (I), or a stereoisomer, tautomer, prodrug, or a pharmaceutically acceptable salt of any of the foregoing and a pharmaceutically acceptable excipient.

In another aspect, provided herein is a kit comprising a compound of formula (I), or a stereoisomer, tautomer, prodrug, or a pharmaceutically acceptable salt of any of the foregoing. In some embodiments, provided herein is a medicament comprising a compound of formula (I), or a stereoisomer, tautomer, prodrug, or a pharmaceutically acceptable salt of any of the foregoing.

In another aspect, provided herein is a method of treating a treating a disease mediated by CD73 in an individual in need thereof, comprising administering to the individual a therapeutically effective amount of a compound of formula (I), or a stereoisomer, tautomer, prodrug, or a pharmaceutically acceptable salt of any of the foregoing. In some embodiments, the disease is cancer. In some embodiments, the disease is bladder cancer, leukemia, glioma, glioblastoma, melanoma, ovarian cancer, thyroid cancer, esophageal cancer, prostate cancer, lung cancer, colorectal cancer, pancreatic cancer, skin cancer, liver cancer, gastric cancer, head & neck cancer, or breast cancer.

In another aspect, provided herein is a method of inhibition CD73, comprising contacting CD73 with a compound of formula (I), or a stereoisomer, tautomer, prodrug, or a pharmaceutically acceptable salt of any of the foregoing.

In another aspect, provided herein is a compound of formula (I), or a stereoisomer, tautomer, prodrug, or a pharmaceutically acceptable salt of any of the foregoing, in the manufacture of a medicament for use in therapy.

In another aspect, provided herein are methods of preparing a compound of formula (I), or a stereoisomer, tautomer, prodrug, or a pharmaceutically acceptable salt of any of the foregoing, according to the procedures detailed herein.

### DETAILED DESCRIPTION

Described herein are compounds, including therapeutic agents, that can inhibit CD73. These compounds could be used in the prevention and/or treatment of certain pathological conditions as described herein.

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

“Alkyl” as used herein refers to and includes, unless otherwise stated, a saturated linear (i.e., unbranched) or branched univalent hydrocarbon chain or combination thereof, having the number of carbon atoms designated (i.e., C₁₋₁₀ means one to ten carbon atoms). Particular alkyl groups are those having 1 to 20 carbon atoms (a “C₁₋₂₀ alkyl”), having 1 to 10 carbon atoms (a “C₁₋₁₀ alkyl”), having 6 to 10 carbon atoms (a “C₆₋₁₀ alkyl”), having 1 to 6 carbon atoms (a “C₁₋₆ alkyl”), having 2 to 6 carbon atoms (a “C₂₋₆ alkyl”), or having 1 to 4 carbon atoms (a “C₁₋₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkoxy” refers to an —O-alkyl. Examples of alkoxy include, but are not limited to, methoxy, ethoxy, n-propoxy, isopropoxy, n-butoxy, iso-butoxy, sec-butoxy, and tert-butoxy.

“Alkenyl” as used herein refers to and includes, unless otherwise stated, an unsaturated linear (i.e., unbranched) or branched univalent hydrocarbon chain or combination thereof, having at least one site of olefinic unsaturation (i.e., having at least one moiety of the formula C═C) and having the number of carbon atoms designated (i.e., C₂₋₁₀ means two to ten carbon atoms). An alkenyl group may have “cis” or “trans” configurations, or alternatively have “E” or “Z” configurations. Particular alkenyl groups are those having 2 to 20 carbon atoms (a “C₂₋₂₀ alkenyl”), having 6 to 10 carbon atoms (a “C₆₋₁₀ alkenyl”), having 2 to 8 carbon atoms (a “C₂₋₈ alkenyl”), having 2 to 6 carbon atoms (a “C₂₋₆ alkenyl”), or having 2 to 4 carbon atoms (a “C₂₋₄ alkenyl”). Examples of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, pent-1-enyl, pent-2-enyl, hex-1-enyl, hex-2-enyl, hex-3-enyl, and the like.

“Alkynyl” as used herein refers to and includes, unless otherwise stated, an unsaturated linear (i.e., unbranched) or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Particular alkynyl groups are those having 2 to 20 carbon atoms (a ^{“}C₂₋₂₀ alkynyl”), having 6 to 10 carbon atoms (a “C₆₋₁₀ alkynyl”), having 2 to 8 carbon atoms (a “C₂₋₈ alkynyl”), having 2 to 6 carbon atoms (a “C₂₋₆ alkynyl”), or having 2 to 4 carbon atoms (a “C₂₋₄ alkynyl”). Examples of alkynyl group include, but are not limited to, groups such as ethynyl (or acetylenyl), prop-1-ynyl, prop-2-ynyl (or propargyl), but-1-ynyl, but-2-ynyl, but-3-ynyl, and the like.

“Cycloalkyl” as used herein refers to and includes, unless otherwise stated, cyclic univalent nonaromatic hydrocarbon structures, which may be fully saturated, mono- or polyunsaturated, but which are non-aromatic, having the number of carbon atoms designated (i.e., C₃₋₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃₋₈ cycloalkyl”), having 3 to 6 carbon atoms (a “C₃₋₆ cycloalkyl”), or having from 3 to 4 annular carbon atoms (a “C₃₋₄ cycloalkyl”). Examples of cycloalkyl include, but are not limited to, cyclopropyl, cyclobutyl, cyclopentyl, cyclohexyl, cycloheptyl, norbornyl, and the like. A cycloalkyl group may be fused with aryl, heteroaryl, or heterocyclyl. In one variation, a cycloalkyl group having more than one ring where at least one ring is aryl, heteroaryl, or heterocyclyl is connected to the parent structure at an atom in the nonaromatic hydrocarbon cyclic group.

“Aryl” or “Ar” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular carbon atoms (a “C₆₋₁₄ aryl”). An aryl group may be fused with heteroaryl, cycloalkyl, or heterocyclyl. In one variation, an aryl group having more than one ring where at least one ring is heteroaryl, cycloalkyl, or heterocyclyl is connected to the parent structure at an atom in the aromatic carbocyclic group.

“Heteroaryl” as used herein refers to an unsaturated aromatic cyclic group having from 1 to 14 annular carbon atoms and at least one annular heteroatom, including but not limited to heteroatoms such as nitrogen, oxygen, and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5 to 14-membered rings having 1 to 12 annular carbon atoms and 1 to 6 annular heteroatoms independently selected from nitrogen, oxygen, and sulfur, 5 to 10-membered rings having 1 to 8 annular carbon atoms and 1 to 4 annular heteroatoms independently selected from nitrogen, oxygen, and sulfur, or 5, 6 or 7-membered rings having 1 to 5 annular carbon atoms and 1 to 4 annular heteroatoms independently selected from nitrogen, oxygen, and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen, and sulfur. A heteroaryl group may be fused with aryl, cycloalkyl, or heterocyclyl. In one variation, a heteroaryl group having more than one ring where at least one ring is aryl, cycloalkyl, or heterocyclyl is connected to the parent structure at an atom in the aromatic cyclic group having at least one annular heteroatom. A heteroaryl group may be connected to the parent structure at a ring carbon atom or a ring heteroatom.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof, but excludes heteroaryl. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur. A heterocyclyl group may be fused with aryl, cycloalkyl, or heteroaryl. In one variation, a heterocyclyl group having more than one ring where at least one ring is aryl, cycloalkyl, or heteroaryl is connected to the parent structure at an atom in the non-aromatic cyclic group having at least one heteroatom.

“Halo” or “halogen” refers to elements of the Group 17 series having atomic number 9 to 85. Preferred halo groups include the radicals of fluorine, chlorine, bromine and iodine. A haloalkyl is an alkyl group that is substituted with one or more halogens. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl.

“Carbonyl” refers to the group C═O.

“Acyl” refers to —C(═O)R where R is an aliphatic group, preferably a C₁₋₆ moiety. The term “aliphatic” refers to saturated and unsaturated straight chained, branched chained, or cyclic hydrocarbons. Examples of aliphatic groups include, but are not limited to, C₁₋₆ alkyl, C₂₋₆ alkenyl, C₂₋₆ alkynyl, or C₃₋₆ cycloalkyl.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods of the present disclosure contemplate any one or more of these aspects of treatment.

As used herein, the term “effective amount” intends such amount of a compound described herein which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

A “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the present disclosure in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The term “prodrug” as used herein refers to a compound which provides an active compound following administration to the individual in which it is used, by a chemical and/or biological process in vivo (e.g., by hydrolysis and/or an enzymatic conversion). The prodrug itself may be active, or it may be relatively inactive, then transformed into a more active compound. This disclosure embraces prodrugs of the compounds described herein.

When a moiety is indicated as substituted by “at least one” substituent, this also encompasses the disclosure of exactly one sub stituent.

### Compounds

In one aspect, provided is a compound of formula (I):

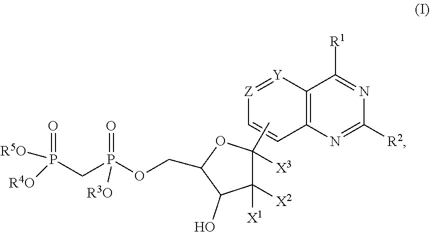

or a stereoisomer, tautomer, prodrug, or a pharmaceutically acceptable salt of any of the foregoing, wherein:
X¹, X², and X³ are each independently H, —CN, C₁₋₆ alkyl, —OR′, or halogen, wherein R′ is H, C₁₋₆ alkyl, C₃₋₁₂ cycloalkyl, 3- to 12-membered heterocyclyl, 5- to 10-membered heteroaryl, or C₆₋₁₄ aryl;
Y is —CR^{Y}— or N, wherein R^{Y} is H, C₁₋₆ alkyl, or halogen;
Z is —CR^{Z}— or N, wherein R^{Z} is H, C₁₋₆ alkyl, or halogen;
R¹ is —NR^{1a}R^{1b} or —OR^{1a}, wherein R^{1a} and R^{1b} are each independently H, C₁₋₆ alkyl, C₃₋₁₂ cycloalkyl, 3- to 12-membered heterocyclyl, 5- to 10-membered heteroaryl, or C₆₋₁₄ aryl, wherein the C₁₋₆ alkyl, C₃₋₁₂ cycloalkyl, 3- to 12-membered heterocyclyl, 5- to 10-membered heteroaryl, and C₆₋₁₄ aryl are each independently optionally substituted with R⁶, or

R^{1a} and R^{1b} are taken together with the nitrogen atom to which they attach to form a 3- to 12-membered heterocyclyl, which is optionally substituted with R⁶;
R² is H, C₁₋₆ alkyl, C₂₋₆ alkenyl, C₂₋₆ alkynyl, halogen, —CN, —OR^{2a}, —SR^{2a}, —NR^{2a}R^{2b}, —OC(O)R^{2a}, —NR^{2a}C(O)R^{2b}, —NR^{2a}C(O)OR^{2b}, —NR^{2a}S(O)R^{2b}, —NR^{2a}S(O)₂R^{2b}, —C(O)NR^{2a}R^{2b}, —C(O)NR^{2a}S(O)₂R^{2b}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently optionally substituted with R⁷, and wherein:

R^{2a} and R^{2b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{2a} and R^{2b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
R³, R⁴, and R⁵ are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl;
each R⁶ is independently oxo, C₁₋₆ alkyl, C₂₋₆ alkenyl, C₂₋₆ alkynyl, halogen, —CN, —OR^{6a}, —SR^{6a}, —NR^{6a}R^{6b}, —NO₂, —C═NH(OR^{6a}), —C(O)R^{6a}, —OC(O)R^{6a}, —C(O)OR^{6a}, —C(O)NR^{6a}R^{6b}, —OC(O)NR^{6a}R^{6b}, —NR^{6a}C(O)R^{6b}, —NR^{6a}C(O)OR^{6b}, —S(O)R^{6a}, —S(O)₂R^{6a}, —NR^{6a}S(O)R^{6b}, —C(O)NR^{6a}S(O)R^{6b}, —NR^{6a}S(O)₂R^{6b}, —C(O)NR^{6a}S(O)₂R^{6b}, —S(O)NR^{6a}R^{6b}, —S(O)₂NR^{6a}R^{6b}, —P(O)(OR^{6a}) (OR^{6b}), C₃₋₆ cycloalkyl, 3- to 12-membered heterocyclyl, 5- to 10-membered heteroaryl, or C₆₋₁₄ aryl, wherein the C₃₋₆ cycloalkyl, 3- to 12-membered heterocyclyl, 5- to 10-membered heteroaryl, and C₆₋₁₄ aryl are each independently optionally substituted with C₁₋₆ alkyl, C₂₋₆ alkenyl, C₂₋₆ alkynyl, halogen, hydroxyl, C₁₋₆ alkoxy, or —CN, and wherein:

R^{6a} and R^{6b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{6a} and R^{6b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
each R⁷ is independently oxo, C₁₋₆ alkyl, C₂₋₆ alkenyl, C₂₋₆ alkynyl, halogen, —CN, —SR^{7a}, —NR^{7a}R^{7b}, —NO₂, —C═NH(OR^{7a}), —C(O)R^{7a}, —OC(O)R^{7a}, —C(O)OR^{7a}, —C(O)NR^{7a}R^{7b}, —OC(O)NR^{7a}R^{7b}, —NR^{7a}C(O)R^{7b}, —NR^{7a}C(O)OR^{7b}, —S(O)R^{7a}, —S(O)₂R^{7a}, —NR^{7a}S(O)R^{7b}, —C(O)NR^{7a}S(O)R^{7b}, —NR^{7a}S(O)₂R^{7b}, —C(O)NR^{7a}S(O)₂R^{7b}, —S(O)NR^{7a}R^{7b}, —S(O)₂NR^{7a}R^{7b}, —P(O)(OR^{7a}) (OR^{7b}), C₃₋₆ cycloalkyl, 3- to 12-membered heterocyclyl, 5- to 10-membered heteroaryl, or C₆₋₁₄ aryl, wherein:

R^{7a} and R^{7b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{7a} and R^{7b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN.

In some embodiments, provided is a compound of formula (I):

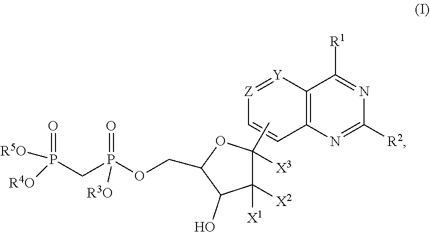

or a stereoisomer, tautomer, prodrug, or a pharmaceutically acceptable salt of any of the foregoing, wherein:
X¹, X², and X³ are each independently H, —CN, C₁₋₆ alkyl, —OR′, or halogen, wherein R′ is H, C₁₋₆ alkyl, C₃₋₁₂ cycloalkyl, 3- to 12-membered heterocyclyl, 5- to 10-membered heteroaryl, or C₆₋₁₄ aryl;
Y is —CR^{Y}— or N, wherein R^{Y} is H, C₁₋₆ alkyl, or halogen;
Z is —CR^{Z}— or N, wherein R^{Z} is H, C₁₋₆ alkyl, or halogen;
R¹ is —NR^{1a}R^{1b} or —OR^{1a}, wherein R^{1a} and R^{1b} are each independently H, C₁₋₆ alkyl, C₃₋₁₂ cycloalkyl, 3- to 12-membered heterocyclyl, 5- to 10-membered heteroaryl, or C₆₋₁₄ aryl, wherein the C₁₋₆ alkyl, C₃₋₁₂ cycloalkyl, 3- to 12-membered heterocyclyl, 5- to 10-membered heteroaryl, and C₆₋₁₄ aryl are each independently optionally substituted with R⁶, or

R^{1a} and R^{1b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
R² is H, C₁₋₆ alkyl, C₂₋₆ alkenyl, C₂₋₆ alkynyl, halogen, —CN, —OR^{2a}, —SR^{2a}, —NR^{2a}R^{2b}, —OC(O)R^{2a}, —NR^{2a}C(O)R^{2b}, —NR^{2a}C(O)OR^{2b}, —NR^{2a}S(O)R^{2b}, —NR^{2a}S(O)₂R^{2b}, —C(O)NR^{2a}R^{2b}, —C(O)NR^{2a}S(O)₂R^{2b}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently optionally substituted with R⁷, and wherein:

R^{2a} and R^{2b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{2a} and R^{2b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
R³, R⁴, and R⁵ are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl;
each R⁶ is independently oxo, C₁₋₆ alkyl, C₂₋₆ alkenyl, C₂₋₆ alkynyl, halogen, —CN, —OR^{6a}, —SR^{6a}, —NR^{6a}R^{6b}, —NO₂, —C═NH(OR^{6a}), —C(O)R^{6a}, —OC(O)R^{6a}, —C(O)OR^{6a}, —C(O)NR^{6a}R^{6b}, —OC(O)NR^{6a}R^{6b}, —NR^{6a}C(O)R^{6b}, —NR^{6a}C(O)OR^{6b}, —S(O)R^{6a}, —S(O)₂R^{6a}, —NR^{6a}S(O)R^{6b}, —C(O)NR^{6a}S(O)R^{6b}, —NR^{6a}S(O)₂R^{6b}, —C(O)NR^{6a}S(O)₂R^{6b}, —S(O)NR^{6a}R^{6b}, —S(O)₂NR^{6a}R^{6b}, —P(O)(OR^{6a}) (OR^{6b}), C₃₋₆ cycloalkyl, 3- to 12-membered heterocyclyl, 5- to 10-membered heteroaryl, or C₆₋₁₄ aryl, wherein the C₃₋₆ cycloalkyl, 3- to 12-membered heterocyclyl, 5- to 10-membered heteroaryl, and C₆₋₁₄ aryl are each independently optionally substituted with C₁₋₆ alkyl, C₂₋₆ alkenyl, C₂₋₆ alkynyl, halogen, hydroxyl, C₁₋₆ alkoxy, or —CN, and wherein:

R^{6a} and R^{6b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{6a} and R^{6b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
each R⁷ is independently oxo, C₁₋₆ alkyl, C₂₋₆ alkenyl, C₂₋₆ alkynyl, halogen, —CN, —SR^{7a}, —NR^{7a}R^{7b}, —NO₂, —C═NH(OR^{7a}), —C(O)R^{7a}, —OC(O)R^{7a}, —C(O)OR^{7a}, —C(O)NR^{7a}R^{7b}, —OC(O)NR^{7a}R^{7b}, —NR^{7a}C(O)R^{7b}, —NR^{7a}C(O)OR^{7b}, —S(O)R^{7a}, —S(O)₂R^{7a}, —NR^{7a}S(O)R^{7b}, —C(O)NR^{7a}S(O)R^{7b}, —NR^{7a}S(O)₂R^{7b}, —C(O)NR^{7a}S(O)₂R^{7b}, —S(O)NR^{7a}R^{7b}, —S(O)₂NR^{7a}R^{7b}, —P(O)(OR^{7a}) (OR^{7b}), C₃₋₆ cycloalkyl, 3- to 12-membered heterocyclyl, 5- to 10-membered heteroaryl, or C₆₋₁₄ aryl, wherein:

R^{7a} and R^{7b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{7a} and R^{7b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN.

In some embodiments, the compound of formula (I) is of formula (II), or a stereoisomer, tautomer, prodrug, or a pharmaceutically acceptable salt of any of the foregoing,

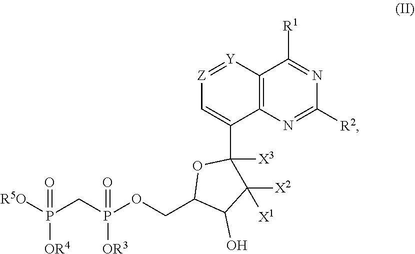

wherein Z, Y, X¹, X², X³, and R¹-R⁵ are as defined herein for any embodiment of a compound of formula (I).

In some embodiments, the compound of formula (I) is of formula (III), or a stereoisomer, tautomer, prodrug, or a pharmaceutically acceptable salt of any of the foregoing,

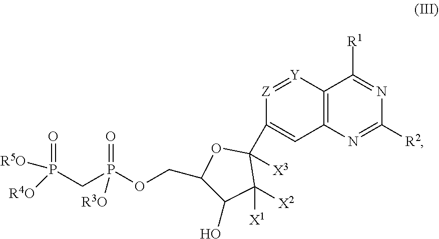

wherein Z, Y, X¹, X², X³, and R¹-R⁵ are as defined herein for any embodiment of a compound of formula (I).

In some embodiments of a compound of formula (I), or any related formula, or a stereoisomer, tautomer, prodrug, or a pharmaceutically acceptable salt of any of the foregoing, Y is N. In some embodiments, Y is —CR^{Y}—.

In some embodiments of a compound of formula (I), or any related formula, or a stereoisomer, tautomer, prodrug, or a pharmaceutically acceptable salt of any of the foregoing, Z is N. In some embodiments, Z is —CR^{Z}—.

In some embodiments of a compound of formula (I), or any related formula, or a stereoisomer, tautomer, prodrug, or a pharmaceutically acceptable salt of any of the foregoing, Y is N and Z is —CR^{Z}—. In some embodiments, Y is —CR^{Y}— and Z is N. In some embodiments, Y is —CR^{Y}— and Z is —CR^{Z}—.

In some embodiments, the compound of formula (II) is of formula (IV), or a stereoisomer, tautomer, prodrug, or a pharmaceutically acceptable salt of any of the foregoing,

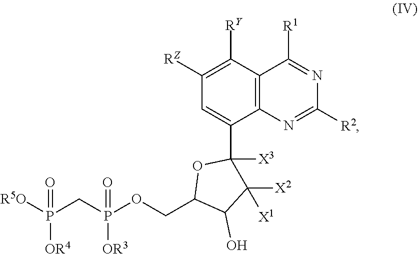

wherein X¹, X², X³, R^{Y}, R^{Z}, and R¹-R⁵ are as defined herein for any embodiment of a compound of formula (I).

In some embodiments, the compound of formula (III) is of formula (V), or a stereoisomer, tautomer, prodrug, or a pharmaceutically acceptable salt of any of the foregoing,

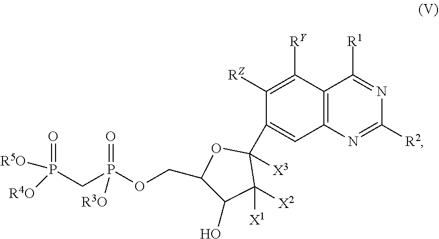

wherein X¹, X², X³, R^{Y}, R^{Z}, and R¹-R⁵ are as defined herein for any embodiment of a compound of formula (I).

In some embodiments, the compound of formula (I) is of any of the formulae provided below, or a stereoisomer, tautomer, prodrug, or a pharmaceutically acceptable salt of any of the foregoing.

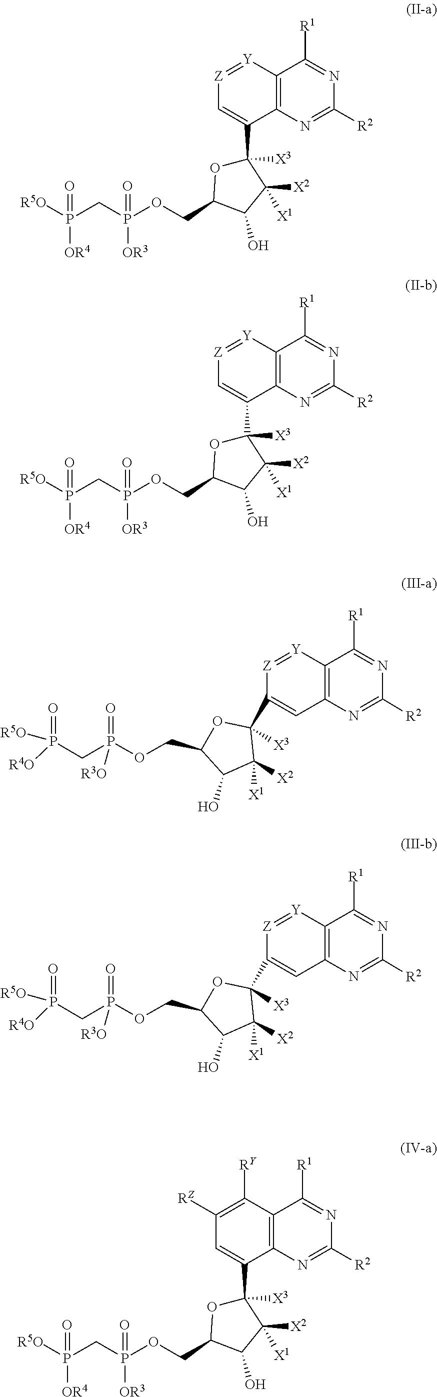

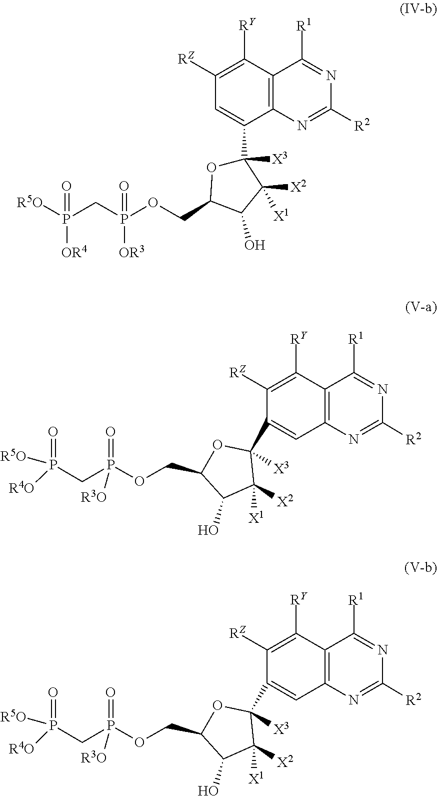

In some embodiments of a compound of formula (I), or any related formula, or a stereoisomer, tautomer, prodrug, or a pharmaceutically acceptable salt of any of the foregoing, R³ is H. In some embodiments, R³ is C₁₋₆ alkyl, such as methyl, ethyl, n-propyl, isopropyl, n-butyl, t-butyl, isobutyl, or sec-butyl. In some embodiments, R³ is C₂₋₆ alkenyl, such as ethenyl, prop-1-enyl, prop-2-enyl, 2-methylprop-1-enyl, but-1-enyl, but-2-enyl, or but-3-enyl. In some embodiments, R³ is C₂₋₆ alkynyl, such as ethynyl, prop-1-ynyl, prop-2-ynyl, but-1-ynyl, but-2-ynyl, or but-3-ynyl. In some embodiments, R³ is C₃₋₁₂ cycloalkyl. In some embodiments, R³ is C₃₋₆ cycloalkyl, such as cyclopropyl, cyclobutyl, cyclopentyl, or cyclohexyl. In some embodiments, R³ is C₆₋₁₄ aryl, such as phenyl or naphthyl. In some embodiments, R³ is phenyl. In some embodiments, R³ is 5- to 10-membered heteroaryl. In some embodiments, R³ is 5- or 6-membered heteroaryl, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R³ is 3- to 12-membered heterocyclyl. In some embodiments, R³ is 5- or 6-membered heterocyclyl, such as tetrahydrofuranyl, pyrrolidinyl, piperidinyl, piperazinyl, morpholinyl, or thiomorpholinyl.

In some embodiments of a compound of formula (I), or any related formula, or a stereoisomer, tautomer, prodrug, or a pharmaceutically acceptable salt of any of the foregoing, R⁴ is H. In some embodiments, R⁴ is C₁₋₆ alkyl, such as methyl, ethyl, n-propyl, isopropyl, n-butyl, t-butyl, isobutyl, or sec-butyl. In some embodiments, R⁴ is C₂₋₆ alkenyl, such as ethenyl, prop-1-enyl, prop-2-enyl, 2-methylprop-1-enyl, but-l-enyl, but-2-enyl, or but-3-enyl. In some embodiments, R⁴ is C₂₋₆ alkynyl, such as ethynyl, prop-1-ynyl, prop-2-ynyl, but-1-ynyl, but-2-ynyl, or but-3-ynyl. In some embodiments, R⁴ is C₃₋₁₂ cycloalkyl. In some embodiments, R⁴ is C₃₋₆ cycloalkyl, such as cyclopropyl, cyclobutyl, cyclopentyl, or cyclohexyl. In some embodiments, R⁴ is C₆₋₁₄ aryl, such as phenyl or naphthyl. In some embodiments, R⁴ is phenyl. In some embodiments, R⁴ is 5- to 10-membered heteroaryl. In some embodiments, R⁴ is 5- or 6-membered heteroaryl, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R⁴ is 3- to 12-membered heterocyclyl. In some embodiments, R⁴ is 5- or 6-membered heterocyclyl, such as tetrahydrofuranyl, pyrrolidinyl, piperidinyl, piperazinyl, morpholinyl, or thiomorpholinyl.

In some embodiments of a compound of formula (I), or any related formula, or a stereoisomer, tautomer, prodrug, or a pharmaceutically acceptable salt of any of the foregoing, R⁵ is H. In some embodiments, R⁵ is C₁₋₆ alkyl, such as methyl, ethyl, n-propyl, isopropyl, n-butyl, t-butyl, isobutyl, or sec-butyl. In some embodiments, R⁵ is C₂₋₆ alkenyl, such as ethenyl, prop-1-enyl, prop-2-enyl, 2-methylprop-1-enyl, but-1-enyl, but-2-enyl, or but-3-enyl. In some embodiments, R⁵ is C₂₋₆ alkynyl, such as ethynyl, prop-1-ynyl, prop-2-ynyl, but-1-ynyl, but-2-ynyl, or but-3-ynyl. In some embodiments, R⁵ is C₃₋₁₂ cycloalkyl. In some embodiments, R⁵ is C₃₋₆ cycloalkyl, such as cyclopropyl, cyclobutyl, cyclopentyl, or cyclohexyl. In some embodiments, R⁵ is C₆₋₁₄ aryl, such as phenyl or naphthyl. In some embodiments, R⁵ is phenyl. In some embodiments, R⁵ is 5- to 10-membered heteroaryl. In some embodiments, R⁵ is 5- or 6-membered heteroaryl, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R⁵ is 3- to 12-membered heterocyclyl. In some embodiments, R⁵ is 5- or 6-membered heterocyclyl, such as tetrahydrofuranyl, pyrrolidinyl, piperidinyl, piperazinyl, morpholinyl, or thiomorpholinyl.

In some embodiments of a compound of formula (I), or any related formula, or a stereoisomer, tautomer, prodrug, or a pharmaceutically acceptable salt of any of the foregoing, R³ is H; R⁴ is H; and R⁵ is H.

In some embodiments, the compound of formula (I) is of any of the formulae provided below, or a stereoisomer, tautomer, prodrug, or a pharmaceutically acceptable salt of any of the foregoing.

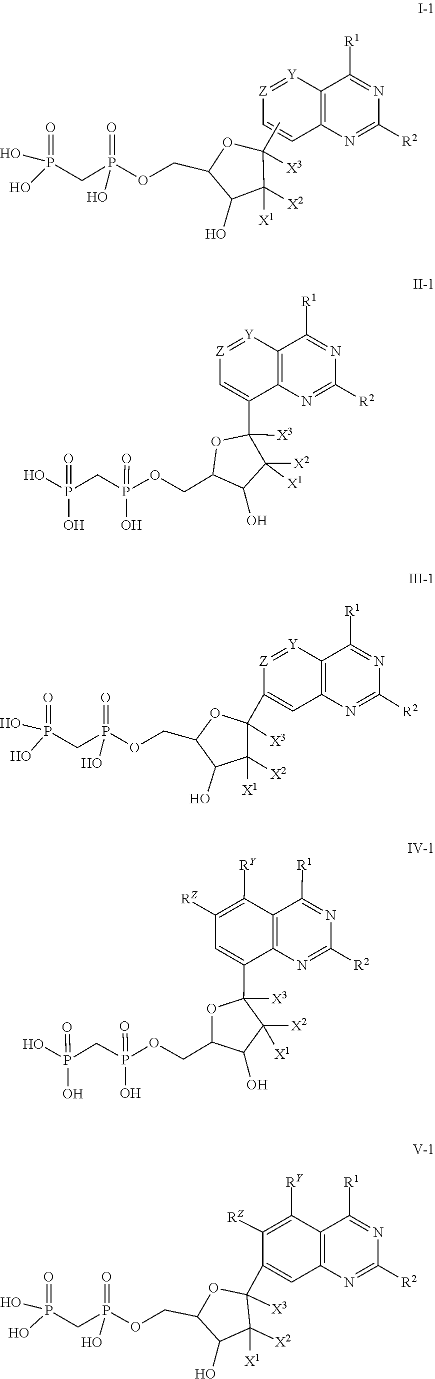

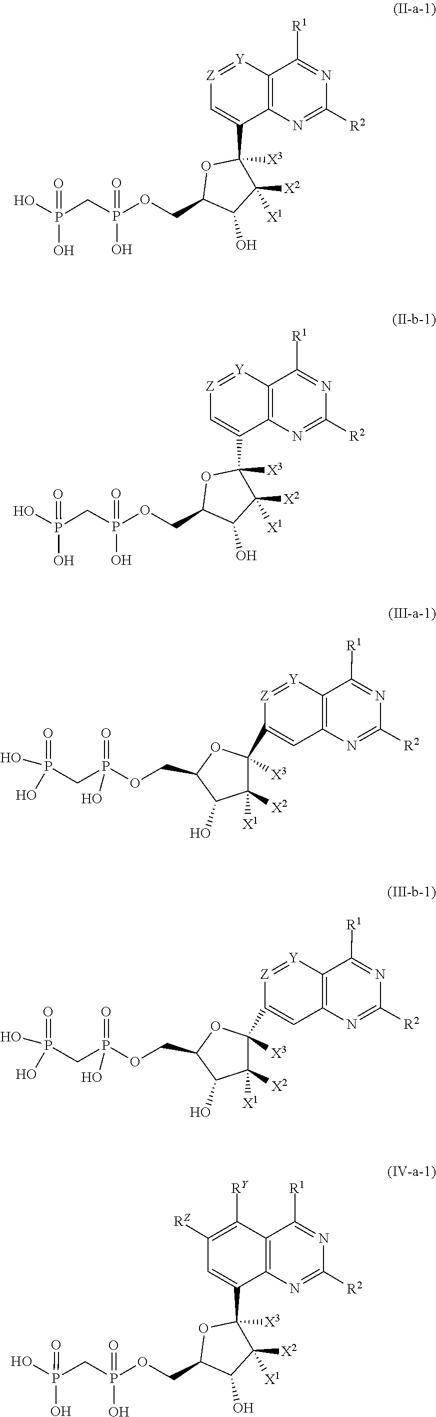

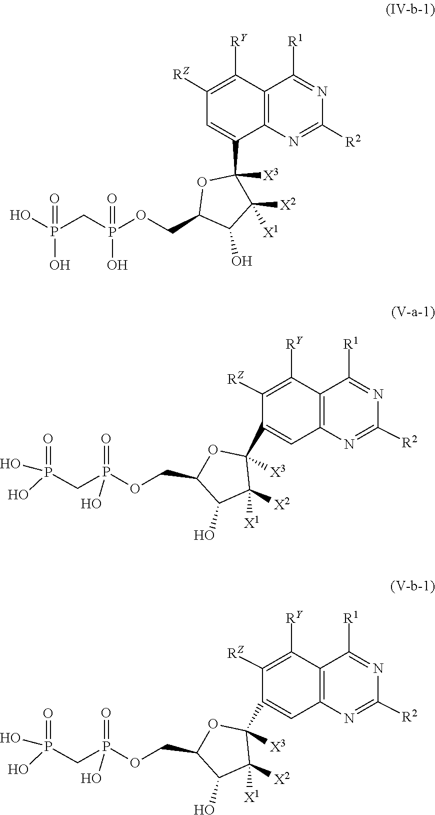

In some embodiments of a compound of formula (I), or any related formula, or a stereoisomer, tautomer, prodrug, or a pharmaceutically acceptable salt of any of the foregoing, R^{Y} is H. In some embodiments, R^{Y} is C₁₋₆ alkyl, such as methyl, ethyl, n-propyl, isopropyl, n-butyl, t-butyl, isobutyl, or sec-butyl. In some embodiments, R^{Y} is halogen such as fluoro, chloro, or bromo. In some embodiments, R^{Y} is H or C₁₋₆ alkyl. In some embodiments, R^{Y} is H or halogen.

In some embodiments of a compound of formula (I), or any related formula, or a stereoisomer, tautomer, or a pharmaceutically acceptable salt of any of the foregoing, R^{Z} is H. In some embodiments, R^{Z} is C₁₋₆ alkyl, such as methyl, ethyl, n-propyl, isopropyl, n-butyl, t-butyl, isobutyl, or sec-butyl. In some embodiments, R^{Z} is halogen such as fluoro, chloro, or bromo. In some embodiments, R^{Z} is chloro. In some embodiments, R^{Z} is H or C₁₋₆ alkyl. In some embodiments, R^{Z} is H or halogen.

In some embodiments of a compound of formula (I), or any related formula, or a stereoisomer, tautomer, prodrug, or a pharmaceutically acceptable salt of any of the foregoing, R^{Y} is H and R^{Z} is H or halogen. In some embodiments, R^{Y} is H and R^{Z} is H.

In some embodiments of a compound of formula (I), or any related formula, or a stereoisomer, tautomer, prodrug, or a pharmaceutically acceptable salt of any of the foregoing, X¹ is H. In some embodiments, X¹ is —CN. In some embodiments, X¹ is C₁₋₆ alkyl such as methyl, ethyl, n-propyl, isopropyl, n-butyl, t-butyl, isobutyl, or sec-butyl. In some embodiments, X¹ is —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X¹ is —OH. In some embodiments, X¹ is halogen, such as fluoro, chloro, or bromo. In some embodiments, X¹ is H or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X¹ is H or halogen. In some embodiments, X¹ is H or —OH.

In some embodiments of a compound of formula (I), or any related formula, or a stereoisomer, tautomer, prodrug, or a pharmaceutically acceptable salt of any of the foregoing, X² is H. In some embodiments, X² is —CN. In some embodiments, X² is C₁₋₆ alkyl such as methyl, ethyl, n-propyl, isopropyl, n-butyl, t-butyl, isobutyl, or sec-butyl. In some embodiments, X² is —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X² is —OH. In some embodiments, X² is halogen such as fluoro, chloro, or bromo. In some embodiments, X² is fluoro. In some embodiments, X² is H or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X² is H, halogen, or C₁₋₆ alkyl. In some embodiments, X² is H, fluoro, or methyl. In some embodiments, X² is fluoro. In some embodiments, X² is methyl.

In some embodiments of a compound of formula (I), or any related formula, or a stereoisomer, tautomer, prodrug, or a pharmaceutically acceptable salt of any of the foregoing, X¹ is H or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl; and X² is H, halogen, or C₁₋₆ alkyl. In some embodiments, X¹ is H or —OH; and X² is H, halogen, or C₁₋₆ alkyl. In some embodiments, X¹ is H or —OH; and X² is H, fluoro, or methyl.

In some embodiments of a compound of formula (I), or any related formula, or a stereoisomer, tautomer, prodrug, or a pharmaceutically acceptable salt of any of the foregoing, X³ is H. In some embodiments, X³ is —CN. In some embodiments, X³ is C₁₋₆ alkyl such as methyl, ethyl, n-propyl, isopropyl, n-butyl, t-butyl, isobutyl, or sec-butyl. In some embodiments, X³ is —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X³ is —OH. In some embodiments, X³ is halogen such as fluoro, chloro, or bromo. In some embodiments, X³ is H or —CN.

In some embodiments of a compound of formula (I), or any related formula,or a stereoisomer, tautomer, prodrug, or a pharmaceutically acceptable salt of any of the foregoing, X¹ is H or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl; X² is H, halogen, or C₁₋₆ alkyl; and X³ is H or —CN. In some embodiments, X¹ is H or —OH; X² is H, halogen, or C₁₋₆ alkyl; and X³ is H or —CN. In some embodiments, X¹ is H or —OH; X² is H, fluoro, or methyl; and X³ is H or —CN.

In some embodiments of a compound of formula (I), or any related formula, or a stereoisomer, tautomer, or a pharmaceutically acceptable salt of any of the foregoing, R¹ is —NR^{1a}R^{1b}. In some embodiments, R¹ is —OR^{1a}.

In some embodiments of a compound of formula (I), or any related formula, or a stereoisomer, tautomer, prodrug, or a pharmaceutically acceptable salt of any of the foregoing, R^{1a} is H. In some embodiments, R^{1a} is C₁₋₆ alkyl optionally substituted with R⁶, such as methyl, ethyl, n-propyl, isopropyl, n-butyl, t-butyl, isobutyl, or sec-butyl, each of which is independently optionally substituted with R⁶. In some embodiments, R^{1a} is C₁₋₆ alkyl such as methyl, ethyl, n-propyl, isopropyl, n-butyl, t-butyl, isobutyl, or sec-butyl. In some embodiments, R^{1a} is C₃₋₁₂ cycloalkyl optionally substituted with R⁶, such as cyclopropyl, cyclobutyl, cyclopentyl, or cyclohexyl, each of which is independently optionally substituted with R⁶. In some embodiments, R^{1a} is C₃₋₁₂ cycloalkyl which is unsubstituted, such as cyclopropyl, cyclobutyl, cyclopentyl, or cyclohexyl. In some embodiments, R^{1a} is C₆₋₁₄ aryl optionally substituted with R⁶, such as phenyl or naphthyl, each of which is independently optionally substituted with R⁶. In some embodiments, R^{1a} is C₆₋₁₄ aryl, which is unsubstituted, such as phenyl or naphthyl. In some embodiments, R^{1a} is phenyl optionally substituted with R⁶. In some embodiments, R^{1a} is phenyl. In some embodiments, R^{1a} is 5- to 10-membered heteroaryl optionally substituted with R⁶. In some embodiments, R^{1a} is 5- or 6-membered heteroaryl optionally substituted with R⁶, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl, each of which is independently optionally substituted with R⁶. In some embodiments, R^{1a} is 5- or 6-membered heteroaryl which is unsubstituted, such as pyridinyl, pyrazinyl, pyridazinyl, primidinyl, triazinyl, pyrrolyl, pyrazolyl, imidazolyl, triazolyl, tetrazolyl, oxazolyl, thiazolyl, thiazolyl, or furanyl. In some embodiments, R^{1a} is 3- to 12-membered heterocyclyl optionally substituted with R⁶. In some embodiments, R^{1a} is 5- or 6-membered heterocyclyl optionally substituted with R⁶, such as tetrahydrofuranyl, pyrrolidinyl, piperidinyl, piperazinyl, morpholinyl, or thiomorpholinyl, each of which is independently optionally substituted with R⁶. In some embodiments, R^{1a} is 5- or 6-membered heterocyclyl which is unsubstituted, such as tetrahydrofuranyl, pyrrolidinyl, piperidinyl, piperazinyl, morpholinyl, or thiomorpholinyl. In some embodiments, R^{1a} is C₁₋₆ alkyl, C₃₋₁₂ cycloalkyl, or 3- to 12-membered heterocyclyl, each of which is independently optionally substituted with R⁶.

In some embodiments of a compound of formula (I), or any related formula, or a stereoisomer, tautomer, prodrug, or a pharmaceutically acceptable salt of any of the foregoing, R⁶ is —OR^{6a}, C₃₋₆ cycloalkyl, 3- to 12-membered heterocyclyl, or C₆₋₁₄ aryl, wherein the C₃₋₆ cycloalkyl, 3- to 12-membered heterocyclyl, and C₆₋₁₄ aryl of R⁶ are each independently optionally substituted with halogen or hydroxyl, and wherein R^{6a} is H or C₁₋₆ alkyl. In some embodiments, R⁶ is —OR^{6a}, wherein R^{6a} is H or C₁₋₆ alkyl. In some embodiments, R⁶ —OH or methoxy. In some embodiments, R⁶ is C₃₋₆ cycloalkyl optionally substituted with halogen, such as cyclopropyl, cyclobutyl, cyclopentyl, or cyclohexyl, each of which is independently optionally substituted with halogen. In some embodiments, R⁶ is C₃₋₆ cycloalkyl, such as cyclopropyl, cyclobutyl, cyclopentyl, or cyclohexyl. In some embodiments, R⁶ is 3- to 12-membered heterocyclyl optionally substituted with halogen. In some embodiments, R⁶ is 5- or 6-membered heterocyclyl optionally substituted with halogen, such as tetrahydrofuranyl, pyrrolidinyl, piperidinyl, piperazinyl, morpholinyl, or thiomorpholinyl, each of which is independently optionally substituted with halogen. In some embodiments, R⁶ is 5- or 6-membered heterocyclyl which is unsubstituted, such as tetrahydrofuranyl, pyrrolidinyl, piperidinyl, piperazinyl, morpholinyl, or thiomorpholinyl. In some embodiments, R⁶ is C₆₋₁₄ aryl, which is unsubstituted, such as phenyl or naphthyl. In some embodiments, R⁶ is phenyl optionally substituted with halogen. In some embodiments, R⁶ is phenyl.

In some embodiments of a compound of formula (I), or any related formula, or a stereoisomer, tautomer, prodrug, or a pharmaceutically acceptable salt of any of the foregoing, R^{1a} is

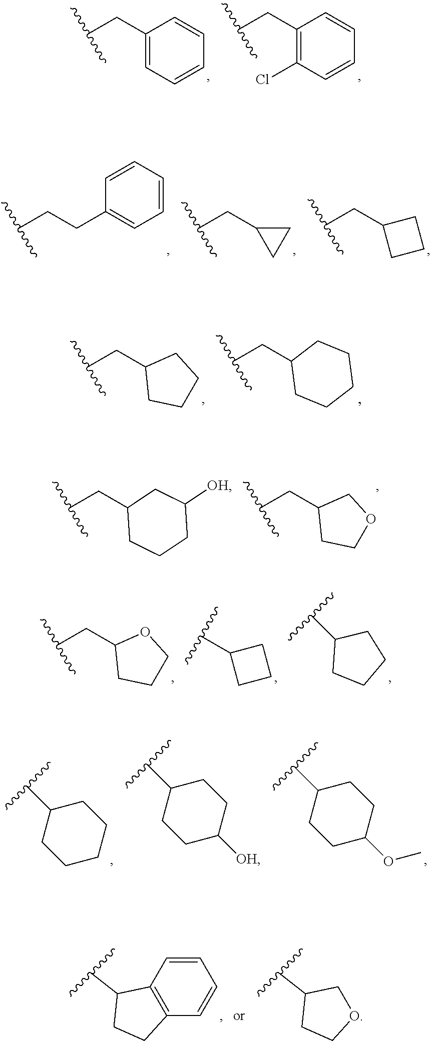

In some embodiments, R^{1a} is

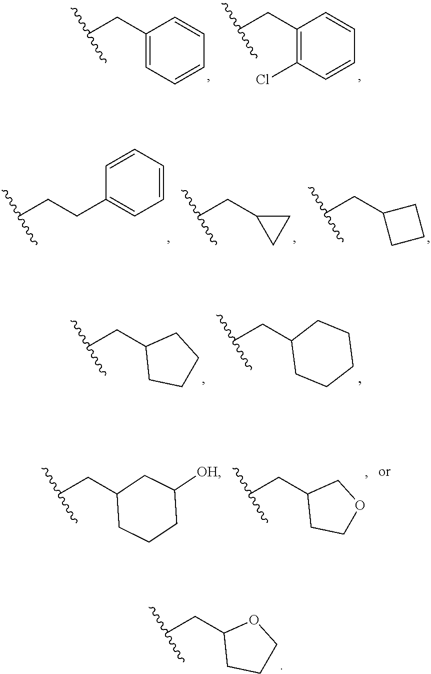

In some embodiments, R^{1a} is

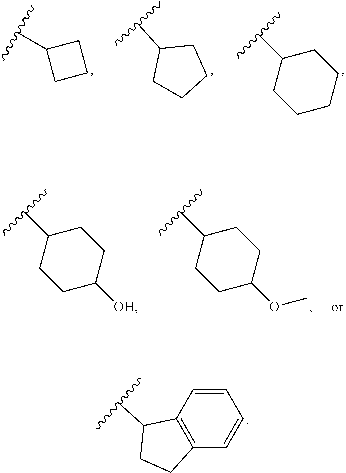

In some embodiments, R^{1a} is

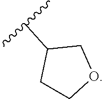 [SMILES: *C1CCOC1]

In some embodiments, R^{1a} is

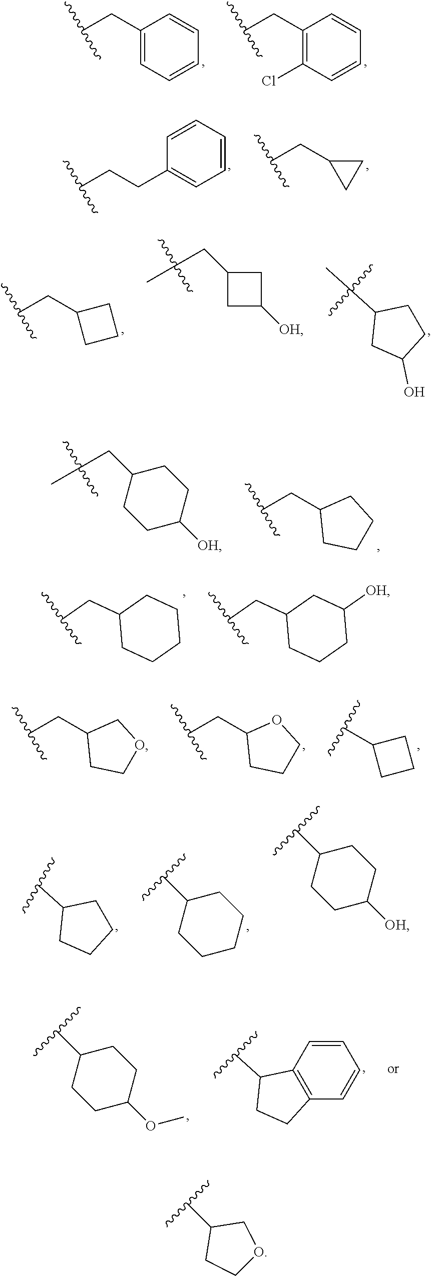

In some embodiments of a compound of formula (I), or any related formula, or a stereoisomer, tautomer, prodrug, or a pharmaceutically acceptable salt of any of the foregoing, R^{1b} is H. In some embodiments, R^{1b} is C₁₋₆ alkyl optionally substituted with R⁶, such as methyl, ethyl, n-propyl, isopropyl, n-butyl, t-butyl, isobutyl, or sec-butyl, each of which is independently optionally substituted with R⁶. In some embodiments, R^{1b} is C₁₋₆ alkyl such as methyl, ethyl, n-propyl, isopropyl, n-butyl, t-butyl, isobutyl, or sec-butyl. In some embodiments, R^{1b} is C₃₋₁₂ cycloalkyl optionally substituted with R⁶, such as cyclopropyl, cyclobutyl, cyclopentyl, or cyclohexyl, each of which is independently optionally substituted with R⁶. In some embodiments, R^{1b} is C₃₋₁₂ cycloalkyl which is unsubstituted, such as cyclopropyl, cyclobutyl, cyclopentyl, or cyclohexyl. In some embodiments, R^{1b} is C₆₋₁₄ aryl optionally substituted with R⁶, such as phenyl or naphthyl, each of which is independently optionally substituted with R⁶. In some embodiments, R^{1b} is C₆₋₁₄ aryl, which is unsubstituted, such as phenyl or naphthyl. In some embodiments, R^{1b} is phenyl optionally substituted with R⁶. In some embodiments, R^{1b} is phenyl. In some embodiments, R^{1b} is 5- to 10-membered heteroaryl optionally substituted with R⁶. In some embodiments, R^{1b} is 5- or 6-membered heteroaryl optionally substituted with R⁶, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl, each of which is independently optionally substituted with R⁶.

In some embodiments, R^{1b} is 5- or 6-membered heteroaryl which is unsubstituted, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R^{1b} is 3- to 12-membered heterocyclyl optionally substituted with R⁶. In some embodiments, R^{1b} is 5- or 6-membered heterocyclyl optionally substituted with R⁶, such as tetrahydrofuranyl, pyrrolidinyl, piperidinyl, piperazinyl, morpholinyl, or thiomorpholinyl, each of which is independently optionally substituted with R⁶. In some embodiments, R^{1b} is 5- or 6-membered heterocyclyl which is unsubstituted, such as tetrahydrofuranyl, pyrrolidinyl, piperidinyl, piperazinyl, morpholinyl, or thiomorpholinyl. In some embodiments, R^{1b} is H or C₁₋₆ alkyl. In some embodiments, R^{1b} is H or methyl.

In some embodiments of a compound of formula (I), or any related formula, or a stereoisomer, tautomer, prodrug, or a pharmaceutically acceptable salt of any of the foregoing, R^{1a} and R^{1b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN. In some embodiments, R^{1a} and R^{1b} are taken together with the nitrogen atom to which they attach to form a 3- to 12-membered heterocyclyl which is unsubstituted. In some embodiments, R^{1a} and R^{1b} are taken together with the nitrogen atom to which they attach to form a 3- to 12-membered heterocyclyl, which is optionally substituted with R⁶. In some embodiments, R^{1a} and R^{1b} are taken together with the nitrogen atom to which they attach to form

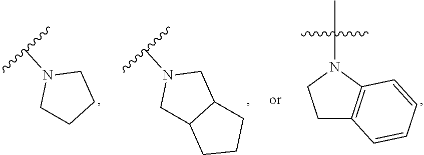 [SMILES: *N1CCCC1 ; *N1CC2CCCC2C1 ; *N1CCc2ccccc21]

In some embodiments, R^{1a} and R^{1b} are taken together with the nitrogen atom to which they attach to form

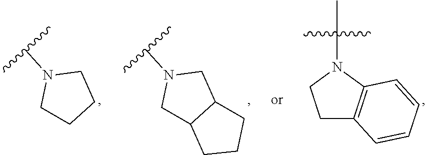 [SMILES: *N1CCCC1 ; *N1CC2CCCC2C1 ; *N1CCc2ccccc21]

each is optionally substituted with R⁶. In some embodiments, R^{1a} and R^{1b} are taken together with the nitrogen atom to which they attach to form

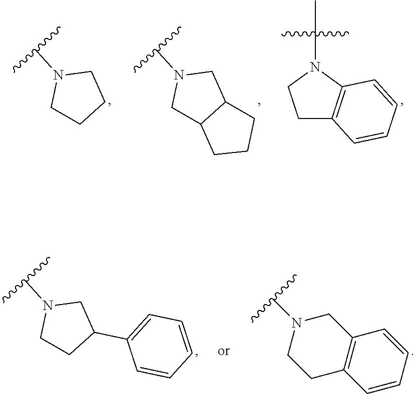

In some embodiments of a compound of formula (I), or any related formula, or a stereoisomer, tautomer, prodrug, or a pharmaceutically acceptable salt of any of the foregoing, R² is H, C₁₋₆ alkyl, C₂₋₆ alkenyl, C₂₋₆ alkynyl, halogen, —CN, —OR^{2a}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently optionally substituted with R⁷. In some embodiments, R² is C₁₋₆ alkyl, C₂₋₆ alkenyl, C₂₋₆ alkynyl, C₃₋₁₂ cycloalkyl, C₆₋₁₄ aryl, 5- to 10-membered heteroaryl, or 3- to 12-membered heterocyclyl, each of which is independently optionally substituted with R⁷. In some embodiments, R² is C₁₋₆ alkyl optionally substituted with R⁷, such as methyl, ethyl, n-propyl, isopropyl, n-butyl, t-butyl, isobutyl, or sec-butyl, each of which is independently optionally substituted with R⁷. In some embodiments, R² is C₁₋₆ alkyl such as methyl, ethyl, n-propyl, isopropyl, n-butyl, t-butyl, isobutyl, or sec-butyl. In some embodiments, R² is C₂₋₆ alkenyl optionally substituted with R⁷, such as ethenyl, prop-1-enyl, prop-2-enyl, 2-methylprop-1-enyl, but-1-enyl, but-2-enyl, or but-3-enyl, each of which is independently optionally substituted with R⁷. In some embodiments, R² is C₂₋₆ alkenyl, such as ethenyl, prop-1-enyl, prop-2-enyl, 2-methylprop-1-enyl, but-1-enyl, but-2-enyl, or but-3-enyl. In some embodiments, R² is C₂₋₆ alkynyl optionally substituted with R⁷, such as ethynyl, prop-1-ynyl, prop-2-ynyl, but-1-ynyl, but-2-ynyl, or but-3-ynyl, each of which is independently optionally substituted with R⁷. In some embodiments, R² is C₂₋₆ alkynyl, such as ethynyl, prop-1-ynyl, prop-2-ynyl, but-1-ynyl, but-2-ynyl, or but-3-ynyl. In some embodiments, R² is halogen, such as fluoro, chloro, or bromo. In some embodiments, R² is chloro. In some embodiments, R² is C₃₋₁₂ cycloalkyl optionally substituted with R⁷. In some embodiments, R² is C₃₋₆ cycloalkyl optionally substituted with R⁷, such as cyclopropyl, cyclobutyl, cyclopentyl, or cyclohexyl, each of which is independently optionally substituted with R⁷. In some embodiments, R² is C₃₋₆ cycloalkyl which is unsubstituted, such as cyclopropyl, cyclobutyl, cyclopentyl, or cyclohexyl. In some embodiments, R² is C₆₋₁₄ aryl optionally substituted with R⁷, such as phenyl or naphthyl, each of which is independently optionally substituted with R⁷. In some embodiments, R² is C₆₋₁₄ aryl, which is unsubstituted, such as phenyl or naphthyl. In some embodiments, R² is phenyl optionally substituted with R⁷. In some embodiments, R² is phenyl. In some embodiments, R² is 5- to 10-membered heteroaryl optionally substituted with R⁷. In some embodiments, R² is 5- or 6-membered heteroaryl optionally substituted with R⁷, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl, each of which is independently optionally substituted with R⁷. In some embodiments, R² is 5- or 6-membered heteroaryl which is unsubstituted, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R² is 3- to 12-membered heterocyclyl optionally substituted with R⁷. In some embodiments, R² is 5- or 6-membered heterocyclyl optionally substituted with R⁷, such as tetrahydrofuranyl, pyrrolidinyl, piperidinyl, piperazinyl, morpholinyl, or thiomorpholinyl, each of which is independently optionally substituted with R⁷. In some embodiments, R² is 5- or 6-membered heterocyclyl which is unsubstituted, such as tetrahydrofuranyl, pyrrolidinyl, piperidinyl, piperazinyl, morpholinyl, or thiomorpholinyl. In some embodiments, R² is H, halogen, C₁₋₆ alkyl, or C₂₋₆ alkenyl, wherein the C₁₋₆ alkyl and C₂₋₆ alkenyl are each independently optionally substituted with R⁷. In some embodiments, R² is H, halogen, C₁₋₆ alkyl, or C₂₋₆ alkenyl. In some embodiments, R² is H, chloro, —CH₃, —CH₂CH₃, or —CH═CH₂. In some embodiments, R² is H, halogen, C₁₋₆ alkyl, C₃₋₆ cycloalkyl, or C₂₋₆ alkenyl, wherein the C₁₋₆ alkyl, C₃₋₆ cycloalkyl, and C₂₋₆ alkenyl are each independently optionally substituted with R⁷. In some embodiments, R² is H, halogen, C₁₋₆ alkyl, C₃₋₆ cycloalkyl, or C₂₋₆ alkenyl. In some embodiments, R² is H, chloro, —CH₃, —CH₂CH₃, cyclopropyl, or —CH═CH₂.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R¹ of formula (I) may be combined with every description, variation, embodiment or aspect of Y, Z, X¹, X², X³, and R¹-R⁵ the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, in some embodiments of a compound of formula (I) or any related formula where applicable, or a stereoisomer, tautomer, prodrug, or a pharmaceutically acceptable salt of any of the foregoing, Y is —CR^{Y}—, wherein R^{Y} is H; Z is —CR^{Z}—, wherein R^{Z} is H or halogen; X¹ is —OH or H; X² is H, halogen, or C₁₋₆ alkyl; X³ is H or —CN; R^{1a} is C₁₋₆ alkyl, C₃₋₁₂ cycloalkyl, or 3- to 12-membered heterocyclyl, each of which is independently optionally substituted with R⁶, wherein R⁶ is —OR^{6a}, C₃₋₆ cycloalkyl, 3- to 12-membered heterocyclyl, or C₆₋₁₄ aryl, wherein the C₃₋₆ cycloalkyl, 3- to 12-membered heterocyclyl, and C₆₋₁₄ aryl of R⁶ are each independently optionally substituted with halogen or hydroxyl, and wherein R^{6a} is H or C₁₋₆ alkyl; R^{1b} is H or C₁₋₆ alkyl, or R^{1a} and R^{1b} are taken together with the nitrogen atom to which they attach to form a 3- to 12-membered heterocyclyl; R² is H, halogen, C₁₋₆ alkyl, or C₂₋₆ alkenyl, wherein the C₁₋₆ alkyl and C₂₋₆ alkenyl are each independently optionally substituted with R⁷; R³ is H; R⁴ is H; and R⁵ is H.

In some embodiments, provided is compound selected from the compounds in Table 1, or a stereoisomer, tautomer, solvate, prodrug or salt thereof. In some embodiments, provided is compound selected from the compounds in Table 1, or a stereoisomer, tautomer, prodrug, or a pharmaceutically acceptable salt of any of the foregoing. In some embodiments, provided is a compound selected from the compounds in Table 1, or a pharmaceutically acceptable salt thereof. In some embodiments, provided is a compound selected from the compounds in Table 1. Although certain compounds described in Table 1 are presented as specific stereoisomers and/or in a non-stereochemical form, it is understood that any or all stereochemical forms, including any enantiomeric or diastereomeric forms, and any tautomers or other forms of any of the compounds of Table 1 are herein described.

TABLE 1

| No. | Structure |
|---|---|
| 1 | 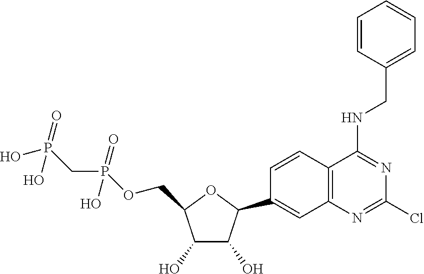 [SMILES: O=P(O)(O)CP(=O)(O)OC[C@H]1O[C@@H](c2ccc3c(NCc4ccccc4)nc(Cl)nc3c2)[C@H](O)[C@@H]1O] |
| 2 | 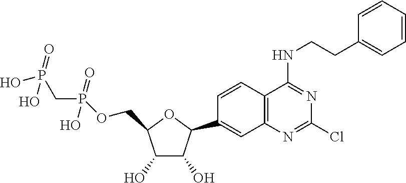 [SMILES: O=P(O)(O)CP(=O)(O)OC[C@H]1O[C@@H](c2ccc3c(NCCc4ccccc4)nc(Cl)nc3c2)[C@H](O)[C@@H]1O] |
| 3 | 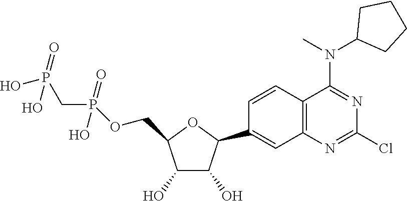 [SMILES: CN(c1nc(Cl)nc2cc([C@@H]3O[C@H](COP(=O)(O)CP(=O)(O)O)[C@@H](O)[C@H]3O)ccc12)C1CCCC1] |
| 4 | 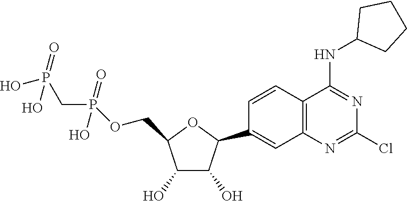 [SMILES: O=P(O)(O)CP(=O)(O)OC[C@H]1O[C@@H](c2ccc3c(NC4CCCC4)nc(Cl)nc3c2)[C@H](O)[C@@H]1O] |
| 5 | 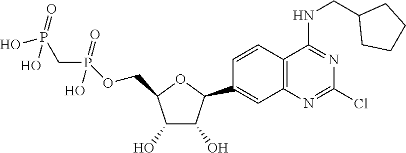 [SMILES: O=P(O)(O)CP(=O)(O)OC[C@H]1O[C@@H](c2ccc3c(NCC4CCCC4)nc(Cl)nc3c2)[C@H](O)[C@@H]1O] |
| 6 | 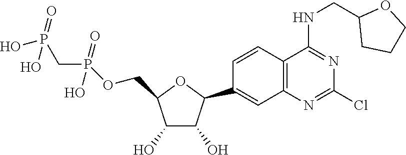 [SMILES: O=P(O)(O)CP(=O)(O)OC[C@H]1O[C@@H](c2ccc3c(NCC4CCCO4)nc(Cl)nc3c2)[C@H](O)[C@@H]1O] |
| 7 | 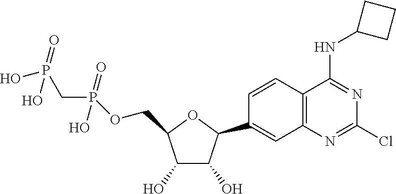 [SMILES: O=P(O)(O)CP(=O)(O)OC[C@H]1O[C@@H](c2ccc3c(NC4CCC4)nc(Cl)nc3c2)[C@H](O)[C@@H]1O] |
| 8 | 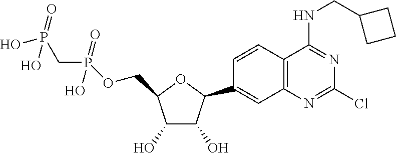 [SMILES: O=P(O)(O)CP(=O)(O)OC[C@H]1O[C@@H](c2ccc3c(NCC4CCC4)nc(Cl)nc3c2)[C@H](O)[C@@H]1O] |
| 9 | 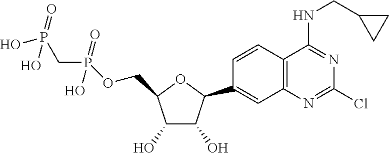 [SMILES: O=P(O)(O)CP(=O)(O)OC[C@H]1O[C@@H](c2ccc3c(NCC4CC4)nc(Cl)nc3c2)[C@H](O)[C@@H]1O] |
| 10 | 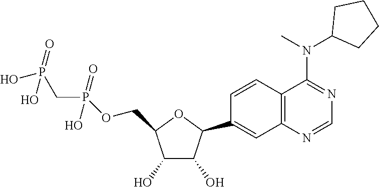 [SMILES: CN(c1ncnc2cc([C@@H]3O[C@H](COP(=O)(O)CP(=O)(O)O)[C@@H](O)[C@H]3O)ccc12)C1CCCC1] |
| 11 | 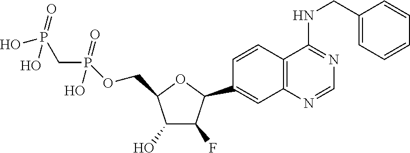 [SMILES: O=P(O)(O)CP(=O)(O)OC[C@H]1O[C@@H](c2ccc3c(NCc4ccccc4)ncnc3c2)[C@@H](F)[C@@H]1O] |
| 12 | 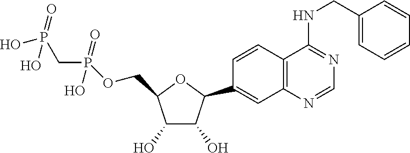 [SMILES: O=P(O)(O)CP(=O)(O)OC[C@H]1O[C@@H](c2ccc3c(NCc4ccccc4)ncnc3c2)[C@H](O)[C@@H]1O] |
| 13 | 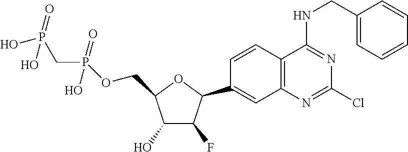 [SMILES: O=P(O)(O)CP(=O)(O)OC[C@H]1O[C@@H](c2ccc3c(NCc4ccccc4)nc(Cl)nc3c2)[C@@H](F)[C@@H]1O] |
| 14 | 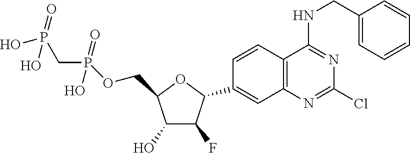 [SMILES: O=P(O)(O)CP(=O)(O)OC[C@H]1O[C@H](c2ccc3c(NCc4ccccc4)nc(Cl)nc3c2)[C@@H](F)[C@@H]1O] |
| 15 | 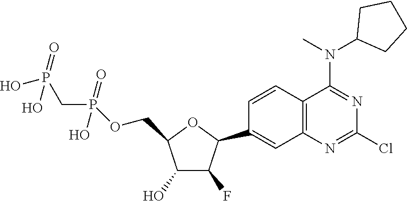 [SMILES: CN(c1nc(Cl)nc2cc([C@@H]3O[C@H](COP(=O)(O)CP(=O)(O)O)[C@@H](O)[C@@H]3F)ccc12)C1CCCC1] |
| 16 | 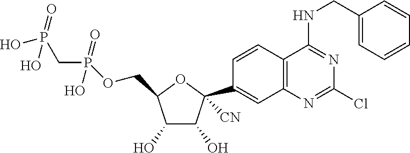 [SMILES: N#C[C@@]1(c2ccc3c(NCc4ccccc4)nc(Cl)nc3c2)O[C@H](COP(=O)(O)CP(=O)(O)O)[C@@H](O)[C@H]1O] |
| 17 | 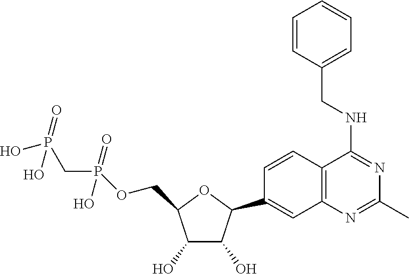 [SMILES: Cc1nc(NCc2ccccc2)c2ccc([C@@H]3O[C@H](COP(=O)(O)CP(=O)(O)O)[C@@H](O)[C@H]3O)cc2n1] |
| 18 | 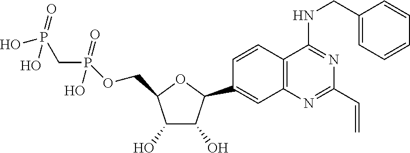 [SMILES: C=Cc1nc(NCc2ccccc2)c2ccc([C@@H]3O[C@H](COP(=O)(O)CP(=O)(O)O)[C@@H](O)[C@H]3O)cc2n1] |
| 19 | 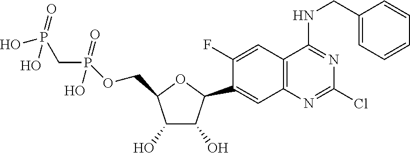 [SMILES: O=P(O)(O)CP(=O)(O)OC[C@H]1O[C@@H](c2cc3nc(Cl)nc(NCc4ccccc4)c3cc2F)[C@H](O)[C@@H]1O] |
| 20 | 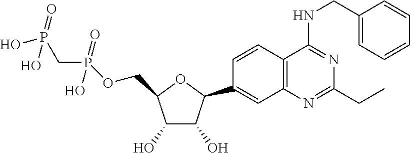 [SMILES: CCc1nc(NCc2ccccc2)c2ccc([C@@H]3O[C@H](COP(=O)(O)CP(=O)(O)O)[C@@H](O)[C@H]3O)cc2n1] |
| 21 | 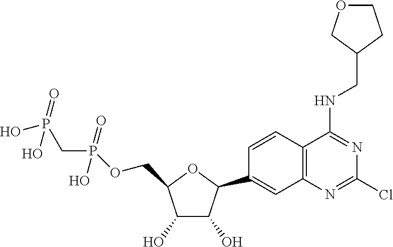 [SMILES: O=P(O)(O)CP(=O)(O)OC[C@H]1O[C@@H](c2ccc3c(NCC4CCOC4)nc(Cl)nc3c2)[C@H](O)[C@@H]1O] |
| 22 | 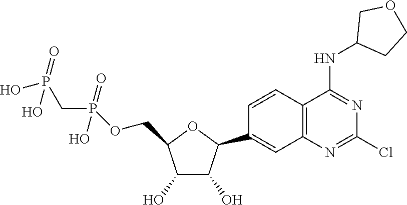 [SMILES: O=P(O)(O)CP(=O)(O)OC[C@H]1O[C@@H](c2ccc3c(NC4CCOC4)nc(Cl)nc3c2)[C@H](O)[C@@H]1O] |
| 23 | 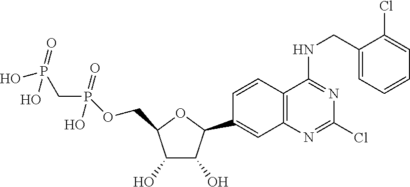 [SMILES: O=P(O)(O)CP(=O)(O)OC[C@H]1O[C@@H](c2ccc3c(NCc4ccccc4Cl)nc(Cl)nc3c2)[C@H](O)[C@@H]1O] |
| 24 | 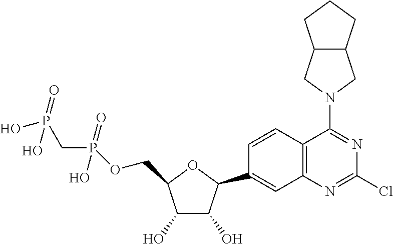 [SMILES: O=P(O)(O)CP(=O)(O)OC[C@H]1O[C@@H](c2ccc3c(N4CC5CCCC5C4)nc(Cl)nc3c2)[C@H](O)[C@@H]1O] |
| 25 | 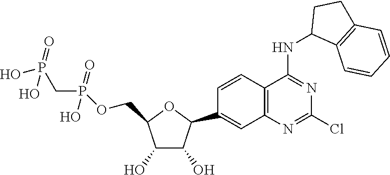 [SMILES: O=P(O)(O)CP(=O)(O)OC[C@H]1O[C@@H](c2ccc3c(NC4CCc5ccccc54)nc(Cl)nc3c2)[C@H](O)[C@@H]1O] |
| 26 | 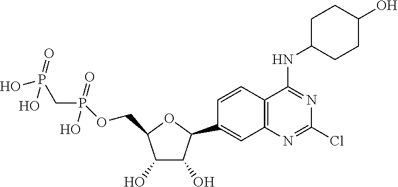 [SMILES: O=P(O)(O)CP(=O)(O)OC[C@H]1O[C@@H](c2ccc3c(NC4CCC(O)CC4)nc(Cl)nc3c2)[C@H](O)[C@@H]1O] |
| 27 | 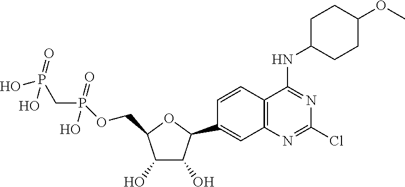 [SMILES: COC1CCC(Nc2nc(Cl)nc3cc([C@@H]4O[C@H](COP(=O)(O)CP(=O)(O)O)[C@@H](O)[C@H]4O)ccc23)CC1] |
| 28 | 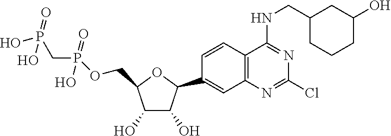 [SMILES: O=P(O)(O)CP(=O)(O)OC[C@H]1O[C@@H](c2ccc3c(NCC4CCCC(O)C4)nc(Cl)nc3c2)[C@H](O)[C@@H]1O] |
| 29 | 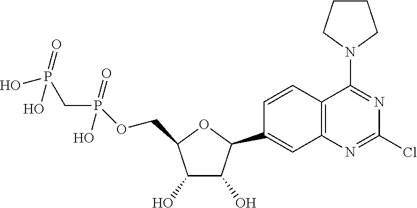 [SMILES: O=P(O)(O)CP(=O)(O)OC[C@H]1O[C@@H](c2ccc3c(N4CCCC4)nc(Cl)nc3c2)[C@H](O)[C@@H]1O] |
| 30 | 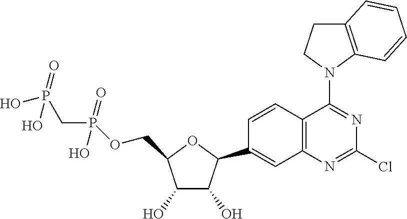 [SMILES: O=P(O)(O)CP(=O)(O)OC[C@H]1O[C@@H](c2ccc3c(N4CCc5ccccc54)nc(Cl)nc3c2)[C@H](O)[C@@H]1O] |
| 31 | 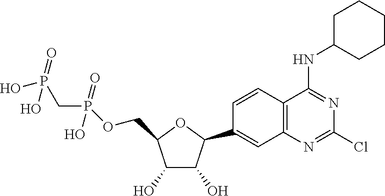 [SMILES: O=P(O)(O)CP(=O)(O)OC[C@H]1O[C@@H](c2ccc3c(NC4CCCCC4)nc(Cl)nc3c2)[C@H](O)[C@@H]1O] |
| 32 | 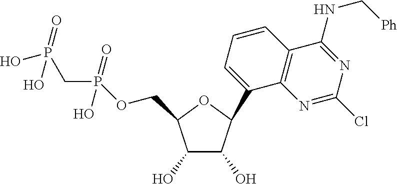 [SMILES: O=P(O)(O)CP(=O)(O)OC[C@H]1O[C@@H](c2cccc3c(NCc4ccccc4)nc(Cl)nc23)[C@H](O)[C@@H]1O] |
| 33 | 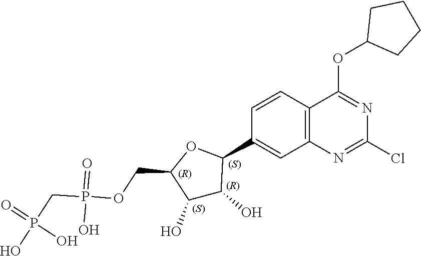 |
| 34 | 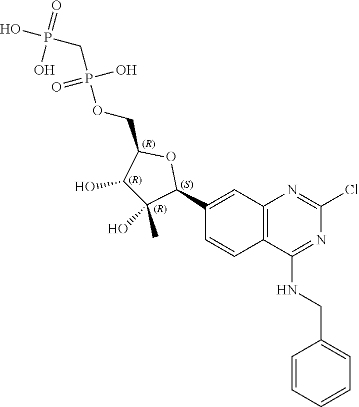 |
| 35 | 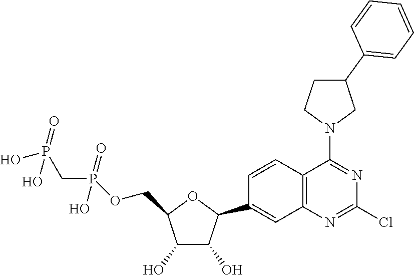 [SMILES: O=P(O)(O)CP(=O)(O)OC[C@H]1O[C@@H](c2ccc3c(N4CCC(c5ccccc5)C4)nc(Cl)nc3c2)[C@H](O)[C@@H]1O] |
| 36 | 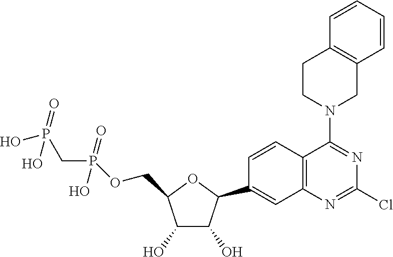 [SMILES: O=P(O)(O)CP(=O)(O)OC[C@H]1O[C@@H](c2ccc3c(N4CCc5ccccc5C4)nc(Cl)nc3c2)[C@H](O)[C@@H]1O] |
| 37 | 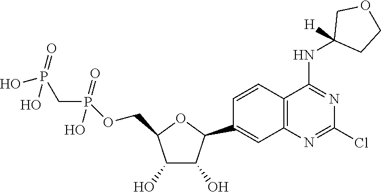 [SMILES: O=P(O)(O)CP(=O)(O)OC[C@H]1O[C@@H](c2ccc3c(N[C@H]4CCOC4)nc(Cl)nc3c2)[C@H](O)[C@@H]1O] |
| 38 | 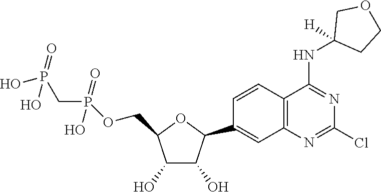 [SMILES: O=P(O)(O)CP(=O)(O)OC[C@H]1O[C@@H](c2ccc3c(N[C@@H]4CCOC4)nc(Cl)nc3c2)[C@H](O)[C@@H]1O] |
| 39 | 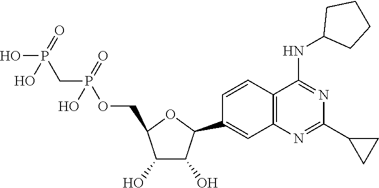 [SMILES: O=P(O)(O)CP(=O)(O)OC[C@H]1O[C@@H](c2ccc3c(NC4CCCC4)nc(C4CC4)nc3c2)[C@H](O)[C@@H]1O] |
| 40 | 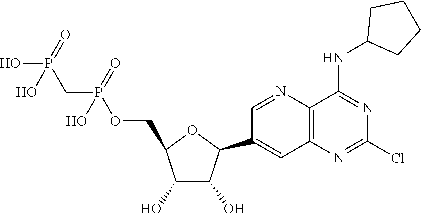 [SMILES: O=P(O)(O)CP(=O)(O)OC[C@H]1O[C@@H](c2cnc3c(NC4CCCC4)nc(Cl)nc3c2)[C@H](O)[C@@H]1O] |
| 41 | 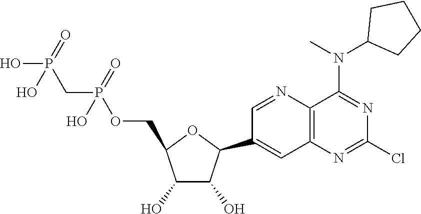 [SMILES: CN(c1nc(Cl)nc2cc([C@@H]3O[C@H](COP(=O)(O)CP(=O)(O)O)[C@@H](O)[C@H]3O)cnc12)C1CCCC1] |
| 42 | 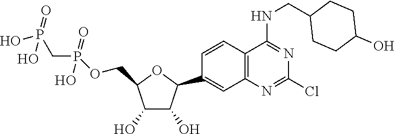 [SMILES: O=P(O)(O)CP(=O)(O)OC[C@H]1O[C@@H](c2ccc3c(NCC4CCC(O)CC4)nc(Cl)nc3c2)[C@H](O)[C@@H]1O] |
| 43 | 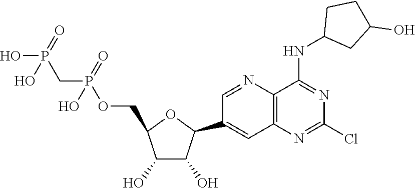 [SMILES: O=P(O)(O)CP(=O)(O)OC[C@H]1O[C@@H](c2cnc3c(NC4CCC(O)C4)nc(Cl)nc3c2)[C@H](O)[C@@H]1O] |
| 44 | 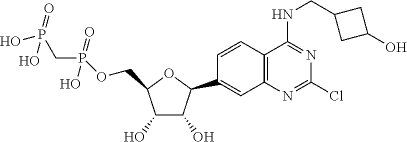 [SMILES: O=P(O)(O)CP(=O)(O)OC[C@H]1O[C@@H](c2ccc3c(NCC4CC(O)C4)nc(Cl)nc3c2)[C@H](O)[C@@H]1O] |
| 45 | 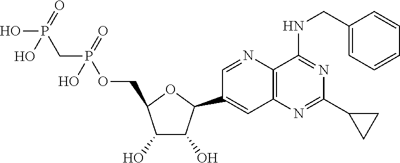 [SMILES: O=P(O)(O)CP(=O)(O)OC[C@H]1O[C@@H](c2cnc3c(NCc4ccccc4)nc(C4CC4)nc3c2)[C@H](O)[C@@H]1O] |
| 46 | 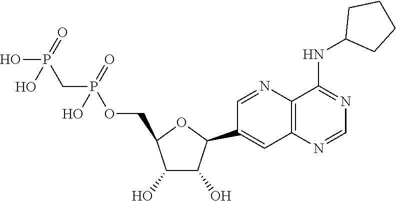 [SMILES: O=P(O)(O)CP(=O)(O)OC[C@H]1O[C@@H](c2cnc3c(NC4CCCC4)ncnc3c2)[C@H](O)[C@@H]1O] |

Also provided are salts of compounds referred to herein, such as pharmaceutically acceptable salts. The present disclosure also includes any or all of the stereochemical forms, including any enantiomeric or diastereomeric forms, and any tautomers or other forms of the compounds described. Thus, if a particular stereochemical form, such as a specific enantiomeric form or diastereomeric form, is depicted for a given compound, then it is understood that any or all stereochemical forms, including any enantiomeric or diastereomeric forms, and any tautomers or other forms of any of that same compound are herein described. Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The disclosure also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds described herein include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances. Isotopically-labeled compounds described herein can generally be prepared by standard methods and techniques known to those skilled in the art or by procedures similar to those described in the accompanying Examples substituting appropriate isotopically-labeled reagents in place of the corresponding non-labeled reagent.

The disclosure also includes any or all metabolites of any of the compounds described. The metabolites may include any chemical species generated by a biotransformation of any of the compounds described, such as intermediates and products of metabolism of the compound, such as would be generated in vivo following administration to a human.

Solvates and/or polymorphs of a compound provided herein, or a salt thereof are also contemplated. Solvates contain either stoichiometric or non-stoichiometric amounts of a solvent and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### Pharmaceutical Compositions and Formulations

Pharmaceutical compositions of any of the compounds detailed herein are embraced by this disclosure. Thus, the present disclosure includes pharmaceutical compositions comprising a compound as detailed herein, or a stereoisomer, tautomer, prodrug, or a pharmaceutically acceptable salt of any of the foregoing, and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein, or a stereoisomer, tautomer, prodrug, or a pharmaceutically acceptable salt of any of the foregoing, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

A compound detailed herein, or a stereoisomer, tautomer, prodrug, or a pharmaceutically acceptable salt of any of the foregoing, can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

A compound detailed herein, or a stereoisomer, tautomer, prodrug, or a pharmaceutically acceptable salt of any of the foregoing, may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided. In some embodiments, the composition is for use as a human or veterinary medicament. In some embodiments, the composition is for use in a method described herein. In some embodiments, the composition is for use in the treatment of a disease or disorder described herein.

### Methods of Use

Compounds and compositions detailed herein, such as a pharmaceutical composition containing a compound of any formula provided herein, or a stereoisomer, tautomer, prodrug, or a pharmaceutically acceptable salt of any of the foregoing, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease or disorder in an individual in need thereof comprising administering a compound describes herein or any embodiment, variation, or aspect thereof, or a pharmaceutically acceptable salt thereof. In some embodiments, the compound, pharmaceutically acceptable salt thereof, or composition is administered to the individual according to a dosage and/or method of administration described herein.

Compounds and compositions detailed herein can inhibit the activity of the CD73. For example, the compounds of the disclosure can be used to inhibit activity of CD73 in a cell or in an individual or patient in need of inhibition of the enzyme by administering an inhibiting amount of a compound of the disclosure to the cell, individual, or patient.

Compounds and compositions detailed herein are useful in the treatment of cancer. Examples of cancers include, without limitation, bladder cancer, leukemia, glioma, glioblastoma, melanoma, ovarian cancer, thyroid cancer, esophageal cancer, prostate cancer, lung cancer, colorectal cancer, pancreatic cancer, skin cancer, liver cancer, gastric cancer, head & neck cancer, and breast cancer.

Compounds and compositions detailed herein are useful in the treatment of immune-related disease. The term “immune-related disease” means a disease in which a component of the immune system causes, mediates or otherwise contributes to a morbidity. Also included are diseases in which stimulation or intervention of the immune response has an ameliorative effect on progression of the disease. Examples of immune-related diseases include, without limitation, immune-mediated inflammatory diseases, non-immune-mediated inflammatory diseases, infectious diseases, immunodeficiency diseases, and neoplasia, etc.

### Combinations

In certain aspects, compounds or compositions described herein are administered to an individual for treatment of a disease in combination with one or more additional pharmaceutical agents that can treat the disease. For example, in some embodiments, an effective amount of the compound of formula (I) or any related formula, or a stereoisomer, tautomer, prodrug, or a pharmaceutically acceptable salt of any of the foregoing, is administered to an individual for the treatment of a disease such as cancer in combination with one or more additional therapeutic agents. In some embodiments, the additional therapeutic agent comprises a checkpoint inhibitor. In some embodiments, the checkpoint inhibitor comprises a cytotoxic T lymphocyte associated protein 4 (CTLA-4) inhibitor, programmed cell death protein 1 (PD-1) inhibitor, or programmed death ligand 1 (PD-L1) inhibitor. In some embodiments, the checkpoint inhibitor comprises a CTLA-4 inhibitor such as ipilimumab. In some embodiments, the checkpoint inhibitor comprises a PD-1 inhibitor such as nivolumab or pembrolizumab. In some embodiments, the checkpoint inhibitor comprises a PD-L1 inhibitor such as atezolizumab.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### Articles of Manufacture and Kits

The present disclosure further provides articles of manufacture comprising a compound described herein or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein or a salt thereof. The kits may be used for any one or more of the uses described herein, and, accordingly, may contain instructions for the treatment of any disease or described herein, for example for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

Certain representative embodiments are provided below.
Embodiment 1. A compound of formula (I):

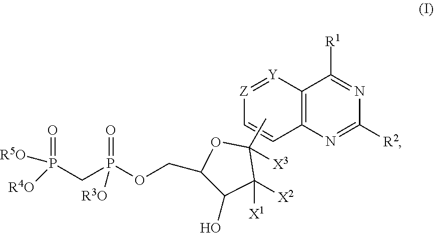

or a stereoisomer, tautomer, prodrug, or a pharmaceutically acceptable salt of any of the foregoing, wherein:
X¹, X², and X³ are each independently H, —CN, C₁₋₆ alkyl, —OR′, or halogen, wherein R′ is H, C₁₋₆ alkyl, C₃₋₁₂ cycloalkyl, 3- to 12-membered heterocyclyl, 5- to 10-membered heteroaryl, or C₆₋₁₄ aryl;
Y is —CR^{Y}— or N, wherein R^{Y} is H, C₁₋₆ alkyl, or halogen;
Z is —CR^{Z}— or N, wherein R^{Z} is H, C₁₋₆ alkyl, or halogen;
R¹ is —NR^{1a}R^{1b} or —OR^{1a}, wherein R^{1a} and R^{1b} are each independently H, C₁₋₆ alkyl, C₃₋₁₂ cycloalkyl, 3- to 12-membered heterocyclyl, 5- to 10-membered heteroaryl, or C₆₋₁₄ aryl, wherein the C₁₋₆ alkyl, C₃₋₁₂ cycloalkyl, 3- to 12-membered heterocyclyl, 5- to 10-membered heteroaryl, and C₆₋₁₄ aryl are each independently optionally substituted with R⁶, or

R^{1a} and R^{1b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
R² is H, C₁₋₆ alkyl, C₂₋₆ alkenyl, C₂₋₆ alkynyl, halogen, —CN, —OR^{2a}, —SR^{2a}, —NR^{2a}R^{2b}, —OC(O)R^{2a}, —NR^{2a}C(O)R^{2b}, —NR^{2a}C(O)OR^{2b}, —NR^{2a}S(O)R^{2b}, —NR^{2a}S(O)₂R^{2b}, C(O)NR^{2a}R^{2b}, —C(O)NR^{2a}S(O)₂R^{2b}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently optionally substituted with R⁷, and wherein:

R^{2a} and R^{2b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{2a} and R^{2b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
R³, R⁴, and R⁵ are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl;
each R⁶ is independently oxo, C₁₋₆ alkyl, C₂₋₆ alkenyl, C₂₋₆ alkynyl, halogen, —CN, —OR^{6a}, —SR^{6a}, —NR^{6a}R^{6b}, —NO₂, —C═NH(OR^{6a}), —C(O)R^{6a}, —OC(O)R^{6a}, —C(O)OR^{6a}, —C(O)NR^{6a}R^{6b}, —OC(O)NR^{6a}R^{6b}, —NR^{6a}C(O)R^{6b}, —NR^{6a}C(O)OR^{6b}, —S(O)R^{6a}, —S(O)₂R^{6a}, —NR^{6a}S(O)R^{6b}, —C(O)NR^{6a}S(O)R^{6b}, —NR^{6a}S(O)₂R^{6b}, —C(O)NR^{6a}S(O)₂R^{6b}, —S(O)NR^{6a}R^{6b}, —S(O)₂NR^{6a}R^{6b}, —P(O)(OR^{6a}) (OR^{6b}), C₃₋₆ cycloalkyl, 3- to 12-membered heterocyclyl, 5- to 10-membered heteroaryl, or C₆₋₁₄ aryl, wherein the C₃₋₆ cycloalkyl, 3- to 12-membered heterocyclyl, 5- to 10-membered heteroaryl, and C₆₋₁₄ aryl are each independently optionally substituted with C₁₋₆ alkyl, C₂₋₆ alkenyl, C₂₋₆ alkynyl, halogen, hydroxyl, C₁₋₆ alkoxy, or —CN, and wherein:

R^{6a} and R^{6b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{6a} and R^{6b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
   each R⁷ is independently oxo, C₁₋₆ alkyl, C₂₋₆ alkenyl, C₂₋₆ alkynyl, halogen, —CN, —SR^{7a}, —NR^{7a}R^{7b}, —NO₂, —C═NH(OR^{7a}), —C(O)R^{7a}, —OC(O)R^{7a}, —C(O)OR^{7a}, —C(O)NR^{7a}R^{7b}, —OC(O)NR^{7a}R^{7b}, —NR^{7a}C(O)R^{7b}, —NR^{7a}C(O)OR^{7b}, —S(O)R^{7a}, —S(O)₂R^{7a}, —NR^{7a}S(O)R^{7b}, —C(O)NR^{7a}S(O)R^{7b}, —NR^{7a}S(O)₂R^{7b}, —C(O)NR^{7a}S(O)₂R^{7b}, —S(O)NR^{7a}R^{7b}, —S(O)₂NR^{7a}R^{7b}, —P(O)(OR^{7a}) (OR^{7b}), C₃₋₆ cycloalkyl, 3- to 12-membered heterocyclyl, 5- to 10-membered heteroaryl, or C₆₋₁₄ aryl, wherein:

R^{7a} and R^{7b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{7a} and R^{7b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN.
Embodiment 2. The compound of embodiment 1, or a stereoisomer, tautomer, prodrug, or a pharmaceutically acceptable salt of any of the foregoing, wherein the compound is of formula (II):

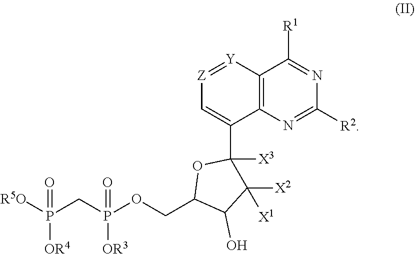

Embodiment 3. The compound of embodiment 1, or a stereoisomer, tautomer, prodrug, or a pharmaceutically acceptable salt of any of the foregoing, wherein the compound is of formula (III):

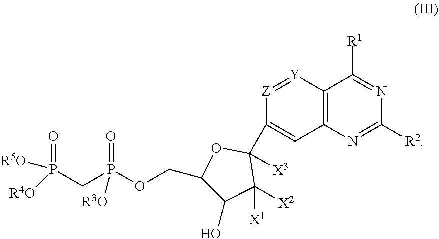

Embodiment 4. The compound of any one of embodiments 1-3, or a stereoisomer, tautomer, prodrug, or a pharmaceutically acceptable salt of any of the foregoing, wherein Y is
Embodiment 5. The compound of any one of embodiments 1-4, or a stereoisomer, tautomer, prodrug, or a pharmaceutically acceptable salt of any of the foregoing, wherein R^{Y} is H.
Embodiment 6. The compound of any one of embodiments 1-5, or a stereoisomer, tautomer, prodrug, or a pharmaceutically acceptable salt of any of the foregoing, wherein Z is —CR^{Z}—.
Embodiment 7. The compound of any one of embodiments 1-6, or a stereoisomer, tautomer, prodrug, or a pharmaceutically acceptable salt of any of the foregoing, wherein R^{Z} is H or halogen.
Embodiment 8. The compound of any one of embodiments 1-7, or a stereoisomer, tautomer, prodrug, or a pharmaceutically acceptable salt of any of the foregoing, wherein X¹ is H or —OH.
Embodiment 9. The compound of any one of embodiments 1-8, or a stereoisomer, tautomer, prodrug, or a pharmaceutically acceptable salt of any of the foregoing, wherein X² is H, halogen, or C₁₋₆ alkyl.
Embodiment 10. The compound of any one of embodiments 1-9, or a stereoisomer, tautomer, prodrug, or a pharmaceutically acceptable salt of any of the foregoing, wherein X³ is H or —CN.
Embodiment 11. The compound of any one of embodiments 1-10, or a stereoisomer, tautomer, prodrug, or a pharmaceutically acceptable salt of any of the foregoing, wherein R¹ is —NR^{1a}R^{1b}.
Embodiment 12. The compound of any one of embodiments 1-10, or a stereoisomer, tautomer, prodrug, or a pharmaceutically acceptable salt of any of the foregoing, wherein R¹ is —OR^{1a}.
Embodiment 13. The compound of any one of embodiments 1-12, or a stereoisomer, tautomer, prodrug, or a pharmaceutically acceptable salt of any of the foregoing, wherein R^{1a} is C₁₋₆ alkyl, C₃₋₁₂ cycloalkyl, or 3- to 12-membered heterocyclyl, each of which is independently optionally substituted with R⁶.
Embodiment 14. The compound of any one of embodiments 1-13, or a stereoisomer, tautomer, prodrug, or a pharmaceutically acceptable salt of any of the foregoing, wherein R⁶ is —OR^{6a}, C₃₋₆ cycloalkyl, 3- to 12-membered heterocyclyl, or C₆₋₁₄ aryl, wherein the C₃₋₆ cycloalkyl, 3- to 12-membered heterocyclyl, and C₆₋₁₄ aryl of R⁶ are each independently optionally substituted with halogen or hydroxyl, and wherein R^{6a} is H or C₁₋₆ alkyl.
Embodiment 15. The compound of any one of embodiments 1-14, or a stereoisomer, tautomer, prodrug, or a pharmaceutically acceptable salt of any of the foregoing, wherein R^{1a} is

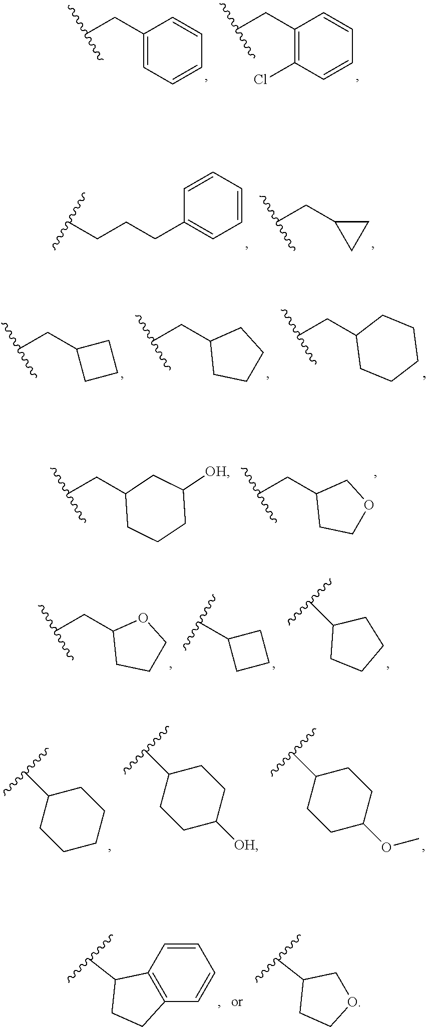

Embodiment 16. The compound of any one of embodiments 1-11 and 13-15, or a stereoisomer, tautomer, prodrug, or a pharmaceutically acceptable salt of any of the foregoing, wherein R^{1b} is H or C₁₋₆ alkyl.
Embodiment 17. The compound of any one of embodiments 1-11, or a stereoisomer, tautomer, prodrug, or a pharmaceutically acceptable salt of any of the foregoing, wherein R^{1a} and R^{1b} are taken together with the nitrogen atom to which they attach to form a 3- to 12-membered heterocyclyl.
Embodiment 18. The compound of embodiment 17, or a stereoisomer, tautomer, prodrug, or a pharmaceutically acceptable salt of any of the foregoing, wherein R^{1a} and R^{1b} are taken together with the nitrogen atom to which they attach to form

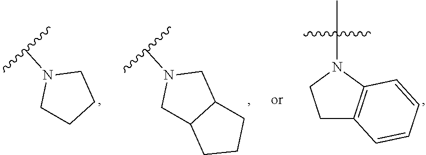 [SMILES: *N1CCCC1 ; *N1CC2CCCC2C1 ; *N1CCc2ccccc21]

Embodiment 19. The compound of any one of embodiments 1-18, or a stereoisomer, tautomer, prodrug, or a pharmaceutically acceptable salt of any of the foregoing, wherein R² is H, halogen, C₁₋₆ alkyl, or C₂₋₆ alkenyl, wherein the C₁₋₆ alkyl and C₂₋₆ alkenyl are each independently optionally substituted with R⁷.
Embodiment 20. The compound of any one of embodiments 1-19, or a stereoisomer, tautomer, prodrug, or a pharmaceutically acceptable salt of any of the foregoing, wherein R² is H, chloro, —CH₃, —CH₂CH₃, or —CH═CH₂.
Embodiment 21. The compound of any one of embodiments 1-20, or a stereoisomer, tautomer, prodrug, or a pharmaceutically acceptable salt of any of the foregoing, wherein R³ is H.
Embodiment 22. The compound of any one of embodiments 1-21, or a stereoisomer, tautomer, prodrug, or a pharmaceutically acceptable salt of any of the foregoing, wherein R⁴ is H.
Embodiment 23. The compound of any one of embodiments 1-22, or a stereoisomer, tautomer, prodrug, or a pharmaceutically acceptable salt of any of the foregoing, wherein R⁵ is H.
Embodiment 24. A compound selected from the group consisting of the compounds in Table 1, or a stereoisomer, tautomer, prodrug, or a pharmaceutically acceptable salt of any of the foregoing.
Embodiment 25. A pharmaceutical composition comprising at least one compound according to any one of embodiments 1-24, or a stereoisomer, tautomer, prodrug, or a pharmaceutically acceptable salt of any of the foregoing, optionally further comprising a pharmaceutically acceptable excipient.
Embodiment 26. A kit comprising at least one compound according to any one of embodiments 1-24, or a stereoisomer, tautomer, prodrug, or a pharmaceutically acceptable salt of any of the foregoing,
Embodiment 27. A method of treating a disease mediated by CD73 in an individual in need thereof, comprising administering to the individual a therapeutically effective amount of a compound according to any one of embodiments 1-24, or a stereoisomer, tautomer, prodrug, or a pharmaceutically acceptable salt of any of the foregoing.
Embodiment 28. The method of embodiment 27, wherein the disease is cancer.
Embodiment 29. A method of inhibiting CD73, comprising contacting CD73 with a compound according to any one of embodiments 1-24, or a stereoisomer, tautomer, prodrug, or a pharmaceutically acceptable salt of any of the foregoing.
Embodiment 30. Use of a compound of any one of embodiments 1-24, or a stereoisomer, tautomer, prodrug, or a pharmaceutically acceptable salt of any of the foregoing, in the manufacture of a medicament for use in therapy.

### General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High-Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate.

Chromatography, recrystallization and other conventional separation procedures may also be used with intermediates or final products where it is desired to obtain a particular isomer of a compound or to otherwise purify a product of a reaction.

General methods of preparing compounds according to the present disclosure are depicted in the schemes below, wherein PG is a protective group; and X¹, X², X³, Y, Z, R¹, R², R³, R⁴, and R⁵ are as detailed herein.

As shown in Scheme 1, some compounds of this invention can be prepared from 1. Compounds of the general structure 1 are commercially available or can be prepared by procedures described in the literature. For example, the compound wherein Y═Z═CH, can be synthesized according to a procedure given in Journal of Natural Products, 51, 343, (1988). The compound wherein Y═CH and Z=C—F, can be synthesized according to a procedure given in PCT Int. Appl. (2012), WO 2012058671. The synthesis of compound 1, wherein Y═N and Z═CH is described in e.g. Bioorg. Med. Chem. Lett., 23, 2663, (2013).

Compound 1 can be converted into rhe dichloro compound 2 by reacting with SOCl₂, PCl₅ or POCl₃. A suitable base, such as PhNMe₂ can be added during the reaction. Elevated temperatures may be needed for the reaction to occur. Compound 3 can be prepared from 2 by reacting 2 with sodium methoxide. 3 can be treated with an organometallic compound to give the metalated species 4. This can be accomplished, for example, with n-BuLi, sec-BuLi or tert-Buli or with MeMgBr and ⁱPrMgBr in a solvent such as diethylether, dimethoxyethane, or THF. The organometallic species 4 can be added to the appropriately protected lactone 5, to give 6. Appropriate protecting groups (PG) are known to those skilled in the art and are described, for example, in “Greene's Protective Groups in Organic Synthesis”, John Wiley & Sons, Inc., 2014.

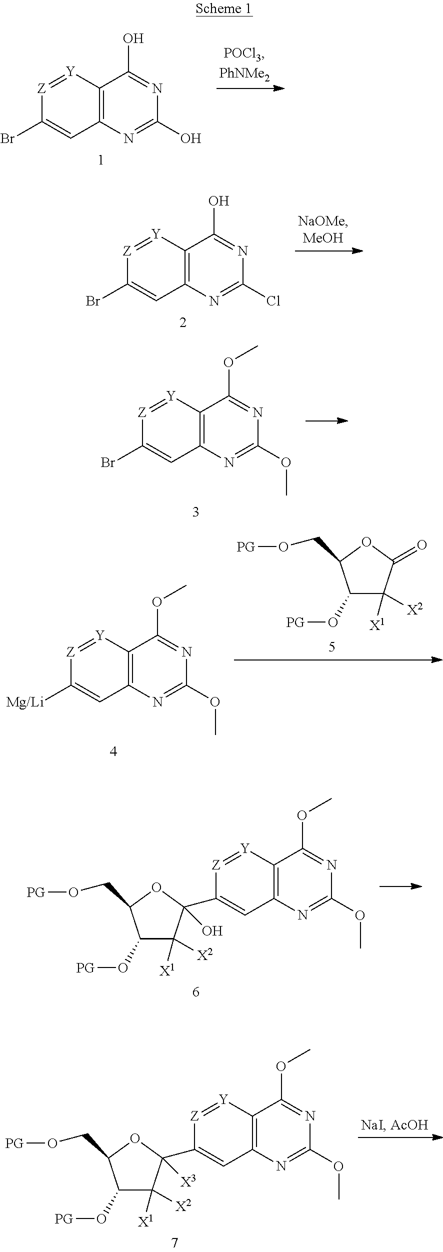

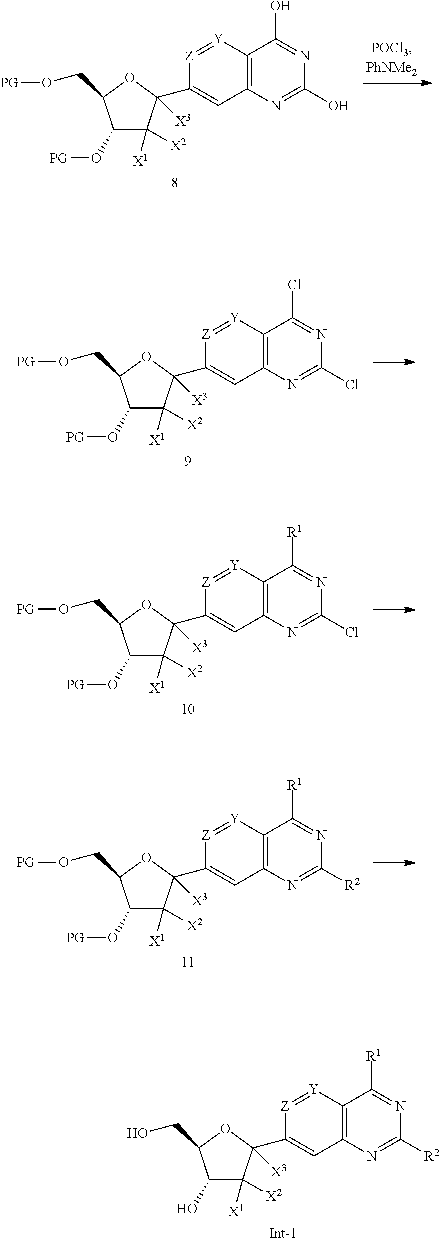

6 can be transformed into 7 by methods generally known to those skilled in the art. For example, if X³═H, Et₃SiH in the presence of a Lewis acid, such as BF₃.OEt₂ will accomplish this transformation. De-methylation of 7 to give 8 can be accomplished, for example, by NaI in AcOH. 8 can be converted into the chloro derivative 9 by reacting with e.g. SOCl₂, PCl₅ or POCl₃, as described above. 9 can be reacted with an alcohol in the presence of a base, such as sodium hydride in a solvent, such as THF, to give 10, wherein R¹═—OR^{1a}. Alternatively, 9 can be reacted with a primary or secondary amine in the presence of a base, such as, for example, Et₃N or DIEA in a solvent, such as THF or EtOH to give 10, wherein R¹═—NR^{1a}R^{1b}. 10 can be converted into 11 by methods described in the individual Examples below. 11 can be deprotected to give Int-1. Deprotection will be accomplished by methods known to the skilled practitioner and are also described in “Greene's Protective Groups in Organic Synthesis”, John Wiley & Sons, Inc., 2014. For example, if the protection group is a benzyl ether (PG=Bn), hydrogen in presence of a catalyst, such as Pd on carbon, or BCl₃ in DCM will achieve the deprotection. If the protection group is a silyl ether, the deprotection can be accomplished, for example, by using Bu₄NF in THF. Many other protecting groups and methods for removing them are known to those skilled in the art.

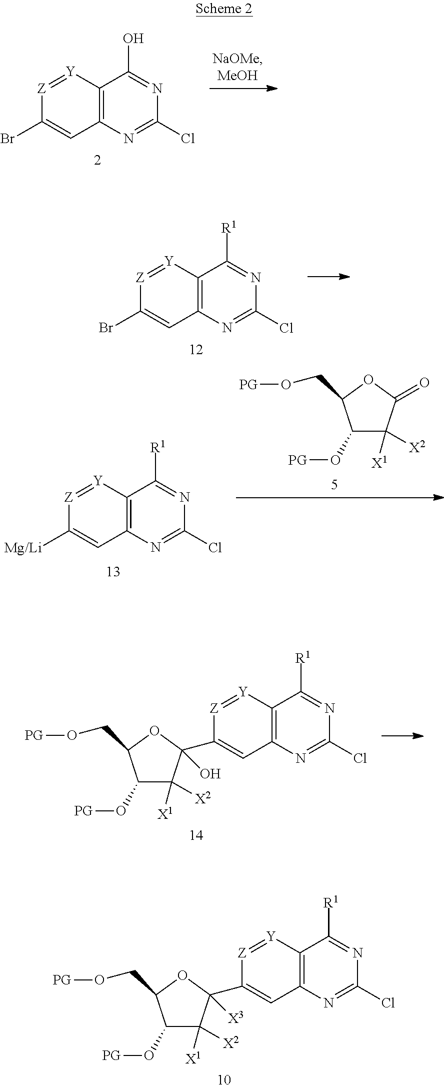

Scheme 2 shows an alternative synthesis of compound 10. Compound 12 can be prepared from 2 by reacting 2 with 1 eq. of sodium methoxide. 12 can be treated with an organometallic compound to give the metalated species 4. This can be accomplished, for example, with n-BuLi, sec-BuLi or tert-Buli or with MeMgBr and ¹PrMgBr in a solvent such as diethylether, dimethoxyethane, or THF. The organometallic species 4 can be added to the appropriately protected lactone 5, to give 14. 14 can be transformed into 10 by methods generally known to those skilled in the art. For example, if X³═H, Et₃SiH in the presence of a Lewis acid, such as BF₃.OEt₂ will accomplish this transformation. If X³═CN, Et₃SiCN in the presence of a Lewis acid, such as BF₃.OEt₂ can be used. The transformation to 10, wherein X³═Me, can be achieved, for example, by using AlMe₃ in a suitable solvent, such as toluene.

Compounds of formula 15a and 15b may be prepared according to steps outlined in Scheme 3. For example, reaction of Int-1 with methylenebis(phosphonic dichloride) followed by hydrolysis with a suitable base, such as TEAC, can provide 15a. Alternatively, reaction of Int-1 with methylenebis(phosphonic acid) or a suitable methylenebis(phosphonic acid) ester in presence of a coupling reagent, such as DCC, will provide 15b. Int-1 can also be converted into a mesylate, tosylate or triflate (16) by methods known to a person skilled in the art. Reaction of 16 with methylenebis(phosphonic acid) or a suitable methylenebis(phosphonic acid) ester in presence of a coupling reagent, such as DCC, will provide 15b which may be hydrolyzed to 15 a using an acid, such as formic acid or acetic acid.

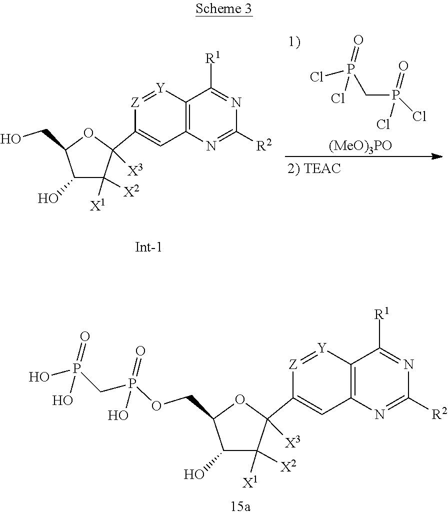

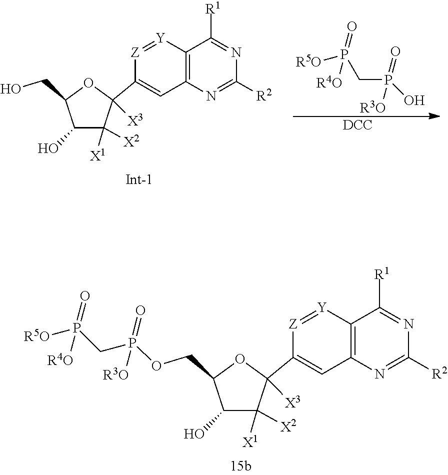

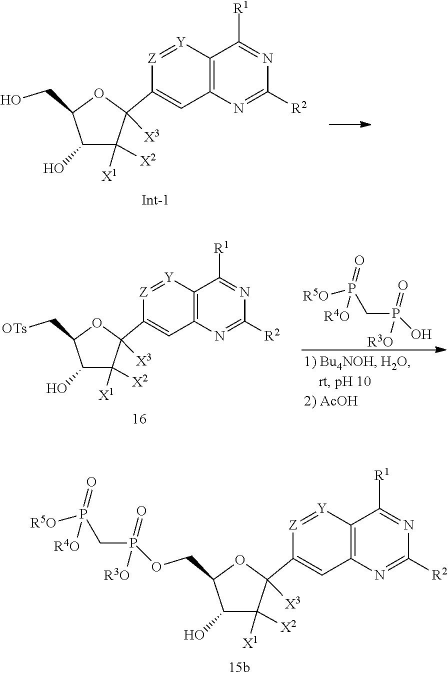

Scheme 4 below exemplifies the synthesis of compounds of the general structure 24. Briefly, di-tert-butyl phosphonate (17) is alkylated with MeI in presence of a base, such as NaH or BuLi, in a suitable solvent to give 18. Deprotonation of 18 with a base, such as LDA, followed by reaction with 1-chloro-N,N,N′,N′-tetraisopropylphosphanediamine (3), yields compound 19. One of the diisopropylamino groups can be displaced by an alcohol (R—OH) or by water (R═H) to give 20. Reaction of 20 with alcohol 21 in the presence of a coupling reagent, such as DCI in a suitable solvent, such as ACN will furnish 22. 22 can be oxidized to 23 by an organic peroxide, such as, for example, tert-butyl hydroperoxide. Hydrolysis of the tert-butyl ester groups of 23 under acidic conditions and removal of the protecting group PG will furnish 24. Appropriate protecting groups (PG) are known to those skilled in the art and their introduction and removal are described, for example, in “Greene's Protective Groups in Organic Synthesis”, John Wiley & Sons, Inc., 2014.

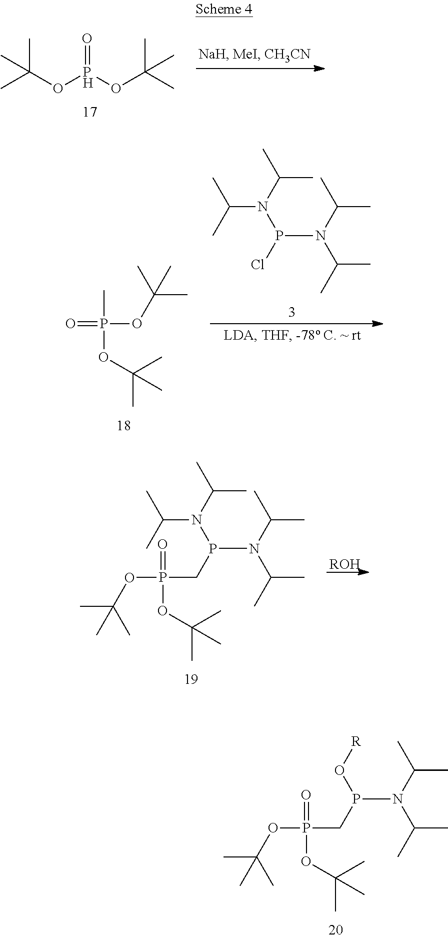

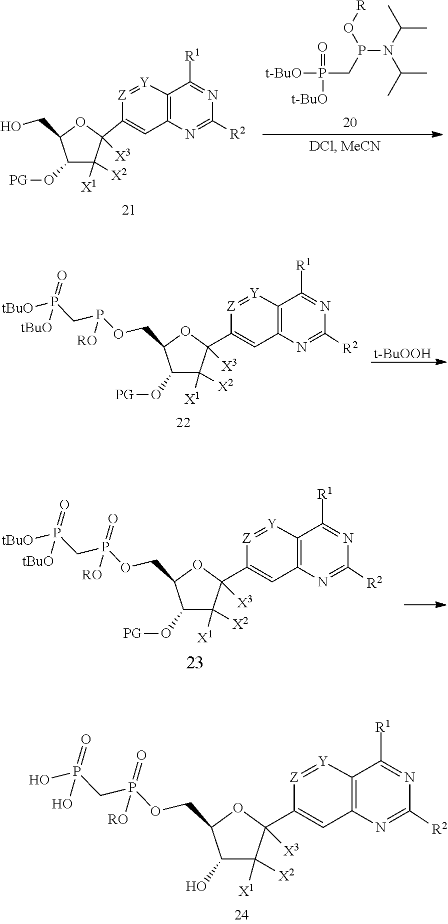

In some embodiments a compound of the present invention, for example a compound of a formula given in Table 1, is synthesized according to one of the general routes outlined in Schemes 1-4, Examples S1-S46 or by methods generally known to those skilled in the art.

### EXAMPLES

It is understood that the present disclosure has been made only by way of example, and that numerous changes in the combination and arrangement of parts can be resorted to by those skilled in the art without departing from the spirit and scope of present disclosure.

The chemical reactions in the Examples described can be readily adapted to prepare a number of other compounds discsimilar to the oneslosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

The following abbreviations may be used herein:

about
+ve or pos. ion positive ion
Δ heat
Ac Acetyl
ACN Acetonitrile
Ac₂O acetic anhydride
AcOH acetic acid
AMP Adenosine monophosphate
anh. anhydrous
aq aqueous
Bn benzyl
Boc tert-butyloxycarbonyl
BSA bovine serum albumin
Bz benzoyl
Calcd or Calc'd calculated
CombiFlash® CombiFlash®, Teledyne ISCO Inc., Lincoln Nebr., USA
Conc. concentrated
d day(s) or doublet (NMR)
DCC dicyclohexylcarbodiimide
DCE dichloroethane
DCI 1H-Imidazole-4,5-dicarbonitrile
DCM dichloromethane
dd Dublet of doublets (NMR)
DEA diethylamine
DIEA or DIPEA diisopropylethylamine
DME 1,2-dimethoxyethane
DMF N,N-dimethylformamide
DMSO dimethyl sulfoxide
EA Ethyl acetate
eq equivalent
ESI electrospray ionization
Et ethyl
Et₂O diethyl ether
Et₃N triethylamine
EtOAc ethyl acetate
EtOH ethyl alcohol
FA formic acid
g gram(s)
h hour(s)
Hex hexanes
RMPA hexamethylphosphoramide
HPLC high performance liquid chromatography
Hz Hertz
IPA or iPrOH isopropyl alcohol
J coupling constant (NMR) in Hz
KOAc potassium acetate
LCMS, LC-MS or LC/MS liquid chromatography mass spectrometry
LDA lithium diisopropylamide
LHMDS or LiHMDS lithium hexamethyldisilazide
m multiplet (NMR)
M molar (mol L⁻¹)
Me methyl
MeCN acetonitrile
MeI iodomethane
MeOH methyl alcohol
mg milligram(s)
min minute(s)
mL milliliter(s)
M mole(s)
MS mass spectrometry
MSCl methanesulfonyl chloride
MTBE or MtBE methyl tert-butyl ether
m/z mass-to-charge ratio
NaHMDS sodium hexamethyldisilazide
NaOtBu sodium tert-butoxide
nBuLi n-butyl lithium
NCS N-chloro succinimide
nm nanometer (wavelength)
NMR nuclear magnetic resonance
P1 Product one; faster eluting isomer
P2 Product two; slower eluting isomer
PCC Pyridinium chlorochromate, CAS Number: 26299-14-9
PE Petroleum ether, CAS Number: 101316-46-5
PBS phosphate buffered saline
PMB para-methoxybenzyl, 4-methoxybenzyl
Pr propyl
ppm parts per million
p-tol para-toluoyl
rac racemic
RP-HPLC or RPHPLC reversed phase high performance liquid chromatography
RT or rt or r.t. room temperature
s singlet (NMR)
sat. or sat'd or satd saturated
SFC Supercritical fluid chromatography
t triplet (NMR)
TBSCl tert-Butyldimethylsilyl chloride
tBuOH tert-butyl alcohol
TEA triethylamine
TEAC tetraethylammonium chloride
tert or t tertiary
TFA triflouroacetic acid
THF tetrahydrofuran
TLC thin layer chromatography
TMS trimethylsilyl or trimethylsilane
Tris tris(hydroxymethyl)aminomethane
v/v volume per volume

### Example S1

### Synthesis of (((((2R,3S,4R,5S)-5-(4-(benzylamino)-2-chloroquinazolin-7-yl)-3,4-dihydroxytetrahydrofuran-2-yl)methoxy)(hydroxy)phosphoryl)methyl)phosphonic acid

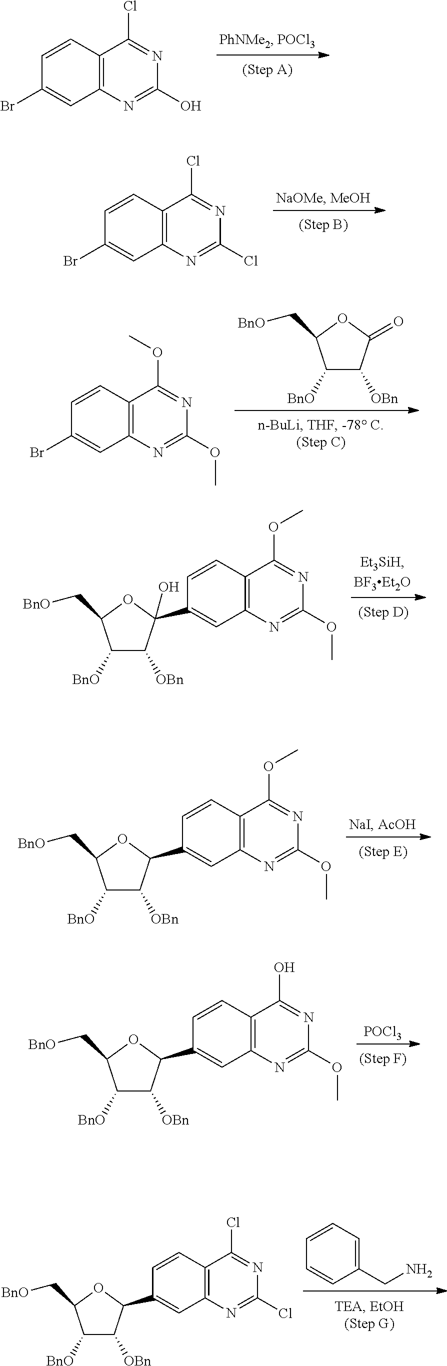

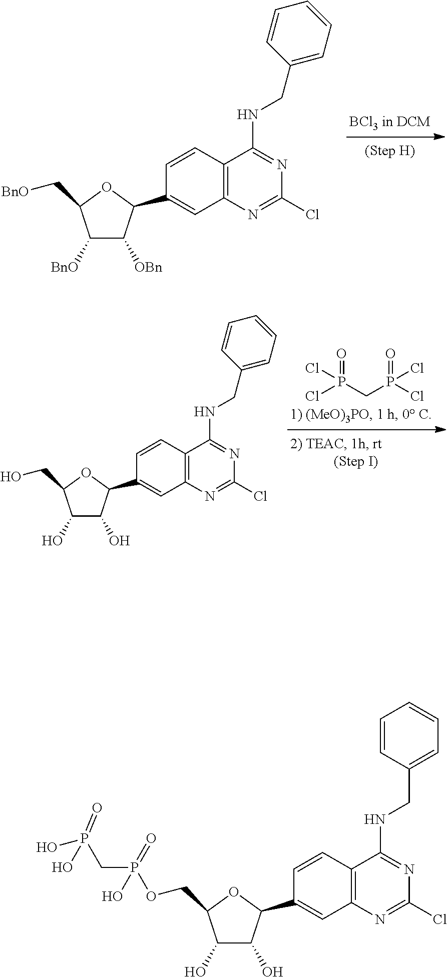 [SMILES: Clc1nc(NCc2ccccc2)c2ccc([C@@H]3O[C@H](COCc4ccccc4)[C@@H](OCc4ccccc4)[C@H]3OCc3ccccc3)cc2n1 ; "BCl3 in DCM" ; OC[C@H]1O[C@@H](c2ccc3c(NCc4ccccc4)nc(Cl)nc3c2)[C@H](O)[C@@H]1O ; O=P(Cl)(Cl)CP(=O)(Cl)Cl ; O=P(O)(O)CP(=O)(O)OC[C@H]1O[C@@H](c2ccc3c(NCc4ccccc4)nc(Cl)nc3c2)[C@H](O)[C@@H]1O]

Step A: To a solution of 7-bromoquinazoline-2,4-diol (20 g, 83 mmol) in phosphorus oxychloride (100 mL) was added N,N-dimethylaniline (20.1 g. 166 mmol). The reaction mixture was refluxed for 4 h. The solvent was removed under reduced pressure and the residue was dropped into ice water carefully until phosphorus oxychloride was quenched completely, then extracted with ethyl acetate (100 mL×3). The combined organic phases were concentrated and purified by silica gel column chromatography (PE/EA=5:1) to give 7-bromo-2,4-dichloroquinazoline (17.36 g, 75.5% yield) as a white solid. Mass Spectrum (ESI) m/z=277 (M+1).

Step B: To a solution of 2,4-dichlorofuro[3,2-d]pyrimidine (17.36 g, 63 mmol) in MeOH (300 mL) was added sodium methoxide (20.40 g, 378 mmol). The reaction mixture was refluxed for 4 h. The mixture was filtered, the filtrate was concentrated under reduced pressure, and the residue was purified by silica gel column chromatography (PE/EA=4:1) to obtain 7-bromo-2,4-dimethoxyquinazoline (14.13 g, 79.4% yield) as a white solid. Mass Spectrum (ESI) m/z=269 (M+1).

Step C: To a solution of 7-bromo-2,4-dimethoxyquinazoline (14.13 g, 53 mmol) in 200 mL anhydrous THF was carefully added n-BuLi (2.4 M, 28.5 mL, 69 mmol) dropwise at −78° C. under a nitrogen atmosphere. The reaction mixture was stirred for 30 min at −78° C., then a solution of (3R,4R,5R)-3,4-bis(benzyloxy)-5-[(benzyloxy) methyl]oxolan-2-one (22.05 g, 53 mmol) in 40 mL anhydrous THF was added dropwise over 30 min. The reaction was stirred for 2 h at −78° C., then for 2 h at −30° C. After quenching with sat. aq. NH₄Cl solution in cold, the mixture was extracted with EA, the organic layer was dried over anhydrous Na₂SO₄, filtered, and the filtrate was concentrated uder reduced pressure. The dried, crude product was purified by silica gel column chromatography (PE/EA=3:1) to give (2S,3R,4R,5R)-3,4-bis(benzyloxy)-5-[(benzyloxy)methyl]-2-(2,4-dimethoxyquinazolin-7-yl)oxolan-2-ol (21.4 g, 66.7% yield) as a light yellow solid. Mass Spectrum (ESI) m/z=609 (M+1).

Step D: (2S,3R,4R,5R)-3,4-bis(benzyloxy)-5-[(benzyloxy)methyl]-2-(2,4-dimethoxyquinazolin-7-yl)oxolan-2-ol (21.4 g, 35 mmol) was dissolved in 200 mL anhydrous CH₂Cl₂ under nitrogen and stirred at −78° C. To this mixture, triethylsilane (16.33 g, 140 mmol) was added dropwise, followed by boron trifluoride diethyl etherate (48%, 41.4 g, 140 mmol). The reaction was stirred overnight at −78° C. and allowed to warm up to room temperature. After quenching with sat. aq. NaHCO₃ solution, the organic layer was dried over anhydrous Na₂SO₄, filtered, and the filtrate was evaporated in vacuo. The residue was purified by silica gel column chromatography (PE/EA=5:1) to give 7-[(2S,3S,4R,5R)-3,4-bis(benzyloxy)-5-[(benzyloxy)methyl]oxolan-2-yl]-2,4-dimethoxyquinazoline(18.75 g, 90% yield) as a light yellow solid. Mass Spectrum (ESI) m/z=593 (M+1).

Step E: A mixture of 7-[(2S,3S,4R,5R)-3,4-bis(benzyloxy)-5-[(benzyloxy)methyl]oxolan-2-yl]-2,4-dimethoxyquinazoline (18.75 g, 32 mmol) and sodium iodide (23.98 g, 160 mmol) in AcOH (200 mL) was stirred at 60° C. for 45 min. The reaction mixture was concentrated under reduced pressure. The residue was dissolved in DCM and the organic layer was washed with sat. aq. Na₂S₂O₄ solution and sat. aq. NaHCO₃ solution. The combined aqueous layers were extracted with DCM. The combined organic layers were concentrated and purified by silica gel column chromatography (PE/EA=1:3) to give 7-[(2S,3S,4R,5R)-3,4-bis(benzyloxy)-5-[(benzyloxy)methyl]oxolan-2-yl]quinazoline-2,4-diol (16.97 g, 95% yield) as a yellow oil. Mass Spectrum (ESI) m/z=565 (M+1).

Step F: To a solution of 7-[(2S,3S,4R,5R)-3,4-bis(benzyloxy)-5-[(benzyloxy)methyl]oxolan-2-yl]quinazoline-2,4-diol (16.97 g, 3 mmol) in phosphorus oxychloride (200 mL) was added N,N-dimethylaniline (7.27 g. 6 mmol) and the reaction mixture was stirred and heatd to reflux for 4 h. The solvent was removed under reduced pressure and the residue was dropped into ice water carefully until phosphorus oxychloride was quenched completely, then extracted with ethyl acetate (100 mL×3). The combined organic phases were concentrated and purified by silica gel column chromatography (PE/EA=4:1) to get 7-[(2S,3S,4R,5R)-3,4-bis(benzyloxy)-5-[(benzyloxy)methyl]oxolan-2-yl]-2,4-dichloroquinazoline (15.34 g, 85% yield) as a white solid. Mass Spectrum (ESI) m/z=601 (M+1).

Step G: To a solution of 7-[(3S,4R,5R)-3,4-bis(benzyloxy)-5-[(benzyloxy)methyl]oxolan-2-yl]-2,4-dichloroquinazoline (370 mg, 0.62 mmol) in ethanol (8 mL) was added benzylamine (99 mg, 0.92 mmol) and TEA (0.26 mL, 1.86 mmol). The resulting reaction was heated to reflux for 4 h. The solvent was removed and the residue was purified by CombiFlash® (eluting with PE/EA=1:1) to give N-benzyl-7-[(3S,4R,5R)-3,4-bis(benzyloxy)-5-[(benzyloxy)methyl]oxolan-2-yl]-2-chloroquinazolin-4-amine (320 mg, 77.4% yield) as a yellow solid. Mass Spectrum (ESI) m/z=672.1 (M+1).

Step H: To a solution of N-benzyl-7-[(3S,4R,5R)-3,4-bis(benzyloxy)-5-[(benzyloxy)methyl]oxolan-2-yl]-2-chloroquinazolin-4-amine (320 mg, 0.48 mmol) in DCM (5 mL) was added BCl₃ (1 M in DCM, 4.8 mL, 4.8 mmol) dropwise at −70° C. under an N₂ atmosphere. Then the reaction solution was stirred at −70° C. for 1 h. Then the reaction was brought to −30° C. over a period of 30 min, and quenched by adding a mixture of methanol:chloroform (2:1, 10 mL). The reaction mixture was allowed to warm to rt, then it was neutralized with NH₃ in methanol (10%, 10 mL) and concentrated. The residue was purified by CombiFlash® (4 g, eluting with DCM/MeOH/NH₄OH=70:30:5) to give the crude product which was resolved with DCM/MeOH (10:1) and filtered. The filtrate was concentrated to give (3R,4S,5R)-2-[4-(benzylamino)-2-chloroquinazolin-7-yl]-5-(hydroxymethyl)oxolane-3,4-diol (200 mg,87.50%) as a yellow solid. Mass Spectrum (ESI) m/z=402.1 (M+1).

Step I: To a solution of (3R,4S,5R)-2-[4-(benzylamino)-2-chloroquinazolin-7-yl]-5-(hydroxymethypoxolane-3,4-diol (100 mg,0.25 mmol) in trimethyl phosphate (2 mL) at 0° C. was added a cold solution of methylenebis(phosphonic dichloride) (309 mg, 1.25 mmol) in trimethyl phosphate (1 mL) dropwise. Then the reaction solution was stirred at 0° C. for 1 h. TEAC (0.5 M, 1.75 mL) was added to the reaction carefully and the reaction was stirred at this temperature for 15 mins, then warmed to room temperature and continued to stir for 1 h. Trimethyl phosphate was extracted using tert-butyl methyl ether (5 mL×2) and the aqueous layer was basified with ammonium hydroxide to pH ˜7-8, then the solution was purified by Prep-HPLC using a gradient of 0.2% ammonium hydroxide in water/ACN from 100:0 to 85:15, and suitable fractions were pooled and lyophilized to give the final product [({[(2R,3S,4R,5S)-5-[4-(benzylamino)-2-chloroquinazolin-7-yl]-3,4-dihydroxyoxolan-2-yl]methoxy}(hydroxy)phosphoryl)methyl]phosphonic acid (25 mg, 16%) as a white solid.

¹H NMR (400 MHz, D₂O) δ7.93 (d, J=8.4 Hz, 1H), 7.61 (d, J=8.7 Hz, 1H), 7.49 (s, 1H), 7.38-7.17 (m, 5H), 4.87-4.83 (m, 3H), 4.28-4.20 (m, 2H), 4.14-4.04 (m, 3H), 2.11 (t, J=19.8 Hz, 2H). Mass Spectrum (ESI) m/z=560.0 (M+1).

### Example S2

### Synthesis of (((((2R,3S,4R,5S)-5-(2-chloro-4-(phenethylamino)quinazolin-7-yl)-3,4-dihydroxytetrahydrofuran-2-yl)methoxy)(hydroxy)phosphoryl)methyl)phosphonic acid

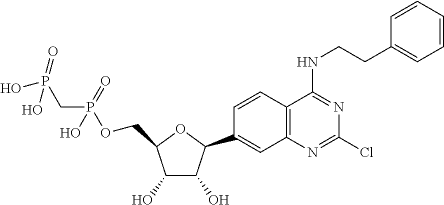 [SMILES: O=P(O)(O)CP(=O)(O)OC[C@H]1O[C@@H](c2ccc3c(NCCc4ccccc4)nc(Cl)nc3c2)[C@H](O)[C@@H]1O]

(((((2R,3S,4R,5S)-5-(2-chloro-4-(phenethylamino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with 2-phenylethan-1-amine.

¹H NMR (400 MHz, DMSO-d6) δ ppm 8.86-8.84 (m, 1H), 8.21 (d, J=9.0 Hz, 1H), 7.62-7.56 (m, 2H), 7.31-7.22 (m, 5H), 4.75 (d, J=6.8 Hz, 1H), 4.14-3.95 (m, 5H), 3.76-7.72 (m, 2H), 3.02-2.94 (m, 2H), 2.32-2.16 (m, 2H). Mass Spectrum (ESI) m/z=573. 8 (M+1).

### Example S3

### Synthesis of (((((2R,3S,4R,5S)-5-(2-chloro-4-(cyclopentyl(methyl)amino)quinazolin-7-yl)-3,4-dihydroxytetrahydrofuran-2-yl)methoxy)(hydroxy)phosphoryl)methyl)phosphonic acid

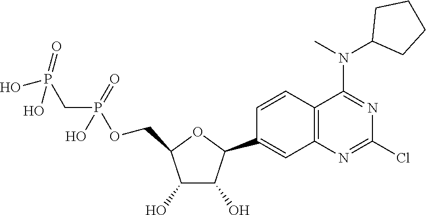 [SMILES: CN(c1nc(Cl)nc2cc([C@@H]3O[C@H](COP(=O)(O)CP(=O)(O)O)[C@@H](O)[C@H]3O)ccc12)C1CCCC1]

(((((2R,3S,4R,5S)-5-(2-chloro-4-(cyclopentyl(methyl)amino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with N-methylcyclopentanamine.

¹H NMR (400 MHz, DMSO-d6) δ 8.11 (d, J=8.8 Hz, 1H), 7.61 (s, 1H), 7.55 (d, J=8.9 Hz, 1H), 4.93-4.83 (m, 1H), 4.75 (d, J=6.9 Hz, 1H), 4.15-4.05 (m, 3H), 4.04-3.96 (m, 1H), 3.83-3.75 (m, 1H), 3.21 (s, 3H), 2.24 (t, J=20.1 Hz, 2H), 2.05-1.95 (m, 2H), 1.80-1.67 (m, 4H), 1.65-1.55 (m, 2H). Mass Spectrum (ESI) m/z=550.0 (M−1).

### Example S4

### Synthesis of (((((2R,3S,4R,5S)-5-(2-chloro-4-(cyclopentylamino)quinazolin-7-yl)-3,4-dihydroxytetrahydrofuran-2-yl)methoxy)(hydroxy)phosphoryl)methyl)phosphonic acid

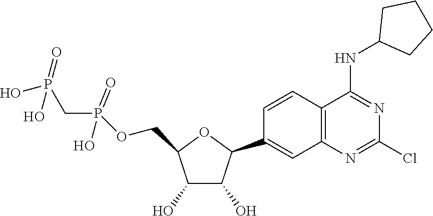 [SMILES: O=P(O)(O)CP(=O)(O)OC[C@H]1O[C@@H](c2ccc3c(NC4CCCC4)nc(Cl)nc3c2)[C@H](O)[C@@H]1O]

(((((2R,3S,4R,5S)-5-(2-chloro-4-(cyclopentylamino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with cyclopentanamine.

¹H NMR (400 MHz, DMSO-d6+D₂O) δ 8.44-8.30 (m, 1H), 7.73-7.78 (m, 2H), 4.77-4.67 (m, 1H), 4.53-4.42 (m, 1H), 4.11-3.91 (m, 3H), 3.83-3.75 (m, 1H), 3.14-3.03 (m, 1H), 2.05-1.92 (m, 2H), 1.83-1.68 (m, 2H), 1.68-1.39 (m, 4H), 1.22-1.12 (m, 2H). Mass Spectrum (ESI) m/z=536.0 (M−1).

### Example S5

### Synthesis of (((((2R,3S,4R,5S)-5-(2-chloro-4-((cyclopentylmethyl)amino)quinazolin-7-yl)-3,4-dihydroxytetrahydrofuran-2-yl)methoxy)(hydroxy)phosphoryl)methyl)phosphonic acid

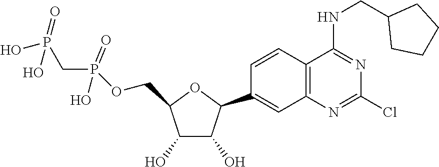 [SMILES: O=P(O)(O)CP(=O)(O)OC[C@H]1O[C@@H](c2ccc3c(NCC4CCCC4)nc(Cl)nc3c2)[C@H](O)[C@@H]1O]

(((((2R,3S,4R,5S)-5-(2-chloro-4-((cyclopentylmethyl)amino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with cyclopentylmethanamine.

¹H NMR (400 MHz, D₂O) δ 8.06 (d, J=8.6 Hz, 1H), 7.89-7.83 (m, 1H), 7.61 (d, J=8.3 Hz, 1H), 4.90 (d, J=6.2 Hz, 1H), 4.28-4.20 (m, 2H), 4.13-4.05 (m, 3H), 3.58 (d, J=7.1 Hz, 2H), 2.30-2.13 (m, 3H), 1.72-1.65 (m, 2H), 1.58-1.52 (m, 2H), 1.48-1.43 (m, 2H), 1.25-1.18 (m, 2H). Mass Spectrum (ESI) m/z=550 (M−1).

### Example S6

### Synthesis of (((((2R,3S,4R,5S)-5-(2-chloro-4-(((tetrahydrofuran-2-yl)methyl)amino)quinazolin-dihydroxytetrahydrofuran-2-yl)methoxy)(hydroxy)phosphoryl)methyl)phosphonic acid

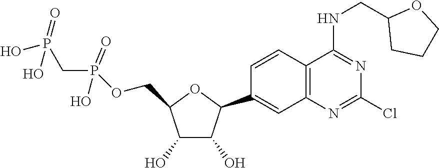 [SMILES: O=P(O)(O)CP(=O)(O)OC[C@H]1O[C@@H](c2ccc3c(NCC4CCCO4)nc(Cl)nc3c2)[C@H](O)[C@@H]1O]

(((((2R,3S,4R,5S)-5-(2-chloro-4-(((tetrahydrofuran-2-yl)methyl)amino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with (tetrahydrofuran-2-yl)methanamine.

¹H NMR (400 MHz, D₂O) δ 8.06 (d, J=8.6 Hz, 1H), 7.85 (s, 1H), 7.61 (d, J=8.5 Hz, 1H), 4.89 (d, J=6.8 Hz, 1H), 4.36-4.16 (m, 3H), 4.16-3.99 (m, 3H), 3.83-3.65 (m, 4H), 2.19 (t, J=18.5 Hz, 2H), 1.96-2.05 (m, 1H), 1.91-1.77 (m, 2H), 1.66-1.55 (m, 1H). Mass Spectrum (ESI) m/z=552.0 (M−1).

### Example S7

### Synthesis of (((((2R,3S,4R,5S)-5-(2-chloro-4-(cyclobutylamino)quinazolin-7-yl)-3,4-dihydroxytetrahydrofuran-2-yl)methoxy)(hydroxy)phosphoryl)methyl)phosphonic acid

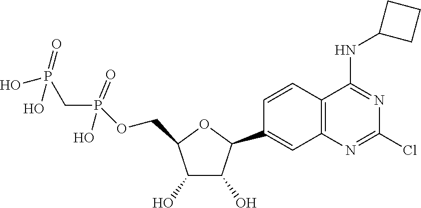 [SMILES: O=P(O)(O)CP(=O)(O)OC[C@H]1O[C@@H](c2ccc3c(NC4CCC4)nc(Cl)nc3c2)[C@H](O)[C@@H]1O]

(((((2R,3S,4R,5S)-5-(2-chloro-4-(cyclobutylamino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with cyclobutanamine.

¹H NMR (400 MHz, D₂O) δ 8.07 (d, J=8.6 Hz, 1H), 7.78 (s, 1H), 7.60 (d, J=7.4 Hz, 1H), 4.90 (d, J=6.6 Hz, 1H), 4.68 (s, 1H), 4.30-4.203 (m, 2H), 4.18-4.04 (m, 3H), 2.415-2.312 (m, 2H), 2.21-2.01 (m, 4H), 1.804-1.75 (m, J=5.6 Hz, 2H). Mass Spectrum (ESI) m/z=523.6 (M+1).

### Example S8

### Synthesis of (((((2R,3S,4R,5S)-5-(2-chloro-4-((cyclobutylmethyl)amino)quinazolin-7-yl)-3,4-dihydroxytetrahydrofuran-2-yl)methoxy)(hydroxy)phosphoryl)methyl)phosphonic acid

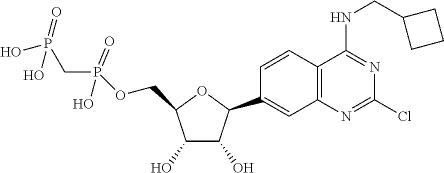 [SMILES: O=P(O)(O)CP(=O)(O)OC[C@H]1O[C@@H](c2ccc3c(NCC4CCC4)nc(Cl)nc3c2)[C@H](O)[C@@H]1O]

(((((2R,3S,4R,5S)-5-(2-chloro-4-(cyclobutylamino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with cyclobutylmethanamine.

¹H NMR (400 MHz, D₂O) δ 8.05 (d, J=8.6 Hz, 1H), 7.87 (s, 1H), 7.61 (d, J=8.5 Hz, 1H), 4.91 (d, J=6.7 Hz, 1H), 4.28-4.21 (m, 2H), 4.16-4.06 (m, 3H), 3.69 (d, J=6.0 Hz, 2H), 2.70-2.61 (m, 1H), 2.25-2.13 (m, 2H), 1.98 (t, J=19.2, 9.6 Hz, 2H), 1.83-1.67 (m, 4H). Mass Spectrum (ESI) m/z=537.7 (M+1).

### Example S9

### Synthesis of (((((2R,3S,4R,5S)-5-(2-chloro-4-((cyclopropylmethyl)amino)quinazolin-7-yl)-3,4-dihydroxytetrahydrofuran-2-yl)methoxy)(hydroxy)phosphoryl)methyl)phosphonic acid

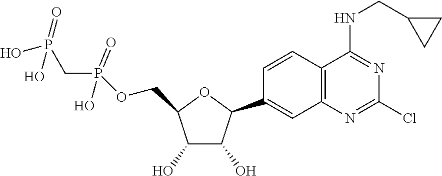 [SMILES: O=P(O)(O)CP(=O)(O)OC[C@H]1O[C@@H](c2ccc3c(NCC4CC4)nc(Cl)nc3c2)[C@H](O)[C@@H]1O]

(((((2R,3S,4R,5S)-5-(2-chloro-4-((cyclopropylmethyl)amino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with cyclopropylmethanamine.

¹H NMR (400 MHz, D₂O) δ 8.07 (d, J=8.7 Hz, 1H), 7.88 (s, 1H), 7.60 (d, J=8.7 Hz, 1H), 4.91-4.87 (m, 1H), 4.57-4.56 (m, 1H), 4.27-4.24 (m, 1H), 4.23-4.20 (m, 1H), 4.13-4.07 (m, 2H), 3.48 (d, J=7.1 Hz, 2H), 2.20-2.07 (m, 2H), 1.17-1.12 (m, 1H), 0.52-0.44 (m, 2H), 0.28-0.20 (m,2H). Mass Spectrum (ESI) m/z=524.0 (M+1).

### Example S10

### Synthesis of (((((2R,3S,4R,5S)-5-(4-(cyclopentyl(methyl)amino)quinazolin-7-yl)-3,4-dihydroxytetrahydrofuran-2-yl)methoxy)(hydroxy)phosphoryl)methyl)phosphonic acid

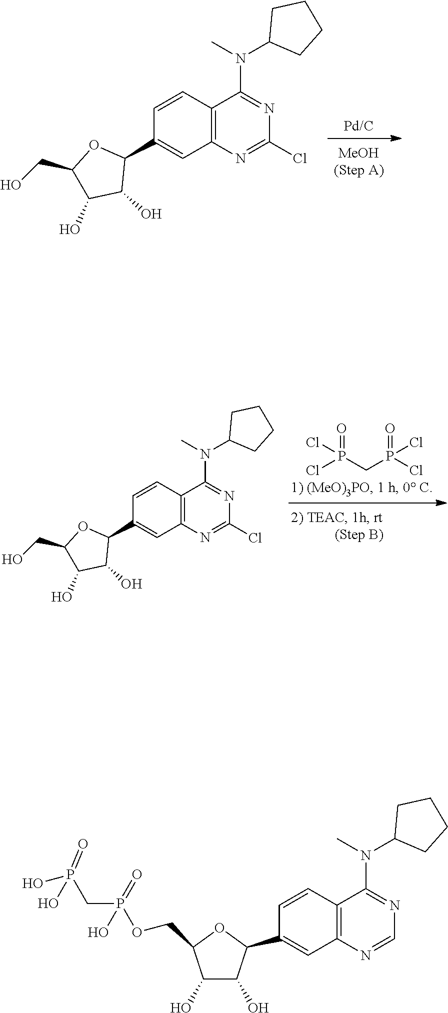

Step A: A mixture of (2S,3R,4S,5R)-2-(2-chloro-4-(cyclopentyl(methyl)amino)quinazolin-7-yl)-5-(hydroxymethyl)tetrahydrofuran-3,4-diol (220 mg, 0.56 mmol) and Pd/C (20 mg) in CH₃OH (6 mL) was stirred under a H₂ atmosphere for 1 h at room temperature. The mixture was filtered and the filtrate was concentrated and purified by CombiFlash® (DCM/MeOH=10:1) to afford (2S,3S,4S,5R)-2-{2-chloro-4-[cyclopentyl(methyl)amino]quinazolin-7-yl}-5-(hydroxymethyl)oxolane-3,4-diol as a brown oil (170 mg, 68% yield). Mass Spectrum (ESI) m/z=394.1 (M+1).

Step B: To a solution of (2S,3S,4S,5R)-2-{2-chloro-4-[cyclopentyl(methyl)amino]quinazolin-7-yl}-5-(hydroxymethyl)oxolane-3,4-diol (170 mg, 0.43 mmol) in trimethyl phosphate (1 mL) at 0° C. was added a cold solution of methylenebis(phosphonic dichloride) (536 mg, 2.15 mmol) in trimethyl phosphate (1 mL) dropwise. Then the reaction solution was stirred at 0° C. for 1 h. TEAC (0.5 M, 3 mL) was added to the reaction carefully and the reaction was stirred at this temperature for 15 mins, then warmed to room temperature and continued to stir for 1 h. Trimethyl phosphate was extracted using tert-butyl methyl ether (5 mL×3), and the aqueous layer was basified with ammonium hydroxide to pH ˜7-8. The solution was purified by Prep-HPLC using a gradient of 0.2% formic acid in water/ACN from 90:10 to 50:50, and suitable fractions were pooled and lyophilized to give (((((2R,3S,4R,5S)-5-(4-(cyclopentyl(methyl)amino)quinazolin-7-yl)-3,4-dihydroxytetrahydrofuran-2-yl)methoxy)(hydroxy)phosphoryl)methyl)phosphonic acid (20 mg, 44% yield).

¹H NMR (400 MHz, DMSO-d6) δ 8.47 (s, 1H), 8.30-8.13 (m, 1H), 8.07 (s, 1H), 7.64 (d, J=8.6 Hz, 1H), 5.03 (d, J=6.2 Hz, 1H), 4.44-4.08 (m, 6H), 2.26 (t, J=19.6 Hz, 2H), 2.14-2.01 (m, 3H), 1.96-1.75(m, 4H), 1.74-1.60 (m, 2H). Mass Spectrum (ESI) m/z=518.0 (M+1).

### Example S11

### Synthesis of [({[(2R,3R,4S,5S)-5-[4-(benzylamino)quinazolin-7-yl]-4-fluoro-3-hydroxyoxolan-2-yl]methoxy}(hydroxy)phosphoryl)methyl]phosphonic acid

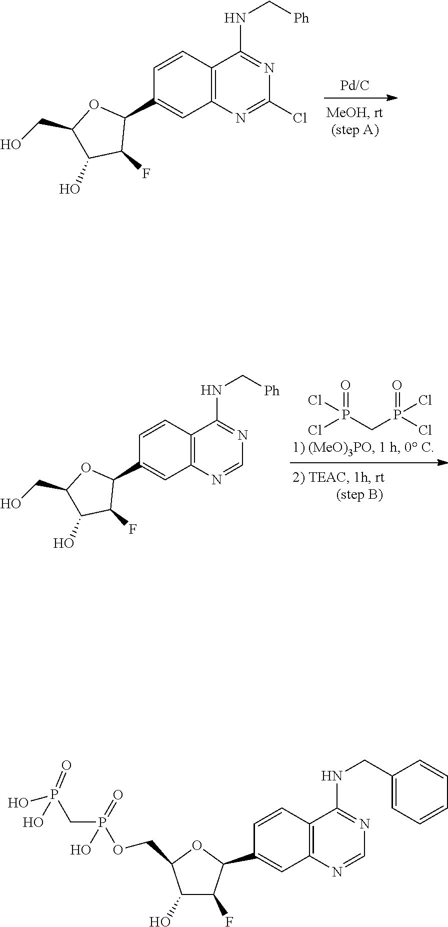 [SMILES: OC[C@H]1O[C@@H](c2ccc3c(NCc4ccccc4)nc(Cl)nc3c2)[C@@H](F)[C@@H]1O ; OC[C@H]1O[C@@H](c2ccc3c(NCc4ccccc4)ncnc3c2)[C@@H](F)[C@@H]1O ; O=P(Cl)(Cl)CP(=O)(Cl)Cl ; O=P(O)(O)CP(=O)(O)OC[C@H]1O[C@@H](c2ccc3c(NCc4ccccc4)ncnc3c2)[C@@H](F)[C@@H]1O]

Step A: To a solution of (2R,3R,4S)-5-[4-(benzylamino)-2-chloroquinazolin-7-yl]-4-fluoro-2-(hydroxymethyl)oxolan-3-ol (120 mg, 0.30 mmol) in MeOH (5 mL) was added 10% Pd/C (100 mg). The reaction was stirred at rt for 1.5 h under a H₂ atmosphere. The reaction was filtered and the filtrate was concentrated in vacuo to give (2R,3R,4S)-5-[4-(benzylamino)quinazolin-7-yl]-4-fluoro-2-(hydroxymethyl)oxolan-3-ol (100 mg, 75% yield) as an off-white solid. Mass Spectrum (ESI) m/z=370.1 (M+1).

Step B: To a solution of (2R,3R,4S)-5-[4-(benzylamino)quinazolin-7-yl]-4-fluoro-2-(hydroxymethypoxolan-3-ol (100 mg, 0.27 mmol) in Trimethyl phosphate (1.5 mL) was added a cold solution of methylenebis(phosphonic dichloride) (337 mg, 1.35 mmol) in trimethyl phosphate (1.5 mL) dropwise at 0° C. The reaction was stirred for 4 h. TEAC (0.5 M, 1.73 mL) was added to the reaction carefully and the reaction was stirred at this temperature for 15 mins, then warmed to room temperature and continued to stir for 1 h. Trimethyl phosphate was extracted using tert-butyl methyl ether (5 mL×2) and the aqueous layer was basified with ammonium hydroxide to pH ˜7-8. Then the solution was purified by Prep-HPLC using a gradient of 0.2% formic acid in water/ACN from 90:10 to 75:25, and suitable fractions were pooled and lyophilized to give [({[(2R,3R,4S,5S)-5-[4-(benzylamino)quinazolin-7-yl]-4-fluoro-3-hydroxyoxolan-2-yl]methoxy}(hydroxy)phosphoryl)methyl]phosphonic acid (22 mg, 14% yield) as an off-white solid.

¹H NMR (400 MHz, DMSO-d6) δ 9.98 (s, 1H), 8.84-8.68 (m, 1H), 8.53-8.42 (m, 1H), 7.89-7.68 (m, 2H), 7.42-7.23 (m, 5H), 5.39-5.31 (m, 1H), 5.15-5.03 (m, 1H), 4.98-4.86 (m, 2H), 4.43-4.25 (m, 1H), 4.24-4.17 (m, 1H), 4.15-3.83 (m, 2H), 2.24-2.03 (m, 2H). Mass Spectrum (ESI) m/z=527.7 (M+1).

### Example S12

### Synthesis of [({[(2R,3S,4R,5S)-5-[4-(benzylamino)quinazolin-7-yl]-3,4-dihydroxyoxolan-2-yl]methoxy}(hydroxy)phosphoryl)methyl]phosphonic acid

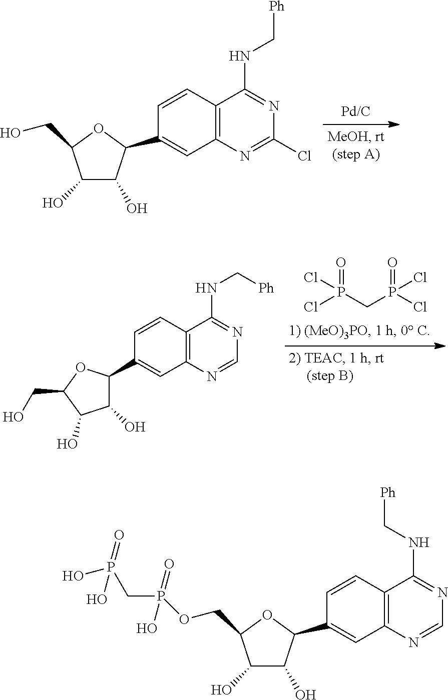 [SMILES: OC[C@H]1O[C@@H](c2ccc3c(NCc4ccccc4)nc(Cl)nc3c2)[C@H](O)[C@@H]1O ; OC[C@H]1O[C@@H](c2ccc3c(NCc4ccccc4)ncnc3c2)[C@H](O)[C@@H]1O ; O=P(Cl)(Cl)CP(=O)(Cl)Cl ; O=P(O)(O)CP(=O)(O)OC[C@H]1O[C@@H](c2ccc3c(NCc4ccccc4)ncnc3c2)[C@H](O)[C@@H]1O]

Step A: To a solution of (3R,4S,5R)-2-[4-(benzylamino)-2-chloroquinazolin-7-yl]-5-(hydroxymethyl)oxolane-3,4-diol (100 mg, 0.25 mmol) in MeOH (5 mL) was added 10% Pd/C (80 mg). The reaction was stirred at rt for 1.5 h under a H₂ atmosphere. The reaction was filtered and the filtrate was concentrated in vacuo to give (3R,4S,5R)-2-[4-(benzylamino)quinazolin-7-yl]-5-(hydroxymethyl)oxolane-3,4-diol (90 mg, 90% yield) as a yellow oil. Mass Spectrum (ESI) m/z=367.9 (M+1).

Step B: To a solution of (3R,4S,5R)-2-[4-(benzylamino)quinazolin-7-yl]-5-(hydroxymethyl)oxolane-3,4-diol (70 mg, 0.19 mmol) in trimethyl phosphate (1 mL) was added a cold solution of [(dichlorophosphoryl)methyl]phosphonoyl dichloride (237 mg, 0.95 mmol) in trimethyl phosphate (1.0 mL) dropwise at 0° C. The reaction was stirred for 4 h. TEAC (0.5 M, 0.38 mL) was added to the reaction carefully and the reaction was stirred at this temperature for 15 mins, then warmed to room temperature and continued to stir for 1 h. Trimethyl phosphate was extracted using tert-butyl methyl ether (5 mL×2) and the aqueous layer was basified with ammonium hydroxide to pH ˜7-8. Then the solution was purified by Prep-HPLC using a gradient of 0.2% formic acid in water/ACN from 90:10 to 75:25, and suitable fractions were pooled and lyophilized to give [({[(2R,3S,4R,5S)-5-[4-(benzylamino)quinazolin-7-yl]-3,4-dihydroxyoxolan-2-yl]methoxy}(hydroxy)phosphoryl)methyl]phosphonic acid (8.1 mg, 10% yield) as a white solid.

¹H NMR (400 MHz, DMSO-d6) δ 10.96-10.00 (m, 1H), 9.30-8.82 (m, 2H), 8.62-8.13 (m, 2H), 7.92-7.59 (m, 1H), 7.50-7.19 (m, 4H), 5.06-4.83 (m, 1H), 4.96-4.85 (m, 3H), 4.24-3.97 (m, 2H), 3.93-3.79 (m, 2H), 2.43-1.92 (m, 2H). Mass Spectrum (ESI) m/z=525.385 (M+1). Mass Spectrum (ESI) m/z=525.8 (M+1).

### Example S13

### Synthesis of (((((2R,3R,4S,5S)-5-(4-(benzylamino)-2-chloroquinazolin-7-yl)-4-fluoro-3-hydroxytetrahydrofuran-2-yl)methoxy)(hydroxy)phosphoryl)methyl)phosphonic acid

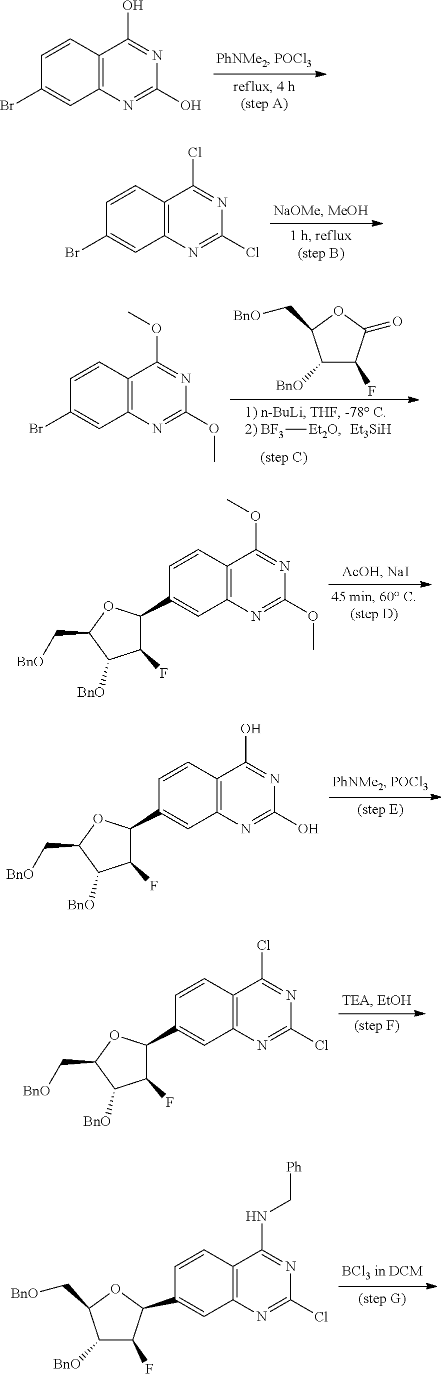

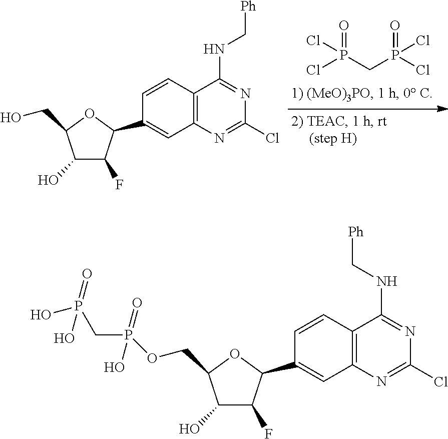 [SMILES: OC[C@H]1O[C@@H](c2ccc3c(NCc4ccccc4)nc(Cl)nc3c2)[C@@H](F)[C@@H]1O ; O=P(Cl)(Cl)CP(=O)(Cl)Cl ; O=P(O)(O)CP(=O)(O)OC[C@H]1O[C@@H](c2ccc3c(NCc4ccccc4)nc(Cl)nc3c2)[C@@H](F)[C@@H]1O]

Step A: To a suspension of 7-bromoquinazoline-2,4-diol (14 g, 58.35 mmol) in POCl₃ (140 ml) was added N,N-dimethylaniline (14 g, 116.69 mmol) slowly. The mixture was stirred at 100° C. for 4 hs. Then the reaction was concentrated and quenched with water, extracted with DCM (100 mL×2). The combined organic layers were washed with sat. NaHCO₃ solution and brine, dried over Na₂SO₄, filtered and the filtrate was concentrated and purified by CombiFlash® (EA/PE=0-10%) to give 7-bromo-2,4-dichloroquinazoline (13 g, 80% yield) as a white solid. Mass Spectrum (ESI) m/z=277.9 (M+1).

Step B: To a solution of 7-bromo-2,4-dichloroquinazoline (1 g, 3.62 mmol) in MeOH (30 mL) was added sodium methanolate (1.96 g, 36.2 mmol) at 0° C. Then the mixture was stirred at 80° C. for 2 hs. The reaction was poured into water, the solid was collected and washed with water, dried to give 7-bromo-2,4-dimethoxyquinazoline (670 mg, 57% yield) as a white solid. Mass Spectrum (ESI) m/z=323.1 (M+1).

Step C: To a solution of 7-bromo-2,4-dimethoxyquinazoline (400 mg, 1.49 mmol) in anhydrous THF (8 mL) stirring at −78° C., 0.81 mL of 2.4 M n-butyllithium solution in pentane (1.94 mmol) was carefully added dropwise. The reaction mixture was stirred for 30 min. at ˜78° C., then a solution of (3S,4R,5R)-4-(benzyloxy)-5-[(benzyloxy)methyl]-3-fluorooxolan-2-one (443 mg, 1.34 mmol) in 2 mL anhydrous THF was added dropwise. The reaction was stirred for 2.5 h at −78° C., then for 3 h at −30° C. After quenching with sat. aq. NH₄Cl solution in cold, the aqueous layer was extracted with Et₂O, dried over anhydrous Na₂SO₄, filtered, and the filtrate was evaporated under reduced pressure. The dried, crude product was dissolved in anhydrous CH₂Cl₂ (10 mL) and stirred at −78° C. To this mixture, 4 ml of triethylsilane (25 mmol) was added dropwise, followed by 2.9 g of boron trifluoride diethyl etherate (25 mmol). The reaction was stirred overnight at room temperature. After quenching with sat. aq. NaHCO₃ solution, the mixture was extracted with Et₂O. The organic layer was dried over anhydrous Na₂SO₄, filtered, the filtrate was concentrated and purified by CombiFlash® (12 g, EA/PE=0-21%) to give impure product, which was purified by prep-HPLC and chiral HPLC to give 7-((2S,3R,4R,5R)-4-(benzyloxy)-5-((benzyloxy)methyl)-3-fluorotetrahydrofuran-2-yl)-2,4-dimethoxyquinazoline (100 mg, 13.3% yield) as a white solid and 7-((2R,3R,4R,5R)-4-(benzyloxy)-5-((benzyloxy)methyl)-3-fluorotetrahydrofuran-2-yl)-2,4-dimethoxyquinazoline (200 mg, 26.7% yield) as a white solid. Mass Spectrum (ESI) m/z=504.5 (M+1).

Step D: To a solution of 7-((2S,3R,4R,5R)-4-(benzyloxy)-5-((benzyloxy)methyl)-3-fluorotetrahydrofuran-2-yl)-2,4-dimethoxyquinazoline (504 mg, 1 mmol) in glacial acetic acid (8 mL) was added sodium iodide (750 mg, 5 mmol). The reaction mixture was heated to 60-65° C. for 45 min, and then the volatiles were removed in vacuo. The residue was dissolved in EtOAc (50 mL) and washed with saturated Na₂SO₃ (aq) (30 mL×3) and saturated aq. sodium bicarbonate solution (20 mL×2). The aqueous layers were extracted with EtOAc (2×30 mL). The combined organics were dried over Na₂SO₄ and concentrated in vacuo. The residue was purified by silica gel column chromatography (MeOH/DCM=1:20) to give 7-((2S,3R,4R,5R)-4-(benzyloxy)-5-((benzyloxy)methyl)-3-fluorotetrahydrofuran-2-yl)quinazoline-2,4-diol (440 mg, 82% yield) as a colorless oil. Mass Spectrum (ESI) m/z=477.1 (M+1).

Step E: To a solution of 7-((2S,3R,4R,5R)-4-(benzyloxy)-5-((benzyloxy)methyl)-3-fluorotetrahydrofuran-2-yl)quinazoline-2,4-diol (440 mg, 0.92 mmol) in phosphorus oxychloride (7 mL) was added N,N-dimethylaniline (224 mg, 1.85 mmol). The reaction mixture was stirred under refluxing conditions for 2 h. And then was concentrated and purified by silica gel column chromatography (EA/PE=3:7) to give 7-((2S,3R,4R,5R)-4-(benzyloxy)-5-((benzyloxy)methyl)-3-fluorotetrahydrofuran-2-yl)-2,4-dichloroquinazoline (260 mg, 55% yield) as a colorless oil. Mass Spectrum (ESI) m/z=513.4 (M+1).

Step F: To a solution of 7-((2S,3R,4R,5R)-4-(benzyloxy)-5-((benzyloxy)methyl)-3-fluorotetrahydrofuran-2-yl)-2,4-dichloroquinazoline (260 mg, 0.51 mmol) in ethanol (5 mL) was added trimethylamine (103 mg, 1.02 mmol) and benzylamine (82 mg, 0.76 mmol). The mixture was stirred at 60° C. for 2 h. Then the reaction solution was concentrated and purified by CombiFlash® (4 g, EA/PE=0-60%) to give N-benzyl-7-((3R,4R,5R)-4-(benzyloxy)-5-((benzyloxy)methyl)-3-fluorotetrahydrofuran-2-yl)-2-chloroquinazolin-4-amine (200 mg, 61% yield) as a colorless oil. Mass Spectrum (ESI) m/z=584.1 (M+1).

Step G: To a solution of N-benzyl-7-[(3R,4R,5R)-4-(benzyloxy)-5-[(benzyloxy)methyl]-3-fluorooxolan-2-yl]-2-chloroquinazolin-4-amine (200 mg, 0.34 mmol) in DCM (5 mL) was added trichloroborane (1 M in DCM, 3.4 mL, 3.4 mmol) at −70° C. The mixture was stirred at −70° C. for 1 h. Then the reaction was brought to −30° C. over a period of 30 min, and quenched by adding a mixture of methanol/chloroform (2:1, 10 mL). After the reaction mixture reached rt, it was neutralized with NH₃ in methanol (10%, 10 mL) and concentrated. The residue was purified by CombiFlash® (5% NH₃ in MeOH/DCM=0-5%) to give (2R,3R,4S)-5-(4-(benzylamino)-2-chloroquinazolin-7-yl)-4-fluoro-2-(hydroxymethyl)tetrahydrofuran-3-ol (100 mg, 74% yield) as an off-white solid. Mass Spectrum (ESI) m/z=404 (M+1).

Step H: To a solution of (2R,3R,4S)-5-[4-(benzylamino)-2-chloroquinazolin-7-yl]-4-fluoro-2-(hydroxymethyl)oxolan-3-ol (100 mg, 0.25 mmol) in trimethyl phosphate (1.5 mL) at 0° C. was added a cold solution of methylenebis(phosphonic dichloride) (308 mg, 1.23 mmol) in trimethyl phosphate (0.5 mL) dropwise. Then the reaction solution was stirred at 0° C. for 3 h. TEAC (0.5 M, 6 mL) was added to the reaction carefully, and the reaction was stirred at this temperature for 15 mins, then warmed to room temperature and continued to stir for 1 h. Trimethyl phosphate was extracted using tert-butyl methyl ether (5 mL×2) and the aqueous layer was basified with ammonium hydroxide to pH ˜7-8. Then the solution was purified by Prep-HPLC using a gradient of 0.2% formic acid in water/ACN from 85:15 to 45:55 to give (((((2R,3R,4S)-5-(4-(benzylamino)-2-chloroquinazolin-7-yl)-4-fluoro-3-hydroxytetrahydrofuran-2-yl)methoxy)(hydroxy)phosphoryl)methyl)phosphonic acid (25 mg, 16% yield) as a white solid.

¹H NMR (400 MHz, DMSO-d6) δ ppm 9.37-9.29 (m, 1H), 8.31 (d, J=8.6 Hz, 1H), 7.66 (s, 1H), 7.60 (d, J=8.4 Hz, 1H), 7.45-7.25 (m, 5H), 5.35-5.27 (m, 1H), 5.18-5.05 (m, 1H), 4.76 (d, J=5.7 Hz, 2H), 4.30-4.26 (m, 1H), 4.14-4.02 (m, 3H), 2.22 (t, J=20.1 Hz, 2H). Mass Spectrum (ESI) m/z=561.9 (M+1).

### Example S14

### Synthesis of [({[(2R,3R,4S, 5R)-5-[4-(benzylamino)-2-chloroquinazolin-7-yl]-4-fluoro-3-hydroxyoxolan-2-yl]methoxy}(hydroxy)phosphoryl)methyl]phosphonic acid

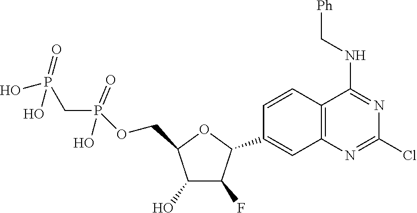 [SMILES: O=P(O)(O)CP(=O)(O)OC[C@H]1O[C@H](c2ccc3c(NCc4ccccc4)nc(Cl)nc3c2)[C@@H](F)[C@@H]1O]

[({[(2R,3R,4S)-5-[4-(benzylamino)-2-chloroquinazolin-7-yl]-4-fluoro-3-hydroxyoxolan-2-yl]methoxy}(hydroxy)phosphoryl)methyl]phosphonic acid was synthesized by procedures similar to the ones described in Example S13.

¹H NMR (400 MHz, DMSO-d6) δ 9.34 (t, J=5.8 Hz, 1H), 8.34 (d, J=8.6 Hz, 1H), 7.66-7.52 (m, 2H), 7.46-7.25 (m, 5H), 5.30 (dd, J=19.7, 3.6 Hz, 1H), 5.00 (d, J=53.5 Hz, 1H), 4.76 (d, J=5.6 Hz, 2H), 4.37 (d, J=18.9 Hz, 1H), 4.22 (s, 1H), 4.07 (s, 2H), 2.35-2.02 (m, 2H). Mass Spectrum (ESI) m/z=561.822 (M+1).

### Example S15

### Synthesis of (((((2R,3R,4S,5S)-5-(2-chloro-4-(cyclopentyl(methyl)amino)quinazolin-7-yl)-4-fluoro-3-hydroxytetrahydrofuran-2-yl)methoxy)(hydroxy)phosphoryl)methyl)phosphonic acid

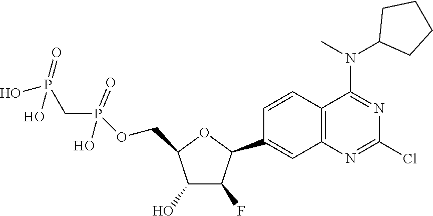 [SMILES: CN(c1nc(Cl)nc2cc([C@@H]3O[C@H](COP(=O)(O)CP(=O)(O)O)[C@@H](O)[C@@H]3F)ccc12)C1CCCC1]

(((((2R,3R,4S,5S)-5-(2-chloro-4-(cyclopentyl(methyl)amino)quinazolin-7-yl)-4-fluoro-3-hydroxytetrahydrofuran-2-yl)methoxy)(hydroxy)phosphoryl)methyl)phosphonic acid was prepared by procedures similar to the ones described in Example S13, replacing benzylamine with N-methlcyclopentanamine.

¹H NMR (400 MHz, DMSO-d6) δ ppm 8.19-8.08 (m, 1H), 7.67 (d, J=12.7 Hz, 1H), 7.56-7.54 (m, 1H), 5.34-5.27 (m, 1H), 5.18-5.06 (m, 1H), 4.90-4.87 (m, 1H), 4.27-4.22 (m, 1H), 4.11-4.08 (m, 3H), 3.19 (s, 3H), 2.40-2.24 (m,2H), 2.08-1.99 (m, 2H), 1.90-1.80 (m, 4H), 1.65-1.57 (m, 2H). (m, 2H), 2.32-2.16 (m, 2H). Mass Spectrum (ESI) m/z=553.8 (M+1).

### Example S16

### Synthesis of (((((2R,3S,4R,5R)-5-(4-(benzylamino)-2-chloroquinazolin-7-yl)-5-cyano-3,4-dihydroxytetrahydrofuran-2-yl)methoxy)(hydroxy)phosphoryl)methyl)phosphonic acid

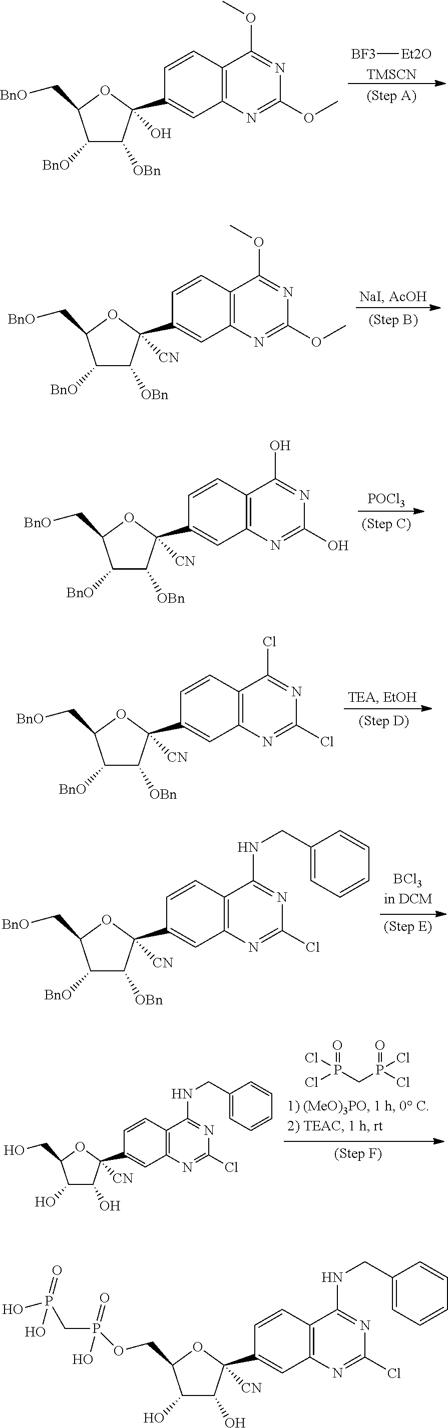

Step A: To a solution of (2S,3R,4R,5R)-3,4-bis(benzyloxy)-5-[(benzyloxy)methyl]-2-(2,4-dimethoxyquinazolin-7-yl)oxolan-2-ol (2 g, 3.29 mmol) in anhydrous CH₂Cl₂ (40 ml) at 0° C. under nitrogen was added BF₃—Et₂O (2 mL, 15.1 mmol). The mixture stirred for 10 min. TMSCN (1.9 g, 19.4 mmol) was added and the reaction mixture was stirred at 0° C. for 1 h. Then aqueous NaHCO₃ solution was added and the reaction mixture was extracted with EA. The organic layer was washed with water and brine, dried over NaSO₄, filtered and the filtrate was concentrated and purified by silica gel column chromatography (hexanes:EtOAc=4:1 to 1:1) to afford (2R,3R,4R,5R)-3,4-bis(benzyloxy)-5-[(benzyloxy)methyl]-2-(2,4-dimethoxyquinazolin-7-yl)oxolane-2-carbonitrile as a yellow oil (980 mg, 46.2% yield). Mass Spectrum (ESI) m/z=618.1 (M+1).

Step B: (2R,3R,4R,5R)-3,4-bis(benzyloxy)-5-[(benzyloxy)methyl]-2-(2,4-dimethoxyquinazolin-7-yl)oxolane-2-carbonitrile (920 mg, 1.49 mmol) was dissolved in AcOH (20 ml) and NaI (1.1 g, 7.45 mmol) was added. Then the mixture was stirred at 60° C. for 1 h, concentrated under reduced pressure, diluted with EA, washed with water and sat. Na₂S₂O₃ solution, dried over Na₂SO₄, filtered and the filtrate was concentrated and purified by silica gel column chromatography (hexanes:EtOAc 4:1 to 1:1) to afford (2R,3R,4R,5R)-3,4-bis(benzyloxy)-5-[(benzyloxy)methyl]-2-(2,4-dihydroxyquinazolin-7-yl)oxolane-2-carbonitrile as a yellow oil (630 mg, 69.1% yield). Mass Spectrum (ESI) m/z=590.1 (M+1).

Step C: To a solution of (2R,3R,4R,5R)-3,4-bis(benzyloxy)-5-[(benzyloxy)methyl]-2-(2,4-dihydroxyquinazolin-7-yl)oxolane-2-carbonitrile (630 mg, 1.07 mmol) in POCl₃ (9 ml) stirred under nitrogen, N,N-dimethylaniline(259 mg, 2.14 mmol) was added. Then the reaction mixture was stirred at 110° C. for 3 h. Then the reaction was concentrated, quenched with water and extracted with DCM (10 mL×2). The combined organic layers were washed with sat. NaHCO₃ solution and brine, dried over Na₂SO₄, filtered and the filtrate was concentrated and purified by, silica gel column chromatography (hexanes:EtOAc 4:1 to 1:1) to give (2R,3R,4S,5R)-4-(benzyloxy)-5-[(benzyloxy)methyl]-2-(2,4-dichloroquinazolin-7-yl)-3-hydroxyoxolane-2-carbonitrile as a yellow oil (480 mg, 79.4% yield). Mass Spectrum (ESI) m/z=626.0 (M+1).

Step D: To a solution of (2R,3R,4R,5R)-3,4-bis(benzyloxy)-5-[(benzyloxy)methyl]-2-(2,4-dichloroquinazolin-7-yl)oxolane-2-carbonitrile (480 mg, 0.77 mmol) in EtOH (10 ml) was added DIEA (198 mg, 1.54 mmol) and benzylamine (90.5 mg, 0.85 mmol). Then the reaction mixture was stirred at 70° C. for 1 h. The resulting solution was concentrated and the crude product was purified by silica gel column chromatography (hexanes:EtOAc 4:1 to 1:1) to give (2R,3R,4R,5R)-2-[4-(benzylamino)-2-chloroquinazolin-7-yl]-3,4-bis(benzyloxy)-5-[(benzyloxy)methyl]oxolane-2-carbonitrile as a yellow oil (410 mg, 72.9% yield). Mass Spectrum (ESI) m/z=697.1 (M+1).

Step E: To a solution of (2R,3R,4R,5R)-2-[4-(benzylamino)-2-chloroquinazolin-7-yl]-3,4-bis(benzyloxy)-5-[(benzyloxy)methyl]oxolane-2-carbonitrile (410 mg, 0.59 mmol) in DCM (2 ml) at −70° C. was added BCl₃ (1 M in DCM, 5.89 mL, 5.89 mmol) slowly. The reaction mixture was stirred at −70° C. for 1 h. Then the reaction was brought to −30° C. over a period of 30 min and quenched by adding a mixture of methanol and chloroform (2:1, 10 mL). After the reaction mixture reached rt, it was neutralized with NH₃ in methanol (10%, 10 mL) and concentrated. The residue was purified by silica gel column chromatography (DCM/MeOH=50:1 to 10:1) to give (2R,3R,4S,5R)-2-[4-(benzylamino)-2-chloroquinazolin-7-yl]-3,4-dihydroxy-5-(hydroxymethyl)oxolane-2-carbonitrile as a yellow oil (100 mg, 39.1% yield). Mass Spectrum (ESI) m/z=427.0 (M+1).

Step F: To a solution of (2R,3R,4S,5R)-2-[4-(benzylamino)-2-chloroquinazolin-7-yl]-3,4-dihydroxy-5-(hydroxymethyl)oxolane-2-carbonitrile (100 mg, 0.23 mmol) in (MeO)₃PO (1 ml) at 0° C. was added a cold solution of [(dichlorophosphoryl)methyl]phosphonoyl dichloride (294 mg, 1.17 mmol) in (MeO)₃PO (0.2 ml) slowly. The reaction mixture was stirred at 0° C. for 4 h. TEAC (0.5 M, 6 mL) was added to the reaction carefully and the reaction was stirred at this temperature for 15 mins, then warmed to rt and continued to stir for lh. Trimethyl phosphate was extracted using tert-butyl methyl ether (5 mL×2) and the aqueous layer was basified with ammonium hydroxide to pH ˜7-8. Then the solution was purified by Prep-HPLC using a gradient of 0.2% formic acid in water/ACN from 85:15 to 45:55 to give [{[(2R,3S,4R,5R)-5-[4-(benzylamino)-2-chloroquinazolin-7-yl]-5-cyano-3,4-dihydroxyoxolan-2-yl]methoxy}(hydroxy)phosphoryl)methyl]phosphonic acid as a white solid (12 mg, 8.5% yield).

¹H NMR (400 MHz, D₂O) δ 8.14 (d, J=7.6 Hz, 1H), 7.98 (s, 1H), 7.77 (d, J=7.7 Hz, 1H), 7.43-7.20 (m, 5H), 4.87-4.82 (m,2H), 4.52-4.46(m, 1H), 4.40-4.31 (m, 1H), 4.29-4.22 (m, 1H), 4.21-4.07 (m,2H), 2.2-2.04 (m, 2H). Mass Spectrum (ESI) m/z=584.6(M+1).

### Example S17

### Synthesis of (((((2R,3S,4R,5S)-5-(4-(benzylamino)-2-methylquinazolin-7-yl)-3,4-dihydroxytetrahydrofuran-2-yl) methoxy)(hydroxy)phosphoryl)methyl)phosphonic acid

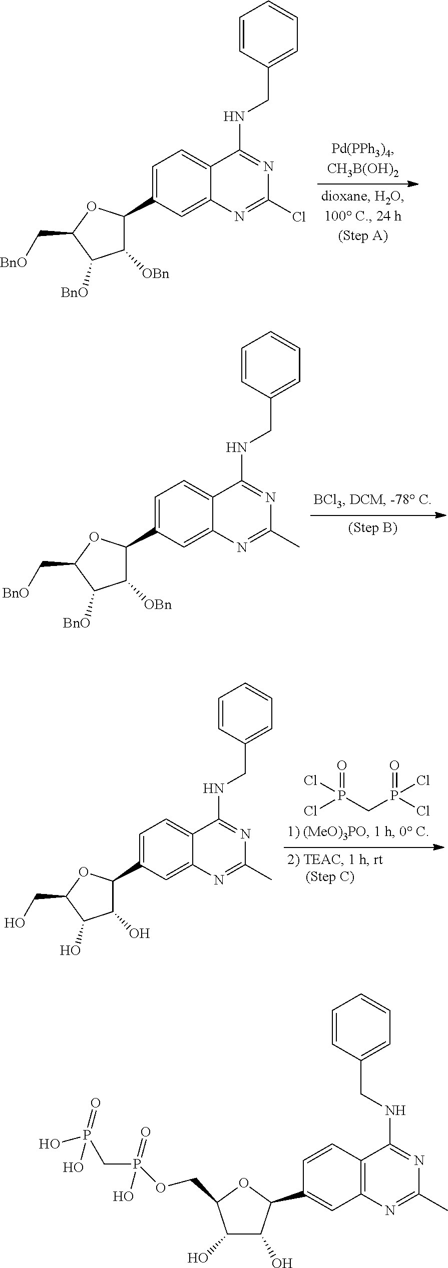 [SMILES: Clc1nc(NCc2ccccc2)c2ccc([C@@H]3O[C@H](COCc4ccccc4)[C@@H](OCc4ccccc4)[C@H]3OCc3ccccc3)cc2n1 ; Cc1nc(NCc2ccccc2)c2ccc([C@@H]3O[C@H](COCc4ccccc4)[C@@H](OCc4ccccc4)[C@H]3OCc3ccccc3)cc2n1 ; Cc1nc(NCc2ccccc2)c2ccc([C@@H]3O[C@H](CO)[C@@H](O)[C@H]3O)cc2n1 ; O=P(Cl)(Cl)CP(=O)(Cl)Cl ; Cc1nc(NCc2ccccc2)c2ccc([C@@H]3O[C@H](COP(=O)(O)CP(=O)(O)O)[C@@H](O)[C@H]3O)cc2n1]

Step A: A mixture of N-benzyl-7-[(2S,3S,4R,5R)-3,4-bis(benzyloxy)-5-[(benzyloxy)methyl]oxolan-2-yl]-2-chloroquinazolin-4-amine (1.0 g, 1.56 mmol), Pd(PPh₃)₄ (90 mg, 0.08 mmol), K₂CO₃ (432 mg, 3.12 mmol) and CH₃B(OH)₂ (188 mg, 3.12 mmol) in dioxane (9 mL) and water (1 mL) was stirred at 120° C. for 3 h under a N₂ atmosphere. The solvent was removed under reduced pressure and the residue was purified by CombiFlash® (4 g, eluting with PE:EA=5:1) to give N-benzyl-7-[(3S,4R,5R)-3,4-bis(benzyloxy)-5-[(benzyloxy)methyl]oxolan-2-yl]-2-chloroquinazolin-4-amine (330 mg, 29% yield) as a yellow oil. Mass Spectrum (ESI) m/z=652.2 (M+1).

Step B: To a solution of N-benzyl-7-[(3S,4R,5R)-3,4-bis(benzyloxy)-5-[(benzyloxy)methyl]oxolan-2-yl]-2-chloroquinazolin-4-amine (330 mg, 0.51 mmol) in DCM (10 mL) was added BCl₃ (1 M in DCM, 4.8 mL, 4.8 mmol) dropwise at −78° C. under a N₂ atmosphere. The reaction solution was stirred at −78° C. for 1 h. Then the reaction was brought to −30° C. over a period of 30 min and quenched by adding a mixture of methanol and chloroform (2:1, 10 mL). After the reaction mixture reached to rt, it was neutralized with NH₃ in methanol (10%, 10 mL) and concentrated. The residue was purified by CombiFlash® (4 g, eluting with DCM:MeOH:NH₄OH=70:30:5) to give (2S,3R,4S,5R)-2-[4-(benzylamino)-2-methylquinazolin-7-yl]-5-(hydroxymethyl)oxolane-3,4-diol (80 mg, 41% yield). Mass Spectrum (ESI) m/z=382.1 (M+1).

Step C: To a solution of (2S,3R,4S,5R)-2-[4-(benzylamino)-2-methylquinazolin-7-yl]-5-(hydroxymethyl)oxolane-3,4-diol (80 mg, 0.20 mmol) in trimethyl phosphate (1 mL) at 0° C. was added a cold solution of methylenebis(phosphonic dichloride) (261 mg, 1.04 mmol) in trimethyl phosphate (1 mL) dropwise. Then the reaction solution was stirred at 0° C. for 1 h. TEAC (0.5 M, 1.4 mL) was added to the reaction carefully and the reaction was stirred at this temperature for 15 mins, then warmed to room temperature and continued to stir for lh. Trimethyl phosphate was extracted using tert-butyl methyl ether (5 mL×2) and the aqueous layer was basified with ammonium hydroxide to pH ˜7-8. Then the solution was purified by Prep-HPLC using a gradient of 0.2% formic acid in water/ACN from 90:10 to 70:30, and suitable fractions were pooled and lyophilized to give the final product. (4 mg, 3.5% yield).

¹H NMR (400 MHz, D₂O) δ 8.04 (d, J=8.6 Hz, 1H), 7.91 (s, 1H), 7.58 (d, J=8.7 Hz, 1H), 7.37-7.22 (m, 5H), 4.94-4.90 (m, 1H), 4.91 (d, J=6.7 Hz, 1H), 4.88 (s, 2H), 4.28-4.20 (m, 2H), 4.15-4.03 (m, 3H), 2.55 (s, 3H), 2.19 (t, J=18.5 Hz, 2H). Mass Spectrum (ESI) m/z=540.0 (M+1).

### Example S18

### Synthesis of [({[(2R,3S,4R,5S)-5-[4-(benzylamino)-2-ethenylquinazolin-7-yl]-3,4-dihydroxyoxolan-2-yl]methoxy}(hydroxy)phosphoryl)methyl]phosphonic acid

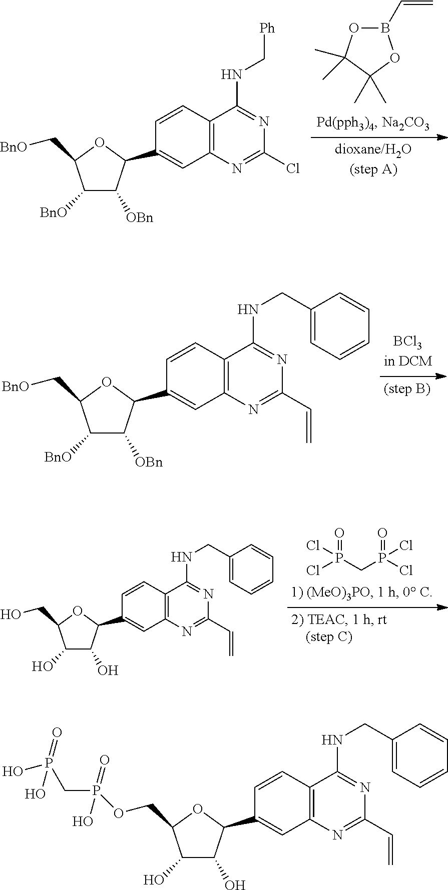 [SMILES: Clc1nc(NCc2ccccc2)c2ccc([C@@H]3O[C@H](COCc4ccccc4)[C@@H](OCc4ccccc4)[C@H]3OCc3ccccc3)cc2n1 ; C=CB1OC(C)(C)C(C)(C)O1 ; C=Cc1nc(NCc2ccccc2)c2ccc([C@@H]3O[C@H](COCc4ccccc4)[C@@H](OCc4ccccc4)[C@H]3OCc3ccccc3)cc2n1 ; C=Cc1nc(NCc2ccccc2)c2ccc([C@@H]3O[C@H](CO)[C@@H](O)[C@H]3O)cc2n1 ; O=P(Cl)(Cl)CP(=O)(Cl)Cl ; C=Cc1nc(NCc2ccccc2)c2ccc([C@@H]3O[C@H](COP(=O)(O)CP(=O)(O)O)[C@@H](O)[C@H]3O)cc2n1]

Step A: To a solution of N-benzyl-2-chloro-7-[(2R,3R,4S,5R)-5-ethyl-3,4-dimethyloxolan-2-yl]quinazolin-4-amine (600 mg, 0.89 mmol) in dioxane (12 mL) was added tributyl(vinyl)tin (340 mg, 1.07 mmol) and bis(triphenylphosphine)palladium(II) chloride (125 mg, 0.18 mmol). The reaction was stirred at 100° C. for 16 h under a N₂ atmosphere. Then allowed to cool to room temperature. The mixture was concentrated and purified by silica gel column chromatography (hexanes: ethyl acetate 80:20) to give N-benzyl-7-[(2S,3 S,4R,5R)-3,4-bis(benzyloxy)-5-[(benzyloxy)methyl]oxolan-2-yl]-2-ethenylquinazolin-4-amine (310 mg, 47% yield) as a yellow oil. Mass Spectrum (ESI) m/z=663.8 (M+1).

Step B: To a solution of N-benzyl-7-[(2S,3S,4R,5R)-3,4-bis(benzyloxy)-5-[(benzyloxy)methyl]oxolan-2-yl]-2-ethenylquinazolin-4-amine (310 mg, 0.47 mmol) in DCM (15 mL) was added boron trichloride (1 M in DCM, 4.7 mL, 4.7 mmol) at −78° C. The reaction was stirred at −78° C. for 2 h. Then the reaction was brought to −30° C. over a period of 30 min and quenched by adding a mixture of methanol and chloroform (2:1, 10 mL). After the reaction mixture had warmed to rt, it was neutralized with NH₃ in methanol (10%, 10 mL) and concentrated. The residue was purified by CombiFlash® (eluting with MeOH/DCM=5:95) to give (2S,3R,4S,5R)-2-[4-(benzylamino)-2-ethenylquinazolin-7-yl]-5-(hydroxymethyl)oxolane-3,4-diol (160 mg, 87% yield) as a yellow solid. Mass Spectrum (ESI) m/z=393.8(M+1).

Step C: To a solution of (2S,3R,4S,5R)-2-[4-(benzylamino)-2-ethenylquinazolin-7-yl]-5-(hydroxymethyl)oxolane-3,4-diol (100 mg, 0.25 mmol) in trimethyl phosphate (1.5 mL) was added a cold solution of [(dichlorophosphoryl)methyl]phosphonoyl dichloride (312 mg, 1.25 mmol) in trimethyl phosphate (1 mL) dropwise at 0° C. The reaction was stirred for 4 h. TEAC (0.5 M, 1.75 mL) was added to the reaction carefully, and the reaction was stirred at this temperature for 15 mins, then warmed to room temperature and continued to stir for 1 h. Trimethyl phosphate was extracted using tert-butyl methyl ether (5 mL×2) and the aqueous layer was basified with ammonium hydroxide to pH ˜7-8. Then the solution was purified by Prep-HPLC using a gradient of 0.2% formic acid in water/ACN from 90:10 to 75:25, and suitable fractions were pooled and lyophilized to give [({[(2R,3S,4R,5S)-5-[4-(benzylamino)-2-ethenylquinazolin-7-yl]-3,4-dihydroxyoxolan-2-yl]methoxy}(hydroxy)phosphoryl)methyl]phosphonic acid (13 mg, 90% yield) as a white solid.

¹H NMR (400 MHz, D₂O) δ 8.03-7.90 (m, 2H), 7.21 (s, 1H), 7.37-7.21 (m, 5H), 6.80-6.61 (m, 2H), 6.04-5.87 (m, 1H), 4.86-4.68 (m, 3H), 4.24-4.20 (m, 2H), 4.06-4.03 (m, 2H), 3.98-3.86 (m, 1H), 2.17-2.03 (m, 2H). Mass Spectrum (ESI) m/z=552.1 (M+1).

### Example S19

### Synthesis of (((((2R,3S,4R,5S)-5-(4-(benzylamino)-2-chloro-6-fluoroquinazolin-7-yl)-3,4-dihydroxytetrahydrofuran-2-yl)methoxy)(hydroxy)phosphoryl)methyl)phosphonic acid

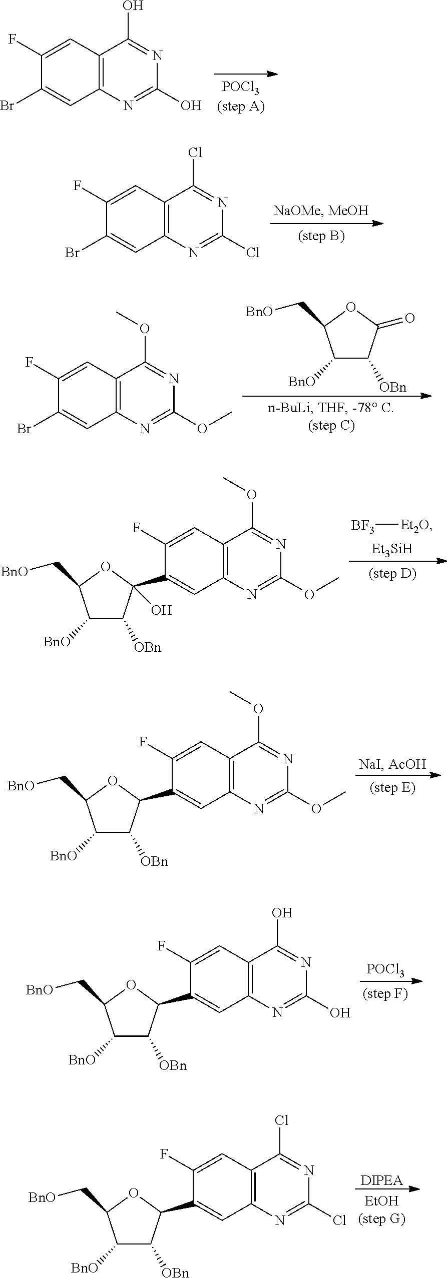

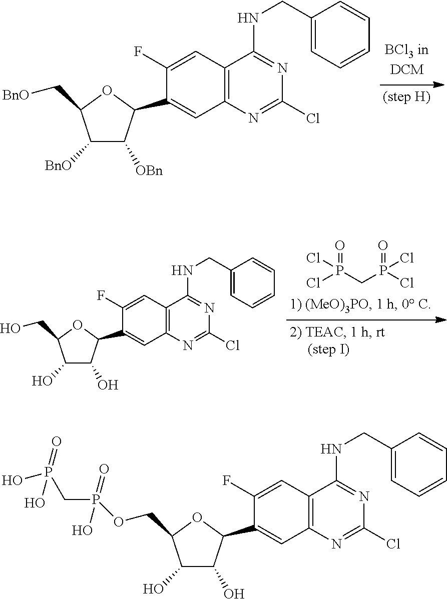 [SMILES: Fc1cc2c(NCc3ccccc3)nc(Cl)nc2cc1[C@@H]1O[C@H](COCc2ccccc2)[C@@H](OCc2ccccc2)[C@H]1OCc1ccccc1 ; OC[C@H]1O[C@@H](c2cc3nc(Cl)nc(NCc4ccccc4)c3cc2F)[C@H](O)[C@@H]1O ; O=P(Cl)(Cl)CP(=O)(Cl)Cl ; O=P(O)(O)CP(=O)(O)OC[C@H]1O[C@@H](c2cc3nc(Cl)nc(NCc4ccccc4)c3cc2F)[C@H](O)[C@@H]1O]

Step A: To a solution of 7-bromo-6-fluoroquinazoline-2,4-diol (900 mg, 3.47 mmol) in POCl₃ (10 mL) was added N,N-dimethylaniline (337 mg, 2.78 mmol). The mixture was stirred at 110° C. for 3 h. The reaction was concentrated, and the residue was dissolved in DCM (20 mL), poured into ice water, extracted with DCM (20 mL×2). The combined organic layers were washed with brine, dried, filtered and the filtrate was concentrated and purified by Combi- Flash (12 g, EA/PE=0-10%) to give 7-bromo-2,4-dichloro-6-fluoroquinazoline (750 mg, 69% yield) as a yellow solid. 1H NMR (400 MHz, CDCl₃) δ ppm 8.33 (d, J=6.2 Hz, 1H), 7.95 (d, J=7.8 Hz, 1H). Mass Spectrum (ESI) m/z=295.2 (M+23).

Step B: To a solution of 7-bromo-2,4-dichloro-6-fluoroquinazoline (750 mg, 2.53 mmol) in MeOH (10 mL) was added sodium methoxide (684 mg, 12.67 mmol). The mixture was stirred at 60° C. for 1 h. The mixture was concentrated and purified by CombiFlash® (12 g, EA/PE=0-10%) to give 7-bromo-6-fluoro-2,4-dimethoxyquinazoline (530 mg, 73% yield) as a white solid. 41 NMR (400 MHz, CDCl₃) δ ppm 8.07 (d, J=6.2 Hz, 1H), 7.75 (d, J=8.1 Hz, 1H), 4.21 (s, 3H), 4.11 (s, 3H).

Step C: To a solution of 7-bromo-6-fluoro-2,4-dimethoxyquinazoline (530 mg, 1.85 mmol) in THF (5 mL) was added n-butyllithium (2.4 M, 1.16 mL, 2.78 mmol) at −78° C. under a nitrogen atmosphere, followed by a solution of (3R,4R,5R)-3,4-bis(benzyloxy)-5-[(benzyloxy)methyl]oxolan-2-one (774 mg, 1.85 mmol) in THF (5 mL). The mixture was stirred at −78° C. for 1 h, then warmed to −30° C. The reaction was quenched with saturated aq. NH₄Cl solution and extracted with EA (10 mL×2). The combined organic layers were washed with brine, dried, filtered and the filtrate was concentrated and purified by CombiFlash® (4 g, EA/PE=0-20%) to give (3R,4R,5R)-3,4-bis(benzyloxy)-5-((benzyloxy)methyl)-2-(6-fluoro-2,4-dimethoxyquinazolin-7-yl)tetrahydrofuran-2-ol (650 mg, 53% yield) as a colorless oil. Mass Spectrum (ESI) m/z=627.1 (M+1).

Step D: To a solution of (3R,4R,5R)-3,4-bis(benzyloxy)-5-[(benzyloxy)methyl]-2-(6-fluoro-2,4-dimethoxy-4a,8a-dihydroquinazolin-7-yl)oxolan-2-ol (650 mg, 1.04 mmol) in DCM (10 mL) was added triethylsilane (1.2 g, 10.35 mmol) and boron trifluoride diethyl etherate (1.47 g, 10.35 mmol) at −78° C. The mixture was warmed to rt and stirred for lh. Then the reaction was quenched with saturated aq. NaHCO₃ solution and extracted with DCM (20 mL×2). The combined organic layers were washed with brine, dried, filtered and the filtrate was concentrated and purified by CombiFlash® (4 g, gradient elution, EA/PE=0:100-15:85) to give 7-[(3S,4R,5R)-3,4-bis(benzyloxy)-5-[(benzyloxy)methyl]oxolan-2-yl]-6-fluoro-2,4-dimethoxyquinazoline (450 mg, 67% yield) as a colorless oil. Mass Spectrum (ESI) m/z=611.1 (M+1).

Step E: To a solution of 7-[(3S,4R,5R)-3,4-bis(benzyloxy)-5-[(benzyloxy)methyl]oxolan-2-yl]-6-fluoro-2,4-dimethoxyquinazoline (480 mg, 0.79 mmol) in acetic acid (5 mL) was added sodium iodide (589 mg, 3.93 mmol). The mixture was stirred at 60° C. for 45 min. Then the solvent was removed under reduced pressure. The residue was dissolved in DCM and the organic layer was washed with sat. Na₂S₂O₄ solution and sat. NaHCO₃ solution. The combined aqueous layers were extracted with DCM. The combined organic layers were concentrated and purified by CombiFlash® (4 g, PE/EA=0-30%) to give 7-((3S,4R,5R)-3,4-bis(benzyloxy)-5-((benzyloxy)methyl)tetrahydrofuran-2-yl)-6-fluoroquinazoline-2,4-diol (400 mg, 82% yield) as a white solid. Mass Spectrum (ESI) m/z=583.1 (M+1).

Step F: To a suspension of 7-[(3S,4R,5R)-3,4-bis(benzyloxy)-5-[(benzyloxy)methyl]oxolan-2-yl]-6-fluoroquinazoline-2,4-diol (400 mg, 0.69 mmol) in POCl₃ (5 mL) was added N,N-dimethylaniline (67 mg, 0.55 mmol). The mixture was stirred at 110° C. for 2 h. Then the solvent was removed under reduced pressure and the residue was dissolved with DCM (10 mL). The solution was poured into ice-water. The organic layer was washed with brine, dried, filtered and the filtrate was concentrated and purified by CombiFlash® (4 g, EA/PE=0-10%) to give 7-((3S,4R,5R)-3,4-bis(benzyloxy)-5-((benzyloxy)methyl)tetrahydrofuran-2-yl)-2,4-dichloro-6-fluoroquinazoline (350 mg, 78% yield) as a yellow oil. Mass Spectrum (ESI) m/z=619.1 (M+1).

Step G: To 7-[(3S,4R,5R)-3,4-bis(benzyloxy)-5-[(benzyloxy)methyl]oxolan-2-yl]-2,4-dichloro-6-fluoroquinazoline (360 g, 0.58 mmol) in EtOH (5 mL) was added ethyldiisopropylamine (150 mg, 1.16 mmol), followed by benzylamine (75 mg, 0.7 mmol). The mixture was stirred at 70° C. for 1 h. The reaction mixture was concentrated and purified by CombiFlash® (4 g, EA/PE=0-30%) to give N-benzyl-7-((3S,4R,5R)-3,4-bis(benzyloxy)-5-((benzyloxy)methyl)tetrahydrofuran-2-yl)-2-chloro-6-fluoroquinazolin-4-amine (300 mg, 59% yield) as a yellow oil. Mass Spectrum (ESI) m/z=690.1(M+1).

Step H: To a solution of N-benzyl-7-[(3S,4R,5R)-3,4-bis(benzyloxy)-5-[(benzyloxy)methyl]oxolan-2-yl]-2-chloro-6-fluoroquinazolin-4-amine (300 mg, 0.44 mmol) in DCM (5 mL) was added trichloroborane (1M in DCM, 4.4 mL, 4.4 mmol) at −70° C. under a nitrogen atmosphere. The mixture was stirred at −70° C. for 1 h. Then the reaction was brought to −30° C. over a period of 30 min, and quenched by adding a mixture of methanol:chloroform (2:1, 10 mL). After the reaction mixture reached rt, it was neutralized with NH₃ in methanol (10%, 10 mL) and concentrated. The residue was purified by CombiFlash® (4 g, MeOH/DCM=0-20%) to give (3R,4S,5R)-2-(4-(benzylamino)-2-chloro-6-fluoroquinazolin-7-yl)-5-(hydroxymethyl)tetrahydrofuran-3,4-diol (100 mg, 54.7% yield) as a white solid. Mass Spectrum (ESI) m/z=420.0 (M+1).

Step I: To a solution of (3R,4S,5R)-2-[4-(benzylamino)-2-chloro-6-fluoroquinazolin-7-yl]-5-(hydroxymethyl)oxolane-3,4-diol (90 mg, 0.22 mmol) in trimethyl phosphate (1.2 mL) at 0° C. was added a cold solution of methylenebis(phosphonic dichloride) (269 mg, 1.08 mmol) in trimethyl phosphate (0.5 mL) dropwise. Then the reaction solution was stirred at 0° C. for 3 h. TEAC (0.5 M, 8 mL) was added to the reaction carefully and the reaction was stirred at this temperature for 15 mins, then warmed to room temperature and continued to stir for 1 h. Trimethyl phosphate was extracted using tert-butyl methyl ether (5 mL×2) and the aqueous layer was basified with ammonium hydroxide to pH 7-8. Then the solution was purified by Prep-HPLC using a gradient of 0.2% formic acid in water/ACN from 90:10 to 60:40 to give (((((2R,3S,4R)-5-(4-(benzylamino)-2-chloro-6-fluoroquinazolin-7-yl)-3,4-dihydroxytetrahydrofuran-2-yl)methoxy)(hydroxy)phosphoryl)methyl)phosphonic acid (15 mg, 14%) as a white solid.

¹H NMR (400 MHz, DMSO-d6) δ ppm 9.24 (t, J=5.7 Hz, 1H), 8.19 (d, J=11.1 Hz, 1H), 7.78 (d, J=6.7 Hz, 1H), 7.41-7.33 (m, 4H), 7.29-7.26 (m, 1H), 5.01 (d, J=4.3 Hz, 1H), 4.76 (d, J=5.7 Hz, 2H), 4.25-4.09 (m, 2H), 4.07-4.02 (m, 1H), 3.98-3.87 (m, 2H), 2.35-2.17 (m, 2H). Mass Spectrum (ESI) m/z=577.6 (M+1).

### Example S20

Synthesis of [({[(2R,3S,4R,5S)-5-[4-(benzylamino)-2-ethylquinazolin-7-yl]-3,4-dihydroxyoxolan-2-yl]methoxy}(hydroxy)phosphoryl)methyl]phosphonic acid

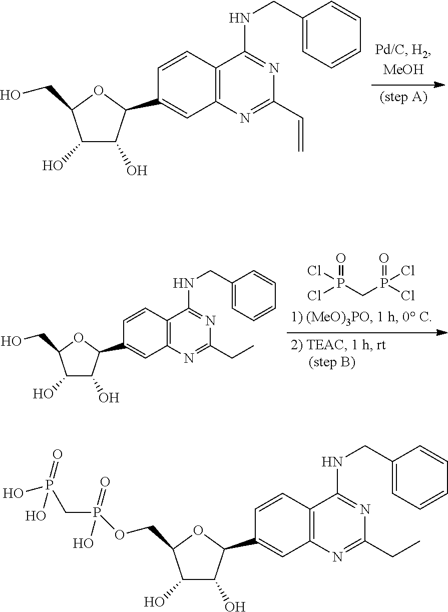 [SMILES: C=Cc1nc(NCc2ccccc2)c2ccc([C@@H]3O[C@H](CO)[C@@H](O)[C@H]3O)cc2n1 ; CCc1nc(NCc2ccccc2)c2ccc([C@@H]3O[C@H](CO)[C@@H](O)[C@H]3O)cc2n1 ; O=P(Cl)(Cl)CP(=O)(Cl)Cl ; CCc1nc(NCc2ccccc2)c2ccc([C@@H]3O[C@H](COP(=O)(O)CP(=O)(O)O)[C@@H](O)[C@H]3O)cc2n1]

Step A: To a solution of (2S,3R,4S,5R)-2-[4-(benzylamino)-2-ethenylquinazolin-7-yl]-5-(hydroxymethyl)oxolane-3,4-diol (50 mg, 0.13 mmol) in MeOH (5 mL) was added 10% Pd/C (40 mg). The reaction was stirred at rt for 1.5 h under a H₂ atmosphere. The reaction was filtered and the filtrate was concentrated in vacuo to give (2S,3R,4S,5R)-2-[4-(benzylamino)-2-ethylquinazolin-7-yl]-5-(hydroxymethyl)oxolane-3,4-diol (44 mg, 80% yield) as a white solid. Mass Spectrum (ESI) m/z=396.1(M+1).

Step B: To a solution of (2S,3R,4S,5R)-2-[4-(benzylamino)-2-ethylquinazolin-7-yl]-5-(hydroxymethyl)oxolane-3,4-diol (40 mg, 0.1 mmol) in trimethyl phosphate (0.8 mL) was added a cold solution of [(dichlorophosphoryl)methyl]phosphonoyl dichloride (124 mg, 0.5 mmol) in trimethyl phosphate (0.7 mL) dropwise at 0° C. The reaction was stirred for 4 h. TEAC (0.5 M, 0.7 mL) was added to the reaction carefully and the reaction was stirred at this temperature for 15 mins, then warmed to room temperature and continued to stir for 1 h. Trimethyl phosphate was extracted using tert-butyl methyl ether (5 mL×2) and the aqueous layer was basified with ammonium hydroxide to pH ˜7-8. Then the solution was purified by Prep-HPLC using a gradient of 0.2% formic acid in water/ACN from 100:0 to 70:30, and suitable fractions were pooled and lyophilized to give [({[(2R,3S,4R,5S)-5-[4-(benzylamino)-2-ethylquinazolin-7-yl]-3,4-dihydroxyoxolan-2-yl]methoxy}(hydroxy)phosphoryl)methyl]phosphonic acid (6 mg, 10% yield) as a white solid.

¹H NMR (400 MHz, D₂O) δ 8.03 (d, J=8.6 Hz, 1H), 7.93 (s, 1H), 7.57 (d, J=8.8 Hz, 1H), 7.38-7.33 (m,2H), 7.31-7.26 (m,2H), 7.25-7.20 (m,1H), 4.94-4.86 (m, 3H), 4.26-4.20 (m, 2H), 4.13-4.03 (m, 3H), 2.81 (dd, J=15.0, 7.4 Hz, 2H), 2.13 (t, J=20.2 Hz, 2H), 1.23 (t, J=7.6 Hz, 3H). Mass Spectrum (ESI) m/z=553.6 (M+1).

### Example S21

### Synthesis of [({[(2R,3S,4R,5S)-5-[2-chloro-4-[(oxolan-3-ylmethyl)amino]quinazolin-7-yl]-3,4-dihydroxyoxolan-2-yl]methoxy}(hydroxy)phosphoryl)methyl]phosphonic acid

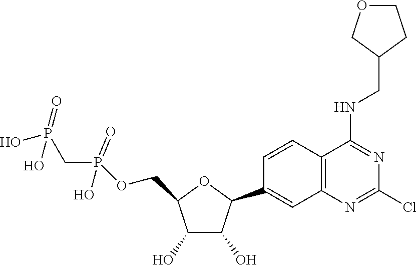 [SMILES: O=P(O)(O)CP(=O)(O)OC[C@H]1O[C@@H](c2ccc3c(NCC4CCOC4)nc(Cl)nc3c2)[C@H](O)[C@@H]1O]

[({[(2R,3S,4R,5S)-5-{2-chloro-4-[(oxolan-3-ylmethyl)amino]quinazolin-7-yl}-3,4-dihydroxyoxolan-2-yl]methoxy}(hydroxy)phosphoryl)methyl]phosphonic acid was prepared by procedures similar to the one described in Example S1, replacing benzylamine in Step G with (tetrahydrofuran-3-yl)methanamine.

¹H NMR (400 MHz, D₂O) δ 8.03 (d, J=8.6 Hz, 1H), 7.70 (m, 1H), 7.54 (s, 1H), 4.91-4.89 (m, 1H), 4.39-4.32 (m, 1H), 4.23 (dd, J=7.6, 3.8 Hz, 1H), 4.18-4.08 (m, 3H), 3.91-3.82 (m, 2H), 3.77-3.71 (m, 1H), 3.62-3.57 (m, 1H), 3.56-3.53 (m, 2H), 2.74 (m, 1H), 2.10-1.96 (m, 3H), 1.75-1.67 (m, 1H). Mass Spectrum (ESI) m/z=552.0 (M−1).

### Example S22

### Synthesis of (((((2R,3S,4R,5S)-5-(2-chloro-4-((tetrahydrofuran-3-yl)amino)quinazolin-7-yl)-3,4-dihydroxytetrahydrofuran-2-yl)methoxy)(hydroxy)phosphoryl)methyl)phosphonic acid

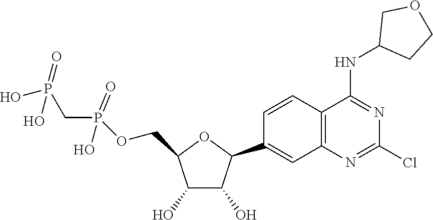 [SMILES: O=P(O)(O)CP(=O)(O)OC[C@H]1O[C@@H](c2ccc3c(NC4CCOC4)nc(Cl)nc3c2)[C@H](O)[C@@H]1O]

(((((2R,3S,4R,5S)-5-(2-chloro-4-((tetrahydrofuran-3-yl)amino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with tetrahydrofuran-3-amine.

¹H NMR (400 MHz, D₂O) δ 8.07 (d, J=8.6 Hz, 1H), 7.68 (dd, J=8.7, 1.4 Hz, 1H), 7.54 (s, 1H), 4.89 (d, J=6.8 Hz, 1H), 4.81-4.74 (m, 1H), 4.38-4.31 (m, 1H), 4.22 (d, J=3.9 Hz, 1H), 4.18-4.06 (m, 3H), 4.05-3.96 (m, 2H), 3.88-3.82 (m, 2H), 2.39-2.34 (m, 1H), 2.16-1.89 (m, 3H). Mass Spectrum (ESI) m/z=540.0 (M+1).

### Example S23

Synthesis of (((((2R,3S,4R,5S)-5-(2-chloro-4((2-chlorobenzyl)amino)quinazolin-7-yl)-3,4-dihydroxytetrahydrofuran-2-yl)methoxy)(hydroxy)phosphoryl)methyl)phosphonic acid

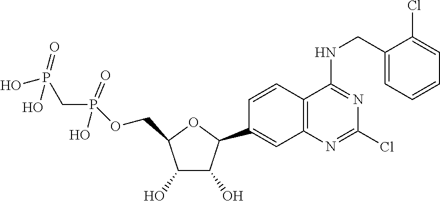 [SMILES: O=P(O)(O)CP(=O)(O)OC[C@H]1O[C@@H](c2ccc3c(NCc4ccccc4Cl)nc(Cl)nc3c2)[C@H](O)[C@@H]1O]

(((((2R,3S,4R,5S)-5-(2-chloro-4-((2-chlorobenzyl) amino) quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with (2-chlorophenyl)methanamine.

¹H NMR (400 MHz, D₂O) δ 8.08 (d, J=8.6 Hz, 1H), 7.75-7.69 (m, 1H), 7.56 (s, 1H), 7.42 (dd, J=7.6, 1.7 Hz, 1H), 7.36 (dd, J=7.2, 2.1 Hz, 1H), 7.29-7.20 (m, 2H), 4.90 (d, J=6.8 Hz, 1H), 4.81 (s, 2H), 4.37-4.32 (m, 1H), 4.22 (d, J=3.9 Hz, 1H), 4.19-4.06 (m, 3H), 2.00 (t, J=19.6 Hz, 2H). Mass Spectrum (ESI) m/z=593.7(M+1).

### Example S24

### Synthesis of [({[(2R,3S,4R,5S)-5-(2-chloro-4-{hexahydro-1H-cyclopenta[c]pyrrol-2-yl}quinazolin-7-yl)-3,4-dihydroxyoxolan-2-yl]methoxy}(hydroxy)phosphoryl)methyl]phosphonic acid

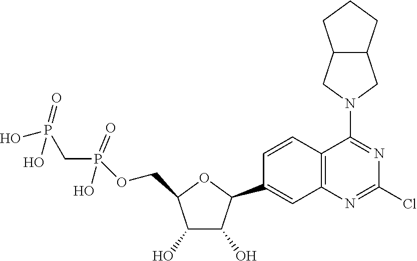 [SMILES: O=P(O)(O)CP(=O)(O)OC[C@H]1O[C@@H](c2ccc3c(N4CC5CCCC5C4)nc(Cl)nc3c2)[C@H](O)[C@@H]1O]

[({[(2R,3S,4R,5S)-5-(2-chloro-4- {hexahydro-1H-cyclopenta[c]pyrrol-2-yl}quinazolin-7-yl)-3,4-dihydroxyoxolan-2-yl]methoxy}(hydroxy)phosphoryl)methyl]phosphonic acid was synthesized by procedures similar to the ones described in Example S1, replacing benzylamine in Step G with octahydrocyclopenta[c]pyrrole.

¹H NMR (400 MHz, D₂O) δ 8.21 (d, J=8.8 Hz, 1H), 7.52 (d, J=8.8 Hz, 1H), 7.46 (s, 1H), 4.86 (d, J=7.2 Hz, 1H), 4.33-4.19 (m, 2H), 4.18-4.05 (m, 3H), 3.98 (s, 2H), 3.67 (d, J=12.5 Hz, 2H), 2.75 (s, 2H), 2.13 (t, J=19.6 Hz, 2H), 1.90-1.67 (m, 3H), 1.65-1.51 (m, 1H), 1.50-1.46 (m,2H). Mass Spectrum (ESI) m/z=561.7 (M−1).

### Example S25

### Synthesis of [({[(2R,3S,4R,5S)-5-[2-chloro-4-(2,3-dihydro-1H-inden-1-ylamino)quinazolin-7-yl]-3,4-dihydroxyoxolan-2-yl]methoxy}(hydroxy)phosphoryl)methyl]phosphonic acid

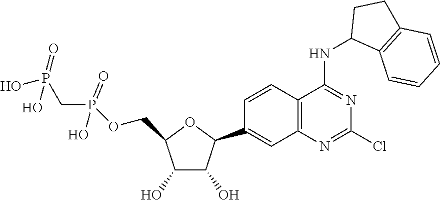 [SMILES: O=P(O)(O)CP(=O)(O)OC[C@H]1O[C@@H](c2ccc3c(NC4CCc5ccccc54)nc(Cl)nc3c2)[C@H](O)[C@@H]1O]

[({[(2R,3S,4R,5S)-5-[2-chloro-4-(2,3-dihydro-1H-inden-1-ylamino)quinazolin-7-yl]-3,4-dihydroxyoxolan-2-yl]methoxy}(hydroxy)phosphoryl)methyl]phosphonic acid was synthesized by procedures similar to the ones described in Example S1, replacing benzylamine in Step G with 2,3-dihydro-1H-inden-1-amine.

¹H NMR (400 MHz, D₂O) δ 8.01 (d, J=8.2 Hz, 1H), 7.64 (d, J=8.5 Hz, 1H), 7.55 (s, 1H), 7.35-7.23 (m, 3H), 7.18 (d, J=6.8 Hz, 1H), 5.80 (d, J=7.4 Hz, 1H), 4.89 (d, J=7.2 Hz, 1H), 4.31 (s, 1H), 4.23 (d, J=3.4 Hz, 1H), 4.18-4.07 (m, 3H), 3.02 (d, J=8.3 Hz, 1H), 2.90 (d, J=8.0 Hz, 1H), 2.61 (d, J=9.3 Hz, 1H), 2.17-2.00 (m, 3H). (ESI) m/z=583.6 (M−1).

### Example S26

### Synthesis of (((((2R,3S,4R,5S)-5-(2-chloro-4-((4-hydroxycyclohexyl)amino)quinazolin-7-yl-3,4-dihydroxytetrahydrofuran-2-yl)methoxy)(hydroxy)phosphoryl)methyl)phosphonic acid

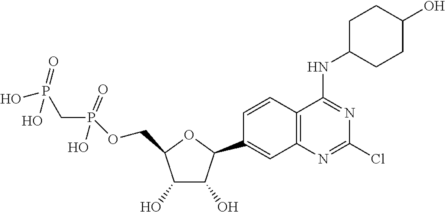 [SMILES: O=P(O)(O)CP(=O)(O)OC[C@H]1O[C@@H](c2ccc3c(NC4CCC(O)CC4)nc(Cl)nc3c2)[C@H](O)[C@@H]1O]

(((((2R,3S,4R,5S)-5-(2-chloro-4-((4-hydroxycyclohexyl)amino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with 4-aminocyclohexan-1-ol.

¹H NMR (400 MHz, D₂O) δ 7.96 (s, 1H), 7.62-7.45 (m, 2H), 4.85 (d, J=8.2 Hz, 1H), 4.32-4.03 (m, 6H), 3.66-3.60 (m, 1H),2.11-1.89 (m, 6H), 1.50-1.32 (m, 4H). Mass Spectrum (ESI) m/z=566.0 (M−1).

### Example S27

### Synthesis of [({[(2R,3S,4R,5S)-5-{2-chloro-4-[(4-methoxycyclohexyl)amino]quinazolin-7-yl}-3,4-dihydroxyoxolan-2-yl]methoxy}(hydroxy)phosphoryl)methyl]phosphonic acid

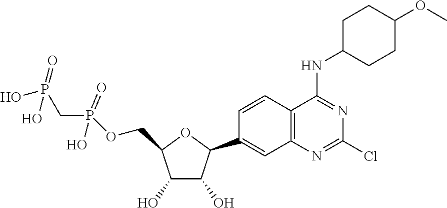 [SMILES: COC1CCC(Nc2nc(Cl)nc3cc([C@@H]4O[C@H](COP(=O)(O)CP(=O)(O)O)[C@@H](O)[C@H]4O)ccc23)CC1]

[({[(2R,3S,4R,5S)-5-{2-chloro-4-[(4-methoxycyclohexyl)amino]quinazolin-7-yl}-3,4-dihydroxyoxolan-2-yl]methoxy}(hydroxy)phosphoryl)methyl]phosphonic acid was synthesized by procedures similar to the ones described in Example S1, replacing benzylamine in Step G with 4-methoxycyclohexan-1-amine.

¹H NMR (400 MHz, D₂O) δ 8.01 (d, J=8.6 Hz, 1H), 7.66 (d, J=8.6 Hz, 1H), 7.49 (s, 1H), 4.87 (d, J=6.9 Hz, 1H), 4.35-4.29 (m, 1H), 4.24-4.18 (m, 1H), 4.15-4.01 (m, 4H), 3.38-3.29 (m, 4H), 2.14-1.90 (m, 6H), 1.49-1.26 (m, 4H). Mass Spectrum (ESI) m/z=580.0 (M−1).

### Example S28

### Synthesis of (((((2R,3S,4R,5S)-5-(2-chloro-4-(((3-hydroxycyclohexyl)methyl)amino)quinazolin-7-yl)-3,4-dihydroxytetrahydrofuran-2-yl)methoxy)(hydroxy)phosphoryl)methyl)phosphonic acid

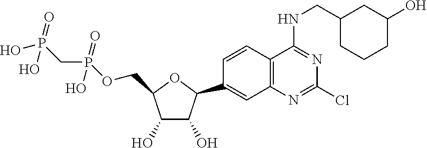 [SMILES: O=P(O)(O)CP(=O)(O)OC[C@H]1O[C@@H](c2ccc3c(NCC4CCCC(O)C4)nc(Cl)nc3c2)[C@H](O)[C@@H]1O]

(((((2R,3S,4R,5S)-5-(2-chloro-4-(((3-hydroxycyclohexyl)methyl)amino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with 3-(aminomethyl)cyclohexan-1-ol.

¹H NMR (400 MHz, D₂O) δ 7.98 (d, J=8.6 Hz, 1H), 7.74-7.56 (m, 1H), 7.49 (s, 1H), 4.86 (d, J=7.1 Hz, 1H), 4.36-4.26 (m, 1H), 4.25-4.18 (m, 1H), 4.17-4.06 (m, 3H), 3.58-3.50 (m, 1H), 3.38 (d, J=7.2 Hz, 2H), 2.15-1.93 (m, 4H), 1.86-1.63 (m, 4H), 1.13-0.99 (m, 2H), 0.88-0.80 (m, 1H). Mass Spectrum (ESI) m/z=582.0 (M−1).

### Example S29

### Synthesis of (((((2R,3S,4R,5S)-5-(2-chloro-4-(pyrrolidin-1-yl)quinazolin-7-yl)-3,4-dihydroxytetrahydrofuran-2-yl)methoxy)(hydroxy)phosphoryl)methyl)phosphonic acid

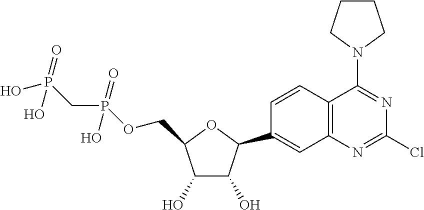 [SMILES: O=P(O)(O)CP(=O)(O)OC[C@H]1O[C@@H](c2ccc3c(N4CCCC4)nc(Cl)nc3c2)[C@H](O)[C@@H]1O]

(((((2R,3S,4R,5S)-5-(2-chloro-4-(pyrrolidin-1-yl)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with pyrrolidine.

¹H NMR (400 MHz, D₂O) δ 8.21 (d, J=8.8 Hz, 1H), 7.57-7.42 (m, 2H), 4.86 (d, J=6.7 Hz, 1H), 4.34-4.29 (m, 1H), 4.23-4.03 (m, 4H), 3.77-3.65 (m, 4H), 2.07-1.85 (m, 6H). Mass Spectrum (ESI) m/z=524.0 (M+1).

### Example S30

Synthesis of (((((2R,3S,4R,5S)-5-(2-chloro-4-(indolin-1-yl)quinazolin-7-yl)-3,4-dihydroxytetrahydrofuran-2-yl)methoxy)(hydroxy)phosphoryl)methyl)phosphonic acid

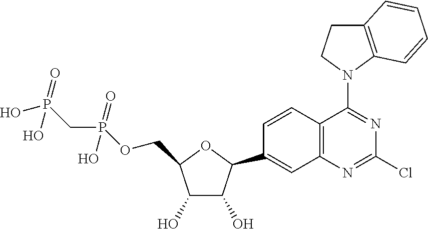 [SMILES: O=P(O)(O)CP(=O)(O)OC[C@H]1O[C@@H](c2ccc3c(N4CCc5ccccc54)nc(Cl)nc3c2)[C@H](O)[C@@H]1O]

(((((2R,3S,4R,5S)-5-(2-chloro-4-(indolin-1-yl)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with indoline.

¹H NMR (400 MHz, D₂O) δ 8.22-8.10 (m, 1H), 7.68-7.48 (m, 3H), 7.35-7.25(m, 1H), 7.20-7.11 (m, 1H), 7.10-7.05 (m, 1H), 4.93-4.85 (m, 1H), 4.50-4.35 (m, 2H), 4.32-4.20 (m, 2H), 4.17-4.03 (m, 3H), 3.19-3.13 (m, 2H), 2.10 (t, J=19.8 Hz, 2H). Mass Spectrum (ESI) m/z=570.0 (M−1).

### Example S31

### Synthesis of (((((2R,3S,4R,5S)-5-(2-chloro-4-(cyclohexylamino)quinazolin-7-yl)-3,4-dihydroxytetrahydrofuran-2-yl)methoxy)(hydroxy)phosphoryl)methyl)phosphonic acid

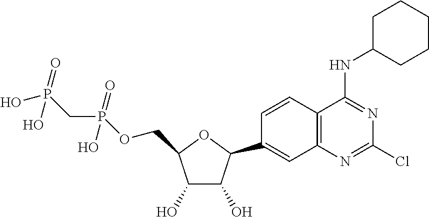 [SMILES: O=P(O)(O)CP(=O)(O)OC[C@H]1O[C@@H](c2ccc3c(NC4CCCCC4)nc(Cl)nc3c2)[C@H](O)[C@@H]1O]

(((((2R,3S,4R,5S)-5-(2-chloro-4-(cyclohexylamino)quinazolin-7-yl)-3,4-dihydroxytetrahydrofuran-2-yl)methoxy)(hydroxy)phosphoryl)methyl)phosphonic acid was synthesized by procedures similar to the ones described in Example S1, replacing benzylamine in Step G with cyclohexanamine.

¹H NMR (400 MHz, D₂O) δ 7.98 (d, J=8.6 Hz, 1H), 7.62 (d, J=8.7 Hz, 1H), 7.45 (s, 1H), 4.86 (d, J=6.8 Hz, 1H), 4.33-4.29 (m, 1H), 4.21-4.18 (m, 1H), 4.16-4.05 (m, 3H), 4.01-3.94 (m, 1H), 2.05-1.93 (m, 2H), 1.92-1.87 (m, 2H), 1.76-1.66 (m, 2H), 1.59 (d, J=12.3 Hz, 1H), 1.38-1.24 (m, 4H), 1.19-1.16 (m, 1H). Mass Spectrum (ESI) m/z=550.1 (M−1).

### Example S32

### Synthesis of (((((2R,3S,4R,5S)-5-(4-(benzylamino)-2-chloroquinazolin-8-yl)-3,4-dihydroxytetrahydrofuran-2-yl)methoxy)(hydroxy)phosphoryl)methyl)phosphonic acid

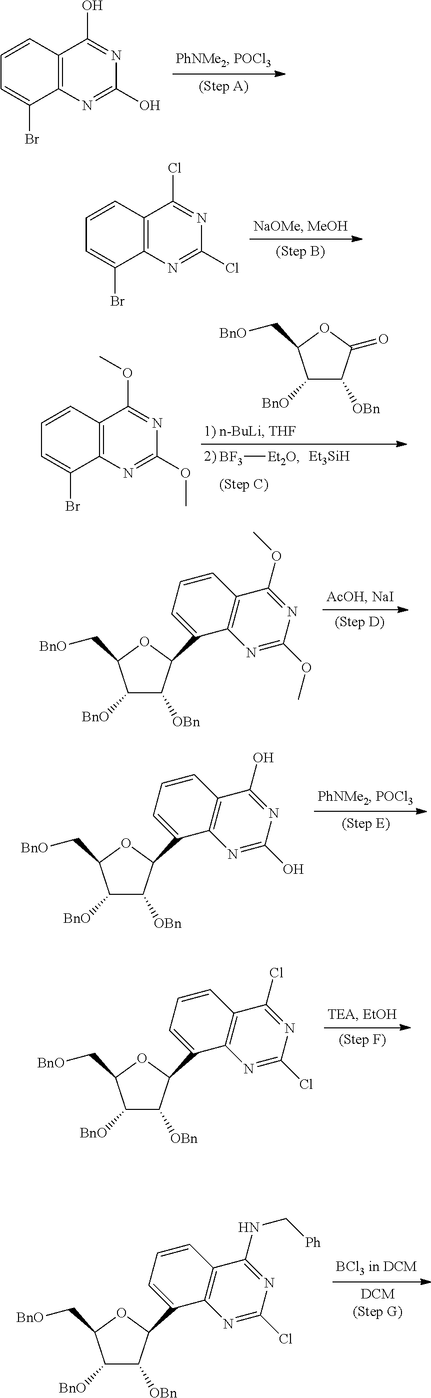

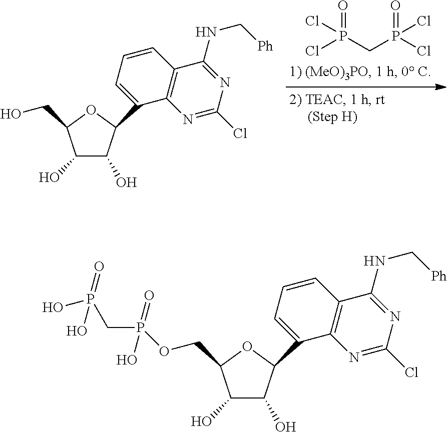 [SMILES: OC[C@H]1O[C@@H](c2cccc3c(NCc4ccccc4)nc(Cl)nc23)[C@H](O)[C@@H]1O ; O=P(Cl)(Cl)CP(=O)(Cl)Cl ; O=P(O)(O)CP(=O)(O)OC[C@H]1O[C@@H](c2cccc3c(NCc4ccccc4)nc(Cl)nc23)[C@H](O)[C@@H]1O]

Step A: To a mixture of 8-bromoquinazoline-2,4-diol (5 g, 20.8 mmol) in POCl₃ (50 mL) was added N,N-dimethylaniline (5 g, 41.7 mmol) carefully. The reaction was refluxed for 4 h. Then the reaction was concentrated and quenched with water, extracted with DCM (100 mL×2). The combined organic layers were washed with sat. NaHCO₃ solution and brine, dried over Na₂SO₄, filtered and the filtrate was concentrated and purified by CombiFlash® (20 g, eluting with PE/EA=10:1) to give 8-bromo-2,4-dichloroquinazoline as a pale-yellow solid (3.15 g, 95% yield). Mass Spectrum (ESI) m/z=276.9 (M+1).

Step B: A solution of 8-bromo-2,4-dichloroquinazoline (3.15 g, 11.4 mmol) in 228 mL of 0.5 M sodium methoxide in methanol was stirred under reflux conditions for 12 h, then allowed to cool to room temperature. The reaction mixture was placed in an ice bath and 1 M aq. HCl was added until precipitation occured and solution was slightly acidified to litmus, then filtered to obtain the solid. The captured solid was washed with cold water, dissolved with DCM, dried over anh. Na₂SO₄, filtered, the filtrate was concentrated and purified by CombiFlash® (20 g, PE/EA=5:1) to give 8-bromo-2,4-dimethoxyquinazoline as a pale-yellow solid (1.45 g, 51% yield). Mass Spectrum (ESI) m/z=268.9 (M+1).

Step C: To a solution of 8-bromo-2,4-dimethoxyquinazoline (1.45 g, 5.39 mmol) in 58 mL anhydrous THF was carefully added n-BuLi (2.4 M, 2.5 mL) dropwise at −78° C. under a nitrogen atmosphere. The reaction mixture was stirred for 30 min at −78° C., then a solution of (3R,4R,5R)-3,4-bis(benzyloxy)-5-[(benzyloxy)methyl]oxolan-2-one (1.9 g, 4.54 mmol) in 15 mL anh. THF was added dropwise over 30 min. The reaction was stirred for 2.5 h at −78° C., then for 30 min at −30° C. After quenching with sat aq. NH₄Cl solution (20 mL), the aqueous layer was extracted with EA (50 mL×3). The combined organic layers were dried over anhydrous Na₂SO₄, filtered and the filtrate was evaporated in vacuo. The dried, crude product was dissolved in anhydrous DCM (60 mL) and stirred at −78° C. To this mixture, triethylsilane (2.9 mL, 18 mmol) was added dropwise, followed by boron trifluoride diethyl etherate (2.2 mL, 18 mmol). The reaction was stirred overnight at −78° C. and allowed to warm up to rt. After quenching with sat aq NaHCO₃ solution, the aqueous layer was extracted with EA (50 mL×3). The combined organic layers were dried over anhydrous Na₂SO₄, filtered and the filtrated was concentrated in vacuo and purified by CombiFlash® (20 g, PE/EA=9:1) to give 8-[(2S,3S,4R,5R)-3,4-bis(benzyloxy)-5-[(benzyloxy)methyl]oxolan-2-yl]-2,4-dimethoxyquinazoline as a light yellow oil (1.4 g, 43% yield). Mass Spectrum (ESI) m/z=592.8 (M+1).

Step D: To a solution of 8-[(2S,3S,4R,5R)-3,4-bis(benzyloxy)-5-[(benzyloxy)methyl]oxolan-2-yl]-2,4-dimethoxyquinazoline (1.4 g, 2.36 mmol) in glacial acetic acid (20 mL) was added sodium iodide (1.65 g, 10.9 mmol). The reaction mixture was heated to 60° C. for 45 min and the volatiles were removed in vacuo. The residue was dissolved in EtOAc and extracted with aq. saturated Na₂SO₃ (20 mL×3) and saturated sodium bicarbonate solution (20 mL×3). The aqueous layers were extracted with EtOAc (20 mL×3). The combined organics were dried over Na₂SO₄, filtered and the filtrate was concentrated in vacuo. The residue was purified by CombiFlash® (12 g, PE/EA=3:1) to give 8-[(2S,3S,4R,5R)-3,4-bis(benzyloxy)-5-[(benzyloxy)methyl]oxolan-2-yl]quinazoline-2,4-diol as a white solid (1 g, 75% yield). Mass Spectrum (ESI) m/z=564.8 (M+1).

Step E: To a solution of 8-[(2S,3S,4R,5R)-3,4-bis(benzyloxy)-5-[(benzyloxy)methyl]oxolan-2-yl]quinazoline-2,4-diol (400 mg, 0.71 mmol) in phosphorus oxychloride (10 mL) was added N,N-dimethylaniline (168 mg, 1.39 mmol). The reaction mixture was stirred and heatd to reflux for 4 h. Then the solvent was removed in vacuo, the residue was diluted with DCM (80 mL) and washed with aq. saturated sodium bicarbonate (30 mL×3). The organic phase was dried over anhydrous sodium sulfate and concentrated under reduced pressure. The residue was purified by CombiFlash® (4 g, PE/EA=5:1) to give 8-[(2S,3S,4R,5R)-3,4-bis(benzyloxy)-5-[(benzyloxy)methyl]oxolan-2-yl]-2,4-dichloroquinazoline as a white solid (190 mg, 45% yield). Mass Spectrum (ESI) m/z=600.7 (M+1).

Step F: Benzylamine (34.2 mg, 0.32 mmol) and triethylamine (31 mg, 0.32 mmol) was added to a solution of 8-[(2S,3S,4R,5R)-3,4-bis(benzyloxy)-5-[(benzyloxy)methyl]oxolan-2-yl]-2,4-dichloroquinazoline (190 mg, 0.32 mmol) in EtOH (10 mL). The mixture was stirred and heated to 60° C. for 3 h. The solvent was removed under reduced pressure to give N-benzyl-8-[(2S,3S,4R,5R)-3,4-bis(benzyloxy)-5-[(benzyloxy)methyl]oxolan-2-yl]-2-chloroquinazolin-4-amine as a light yellow oil (200 mg, 96% yield) which was carried forward without further purification. Mass Spectrum (ESI) m/z=671.7 (M+1).

Step G: To a solution of N-benzyl-8-[(2S,3S,4R,5R)-3,4-bis(benzyloxy)-5-[(benzyloxy)methyl]oxolan-2-yl]-2-chloroquinazolin-4-amine (190 mg, 0.28 mmol) in DCM (4 mL) was added BCl₃ (1 M in DCM, 2.8 mL, 2.8 mmol) dropwise at −70° C. under a N₂ atmosphere. The reaction solution was stirred at −70° C. for 1 h. Then the reaction was brought to −30° C. over a period of 30 min, and quenched with a mixture of methanol: chloroform (2:1, 3 mL). After the reaction mixture reached rt, it was neutralized with NH₃ in methanol and concentrated. The residue was purified by CombiFlash® (4 g, eluting with DCM/MeOH=10:1) to give (2S,3R,4S,5R)-2-[4-(benzylamino)-2-chloroquinazolin-8-yl]-5-(hydroxymethyl)oxolane-3,4-diol as a white solid (95 mg, 80% yield). Mass Spectrum (ESI) m/z=401.9 (M+1).

Step H: To a solution of (2S,3R,4S,5R)-2-[4-(benzylamino)-2-chloroquinazolin-8-yl]-5-(hydroxymethyl)oxolane-3,4-diol (60 mg, 0.15 mmol) in trimethyl phosphate (1 mL) at 0° C. was added a cold solution of methylenebis(phosphonic dichloride) (186 mg, 0.75 mmol) in trimethyl phosphate (0.5 mL) dropwise under a N₂ atmosphere. Then the reaction solution was stirred at 0° C. for 4 h. Triethylammonium bicarbonate (0.5 M, 1 mL) was added to the reaction carefully and the reaction was stirred at this temperature for 15 mins, then warmed to room temperature and stirring was continued for 1 h. Trimethyl phosphate was extracted using tert-butyl methyl ether (5 mL×2) and the aqueous layer was basified with ammonium hydroxide to pH ˜7-8. Then the solution was purified by Prep-HPLC using a gradient of 0.2% ammonium hydroxide/ACN from 100:0 to 85:15, and suitable fractions were pooled and lyophilized to give (((((2R,3S,4R,5S)-5-(4-(benzylamino)-2-chloroquinazolin-8-yl)-3,4-dihydroxytetrahydrofuran-2-yl)methoxy)(hydroxy)phosphoryl)methyl)phosphonic acid (10 mg, 12% yield) as a white solid.

¹H NMR (400 MHz, DMSO-d6+D₂O) δ 8.27-8.18 (m, 1H), 8.09-8.03 (m, 1H), 7.52 (t, J=7.9 Hz, 1H), 7.41-7.28 (m, 4H), 7.28-7.20 (m, 1H), 5.64-5.48 (m, 1H), 4.85-4.68 (m, 2H), 4.67-4.54 (m, 1H), 4.54-4.38 (m, 1H), 4.54-4.38 (m, 1H), 4.21-4.06 (m, 1H), 4.04-3.88 (m, 1H), 3.87-3.76 (m, 1H), 1.88-1.60 (m, 2H). Mass Spectrum (ESI) m/z=557.9 (M−1).

### Example S33

### Synthesis of (((((2R,3S,4R,5S)-5-(2-chloro-4-(cyclopentyloxy)quinazolin-7-yl)-3,4-dihydroxytetrahydrofuran-2-yl) methoxy)(hydroxy)phosphoryl)methyl)phosphonic acid

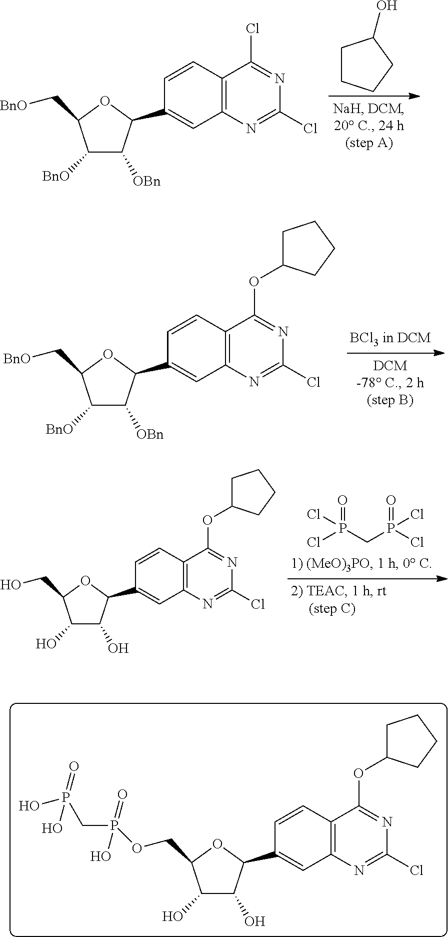 [SMILES: Clc1nc(Cl)c2ccc([C@@H]3O[C@H](COCc4ccccc4)[C@@H](OCc4ccccc4)[C@H]3OCc3ccccc3)cc2n1 ; OC1CCCC1 ; Clc1nc(OC2CCCC2)c2ccc([C@@H]3O[C@H](COCc4ccccc4)[C@@H](OCc4ccccc4)[C@H]3OCc3ccccc3)cc2n1 ; OC[C@H]1O[C@@H](c2ccc3c(OC4CCCC4)nc(Cl)nc3c2)[C@H](O)[C@@H]1O ; O=P(Cl)(Cl)CP(=O)(Cl)Cl ; O=P(O)(O)CP(=O)(O)OC[C@H]1O[C@@H](c2ccc3c(OC4CCCC4)nc(Cl)nc3c2)[C@H](O)[C@@H]1O]

Step A: To a mixture of cyclopentanol (286 mg, 3.32 mmol) in DCM (15 mL) was added sodium hydride (73 mg, 1.83 mmol, 60%) carefully under nitrogen. The reaction was stirred for 30 min at 20° C. Then a solution of 7-[(2S,3S,4R,5R)-3,4-bis(benzyloxy)-5-[(benzyloxy)methyl]oxolan-2-yl]-2,4-dichloroquinazoline (998 mg, 1.66 mmol) in DCM (5 mL) was added at 20° C. under nitrogen. The reaction was stirred for 24 h at 20° C. The reaction was quenched by sat. NH₄Cl solution and extracted with EA (10 mL×3). The organic layer was washed with brine and dried over Na₂SO₄, filtered and the filtrate was concentrated. The crude product was purified by silica gel column chromatography (24 g, PE/EA=5:1) to give 7-[(2S,3S,4R,5R)-3,4-bis(benzyloxy)-5-[(benzyloxy)methyl]oxolan-2-yl]-2-chloro-4-(cyclopentyloxy)quinazoline (600 mg, yield 50%) as a yellow oil. Mass Spectrum (ESI) m/z=651.1 (M+1).

Step B: To a mixture of 7-[(3S,4R,5R)-3,4-bis(benzyloxy)-5-[(benzyloxy)methyl]oxolan-2-yl]-2-chloro-4-(cyclopentyloxy) quinazoline (500 mg, 0.77 mmol) in DCM (10 mL) was added boron trichloride (7.7 mL, 7.7 mmol, 1M) carefully at −78° C. under nitrogen. The reaction was stirred for 2 h at −78° C. The reaction was quenched by solution of NH₃ in MeOH (7M) and stirred for 2 h. The organic layer was concentrated. The crude product was purified by silica gel column chromatography (12 g, DCM/MeOH=10:1) to give (2S,3R,4S,5R)-2-[2-chloro-4-(cyclopentyloxy) quinazolin-7-yl]-5-(hydroxymethyl)oxolane-3,4-diol (200 mg, yield 61%) as a white solid. Mass Spectrum (ESI) m/z=381.1 (M+1).

Step C: To a mixture of (2S,3R,4S,5R)-2-[2-chloro-4-(cyclopentyloxy)quinazolin-7-yl]-5-(hydroxymethyl)oxolane-3,4-diol (100 mg, 0.26 mmol) in trimethyl phosphate (1 mL) was added a solution of methylenebis(phosphonic dichloride) (325 mg, 1.3 mmol) in trimethyl phosphate (0.5 mL) carefully at 0° C. under nitrogen. The reaction was stirred for 1 h at 0° C. TEAC (0.5 M, 1.5 mL) was added to the reaction carefully and the reaction was stirred at this temperature for 15 mins, then warmed to room temperature and continued to stir for 1 h. Trimethyl phosphate was extracted using tert-butyl methyl ether (5 mL×3) and the aqueous layer was basified with ammonium hydroxide to pH ˜7-8. Then the solution was purified by Prep-HPLC using a gradient of 0.2% TEAC in water/ACN from 90:10 to 60:40 to give [({[(2R,3S,4R,5S)-5-[2-chloro-4-(cyclopentyloxy)quinazolin-7-yl]-3,4-dihydroxyoxolan-2-yl]methoxy}(hydroxy)phosphoryl)methyl]phosphonic acid (12 mg, yield 7.7%) as a white solid.

¹H NMR (400 MHz, D₂O) δ 8.17 (d, J=8.5 Hz, 1H), 7.71-7.67 (m, 2H), 5.62-5.52 (m, 1H), 4.92 (d, J=7.4 Hz, 1H), 4.35-4.21 (m, 2H), 4.19-4.05 (m, 3H), 2.12 (t, J=19.8 Hz, 2H), 1.98-1.93 (m, 4H), 1.79-1.76 (m, 2H), 1.70-1.57 (m, 2H). Mass Spectrum (ESI) m/z=539.0 (M+1).

### Example S34

### Synthesis of (((((2R,3R,4R,5S)-5-(4-(benzylamino)-2-chloroquinazolin-7-yl)-3,4-dihydroxy-4-methyltetrahydrofuran-2-yl)methoxy)(hydroxy)phosphoryl)methyl)phosphonic acid

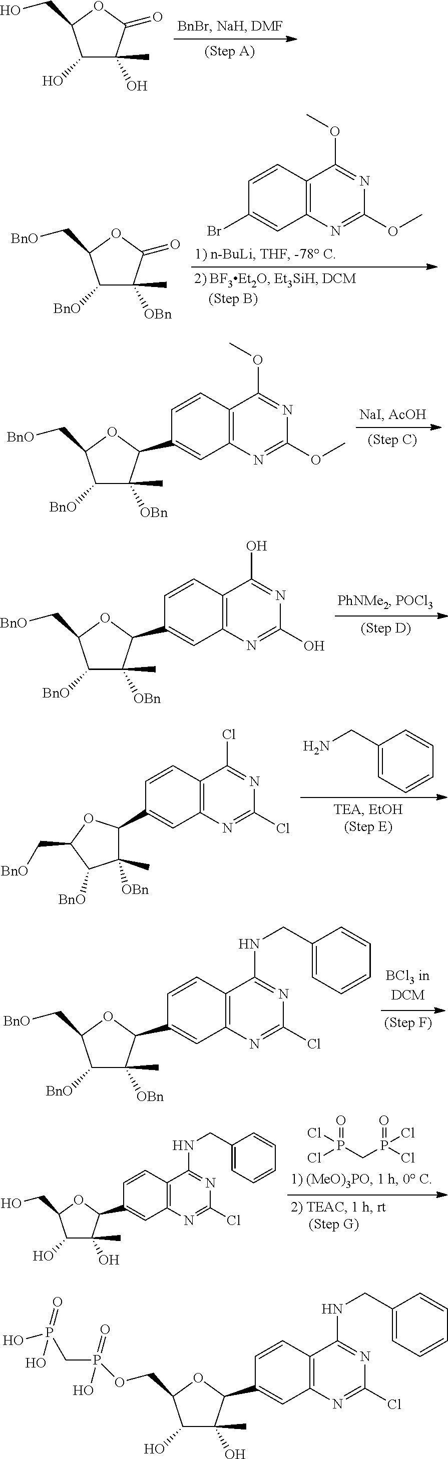

Step A: To a solution of (3R,4R,5R)-3,4-dihydroxy-5-(hydroxymethyl)-3-methyloxolan-2-one (5 g, 30.9 mmol) in DMF (70 mL) was added NaH (60%, 4.3 g, 108.2 mmol) at 0° C. under an atmosphere of N₂. After 1 h, (bromomethyl)benzene (21 g, 123.5 mmol) was added to the reaction dropwise. Then the reaction mixture was stirred at 5-10° C. for 3 h. The mixture was poured into ice-water slowly and extracted with EA (30 mL×3). The combined organic layers were washed with water and brine, dried, filtered, the filtrate was concentrated and purified by CombiFlash® (eluting with PE/EA=10:1) to give (3R,4R,5R)-3,4-bis(benzyloxy)-5-[(benzyloxy)methyl]-3-methyloxolan-2-one (6.1 g, 45% yield) as a colorless oil. ¹H NMR (400 MHz, CDCl₃) δ 7.39-7.29 (m, 15H), 4.81 (d, J=11.7 Hz, 1H), 4.69 (d, J=4.3 Hz, 2H), 4.67-4.50 (m, 4H), 4.07 (d, J=7.6 Hz, 1H), 3.82 (dd, J=11.5, 2.4 Hz, 1H), 3.63 (dd, J=11.5, 3.5 Hz, 1H), 1.55 (s, 3H).

Step B: To a solution of 7-bromo-2,4-dimethoxyquinazoline (3.5 g, 13.1 mmol) in 55 mL. anhydrous THF stirring at −78° C., 7.1 mL of 2.4 M n-butyllithium solution in pentane (17 mmol) was carefully added dropwise. The reaction mixture was stirred for 30 min. at −78° C., then a solution of (3R,4R,5R)-3,4-bis(benzyloxy)-5-[(benzyloxy)methyl]-3-methyloxolan-2-one (5.6 g, 13.1 mmol) in 10 mL anhy. THF was added dropwise. The reaction was stirred for 2.5 h at −78° C., then for 30 min. at −30° C. After quenching with sat. aq. NH₄Cl solution at −30° C., the mixture was extracted with Et₂O. The organic layer was dried over anhydrous Na₂SO₄, filtered, and the filtrate was concentrated under reduced pressure. The dried, crude product was dissolved in anhydrous CH₂C₂ and stirred at −78° C. To this mixture, 6.1 g of triethylsilane (52.4 mmol) was added dropwise, followed by 15.5 g of boron trifluoride diethyl etherate (52.4 mmol). The reaction was stirred overnight at room temperature. After quenching with sat. aq. NaHCO₃ solution, the mixture was extracted with Et₂O, the organic layer was dried over anhydrous Na₂SO₄, filtered, concentrated and purified by CombiFlash® (40 g, PE/EA=3:1) to give 7-[(3S,4R,5R)-3,4-bis(benzyloxy)-5-[(benzyloxy)methyl]-3-methyloxolan-2-yl]-2,4-dimethoxyquinazoline (1.9 g, 24% yield) as a white solid. Mass Spectrum (ESI) m/z=606.8 (M+1).

Step C: To a solution of 7-[(3S,4R,5R)-3,4-bis(benzyloxy)-5-[(benzyloxy)methyl]-3-methyloxolan-2-yl]-2,4-dimethoxyquinazoline (1.9 g, 3.14 mmol) in glacial acetic acid (30 mL) was added sodium iodide (2.3 g, 15.7 mmol). The reaction mixture was heated to 60° C. for 45 min, and then the volatiles were removed in vacuo. The residue was dissolved in EtOAc and washed with aq. saturated Na₂SO₃ (20 mL×2) and saturated sodium bicarbonate solution (20 mL×2). The aqueous layers were extracted with EtOAc (30 mL×3). The combined organics were dried over Na₂SO₄ and concentrated in vacuo. The residue was purified by CombiFlash® (PE/EA=1:1) to give 7-[(3S,4R,5R)-3,4-bis(benzyloxy)-5-[(benzyloxy)methyl]-3-methyloxolan-2-yl]quinazoline-2,4-diol (1.2 g, 66% yield) as a solid. Mass Spectrum (ESI) m/z=578.8 (M+1).

Step D: 7-[(3S,4R,5R)-3,4-bis(benzyloxy)-5-[(benzyloxy)methyl]-3-methyloxolan-2-yl]quinazoline-2,4-diol (1.2 g, 2.08 mmol) was added to POCl₃ (20 mL), then N,N-dimethylaniline (504 mg, 4.16 mmol) was added carefully. The reaction was stirred at 90° C. for 4 h. The solvent was removed under reduced pressure and the residue was diluted with DCM, poured into ice-water slowly. The organic layer was washed with water and brine, dried, concentrated and purified by CombiFlash® (PE/EA=1:1) to give 7-[(3S,4R,5R)-3,4-bis(benzyloxy)-5-[(benzyloxy)methyl]-3-methyloxolan-2-yl]-2,4-dichloroquinazoline (500 mg, 33.2% yield) as a yellow oil. Mass Spectrum (ESI) m/z=637.0 (M+23).

Step E: To a solution of 7-[(3S,4R,5R)-3,4-bis(benzyloxy)-5-[(benzyloxy)methyl]-3-methyloxolan-2-yl]-2,4-dichloroquinazoline (500 mg, 0.8 mmol) in ethanol (10 mL) was added benzylamine (105 mg, 0.96 mmol) and DIPEA (205 mg, 1.6 mmol). Then the reaction was refluxed for 4h. The solution was concentrated and purified by CombiFlash® (PE/EA=2:1) to give N-benzyl-7-[(3S,4R,5R)-3,4-bis(benzyloxy)-5-[(benzyloxy)methyl]-3-methyloxolan-2-yl]-2-chloroquinazolin-4-amine (400 mg, 73% yield) as a yellow solid. Mass Spectrum (ESI) m/z=685.7 (M+1).

Step F: To a solution of N-benzyl-7-[(3S,4R,5R)-3,4-bis(benzyloxy)-5-[(benzyloxy)methyl]-3-methyloxolan-2-yl]-2-chloroquinazolin-4-amine (400 mg, 0.58 mmol) in DCM (6 mL) at −78° C. was added BCl₃ in DCM (1M, 5.8 mL, 5.8 mmol) dropwise. Then the reaction was stirred at this temperature for 1 h. DCM/MeOH (5 mL, 1:1) was added to the reaction carefully and the reaction was stirred for 30 min. Then neutralized with NH₃ in methanol to pH=7˜8. The resulting mixture was concentrated and purified by CombiFlash® (DCM/MeOH=10:1) to give (3R,4R,5R)-2-[4-(benzylamino)-2-chloroquinazolin-7-yl]-5-(hydroxymethyl)-3-methyloxolane-3,4-diol (200 mg, 83%) as a solid. Mass Spectrum (ESI) m/z=415.8 (M+1).

Step G: To a solution of (3R,4R,5R)-2-[4-(benzylamino)-2-chloroquinazolin-7-yl]-5-(hydroxymethyl)-3-methyloxolane-3,4-diol (100 mg,0.24 mmol) in trimethyl phosphate (1.5 mL) at 0° C. was added a cold solution of methylenebis(phosphonic dichloride) (299 mg, 1.2 mmol) in trimethyl phosphate (1 mL) dropwise. Then the reaction solution was stirred at 0° C. for 1 h. TEAC (0.5 M, 1.7 mL) was added to the reaction carefully and the reaction was stirred at this temperature for 15 mins, then warmed to rt and continued to stir for 1 h. Trimethyl phosphate was extracted using tert-butyl methyl ether (5 mL×2) and the aqueous layer was basified with ammonium hydroxide to pH=7˜8, then purified by Prep-HPLC using a gradient of 0.2% formic acid in water/ACN from 85:15 to 60:40, and suitable fractions were pooled and lyophilized to give [({[(2R,3R,4R,5S)-5-[4-(benzylamino)-2-chloroquinazolin-7-yl]-3,4-dihydroxy-4-methyloxolan-2-yl]methoxy}(hydroxy)phosphoryl)methyl]phosphonic acid (45 mg, 33% yield) as a white solid.

¹H NMR (400 MHz, D₂O) δ 8.00 (d, J=6.0 Hz, 1H), 7.76 (s, 1H), 7.53 (d, J=7.5 Hz, 1H), 7.44-7.10 (m, 5H), 4.93 (s, 1H), 4.76 (s, 2H), 4.35-4.12 (m, 2H), 4.11-3.86 (m, 2H), 2.33-2.11 (m, 2H), 0.76 (s, 3H). Mass Spectrum (ESI) m/z=573.6 (M+1).

### Example S35

### Synthesis of (((((2R,3S,4R,5S)-5-(2-chloro-4-(3-phenylpyrrolidin-1-yl)quinazolin-7-yl)-3,4-dihydroxytetrahydrofuran-2-yl)methoxy)(hydroxy)phosphoryl)methyl)phosphonic acid

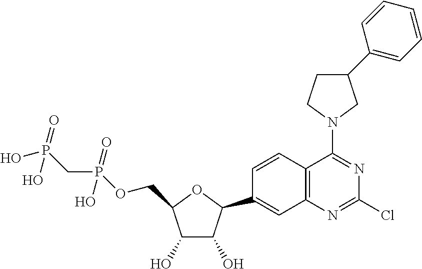 [SMILES: O=P(O)(O)CP(=O)(O)OC[C@H]1O[C@@H](c2ccc3c(N4CCC(c5ccccc5)C4)nc(Cl)nc3c2)[C@H](O)[C@@H]1O]

(((((2R,3S,4R,5S)-5-(2-chloro-4-(3-phenylpyrrolidin-1-yl)quinazolin-7-yl)-3,4-dihydroxytetrahydrofuran-2-yl)methoxy)(hydroxy)phosphoryl)methyl)phosphonic acid was prepared by procedures similar to the one described in Example S1, replacing benzylamine in Step G with 3-phenylpyrrolidine.

¹H NMR (400 MHz, D₂O) δ 7.99 (s, 1H), 7.58-7.33 (m, 2H), 7.30-7.24 (m, 2H), 7.23-7.15 (m, 3H), 4.78 (d, J=7.0 Hz, 1H), 4.23-4.15 (m, 2H), 4.09-4.00 (m, 3H), 3.97-3.55 (m, 3H), 3.49-3.28 (m, 2H), 2.42-2.24 (m, 1H), 2.12 (t, J=19.8 Hz, 2H), 1.99-1.90 (m, 1H). Mass Spectrum (ESI) m/z=599.7(M+1).

### Example S36

### Synthesis of [({[(2R,3S,4R,5S)-5-[2-chloro-4-(3,4-dihydro-1H-isoquinolin-2-yl)quinazolin-7-yl]-3,4-dihydroxyoxolan-2-yl]methoxy}(hydroxy)phosphoryl)methyl]phosphonic acid

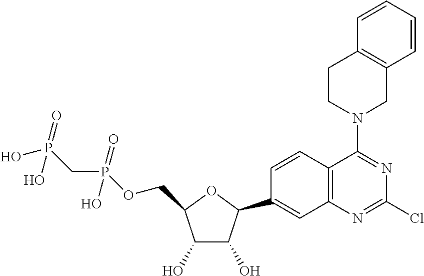 [SMILES: O=P(O)(O)CP(=O)(O)OC[C@H]1O[C@@H](c2ccc3c(N4CCc5ccccc5C4)nc(Cl)nc3c2)[C@H](O)[C@@H]1O]

[({[(2R,3S,4R,5S)-5-[2-chloro-4-(3,4-dihydro-1H-isoquinolin-2-yl)quinazolin-7-yl]-3,4-dihydroxyoxolan-2-yl]methoxy}(hydroxy)phosphoryl)methyl]phosphonic acid was prepared by procedures similar to the one described in Example S1, replacing benzylamine in Step G with 1,2,3,4-tetrahydroisoquinoline.

¹H NMR (400 MHz, D₂O) δ 8.06-8.02 (m, 1H), 7.60-7.52 (m, 1H), 7.51-7.45 (m , 1H), 7.18-7.02 (m, 4H), 4.82-4.75 (m, 3H), 4.32-4.29 (m, 1H), 4.19-4.10 (m, 1H), 4.09-4.06 (m, 3H), 3.95-3.90 (m, 2H), 2.99-2.90 (m, 2H), 1.97 (t, J=19.5 Hz, 2H). Mass Spectrum (ESI) m/z=586.0 (M+1).

### Example S37

### Synthesis of (((((2R,3S,4R,5S)-5-(2-chloro-4-((S)-tetrahydrofuran-3-yl)amino)quinazolin-7-yl)-3,4-dihydroxytetrahydrofuran-2-yl)methoxy)(hydroxy)phosphoryl)methyl)phosphonic acid

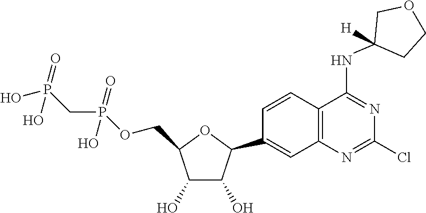 [SMILES: O=P(O)(O)CP(=O)(O)OC[C@H]1O[C@@H](c2ccc3c(N[C@H]4CCOC4)nc(Cl)nc3c2)[C@H](O)[C@@H]1O]

(((((2R,3S,4R,5S)-5-(2-chloro-4-(((S)-tetrahydrofuran-3-yl)amino)quinazolin-7-yl)-3,4-dihydroxytetrahydrofuran-2-yl)methoxy)(hydroxy)phosphoryl)methyl)phosphonic acid was prepared by procedures similar to the one described in Example S1, replacing benzylamine in Step G with (S)-tetrahydrofuran-3-amine.

¹H NMR (400 MHz, D₂O) δ 7.93 (d, J=8.8 Hz, 1H), 7.58 (d, J=8.8 Hz, 1H), 7.45 (s, 1H), 4.85-4.83 (m, 1H), 4.66-4.63 (m, 1H), 4.28-4.21 (m, 2H), 4.18-4.05 (m, 3H), 4.03-3.92 (m, 2H), 3.91-3.74 (m, 2H), 2.34 (dd, J=13.1, 7.5 Hz, 1H), 2.22-1.98 (m, 3H). Mass Spectrum (ESI) m/z=540.0 (M+1).

### Example S38

### Synthesis of (((((2R,3S,4R,5S)-5-(2-chloro-4-((R)-tetrahydrofuran-3-yl)amino)quinazolin-7-yl)-3,4-dihydroxytetrahydrofuran-2-yl)methoxy)(hydroxy)phosphoryl)methyl)phosphonic acid

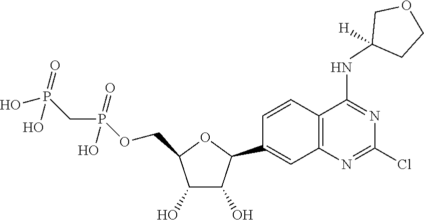 [SMILES: O=P(O)(O)CP(=O)(O)OC[C@H]1O[C@@H](c2ccc3c(N[C@@H]4CCOC4)nc(Cl)nc3c2)[C@H](O)[C@@H]1O]

(((((2R,3S,4R,5S)-5-(2-chloro-4-(((R)-tetrahydrofuran-3-yl)amino)quinazolin-7-yl)-3,4-dihydroxytetrahydrofuran-2-yl)methoxy)(hydroxy)phosphoryl)methyl)phosphonic acid was prepared by procedures similar to the one described in Example S1, replacing benzylamine in Step G with (R)-tetrahydrofuran-3-amine.

¹H NMR (400 MHz, D₂O) δ 8.00 (d, J=8.5 Hz, 1H), 7.64 (d, J=8.6 Hz, 1H), 7.50 (s, 1H), 4.90-4.86 (m, 1H), 4.56-4.46 (m, 1H), 4.33-4.28 (m, 1H), 4.25-4.21 (m, 1H), 4.19-4.07 (m, 3H), 4.06-3.95 (m, 2H), 3.94-3.79 (m, 2H), 2.40-2.34 (m, 1H), 2.21-1.96 (m, 3H). Mass Spectrum (ESI) m/z=540.0 (M+1).

### Example S39

### Synthesis of (((((2R,3S,4R,5S)-5-(4-(cyclopentylamino)-2-cyclopropylquinazolin-7-yl)-3,4-dihydroxytetrahydrofuran-2-yl)methoxy)(hydroxy)phosphoryl)methyl)phosphonic acid

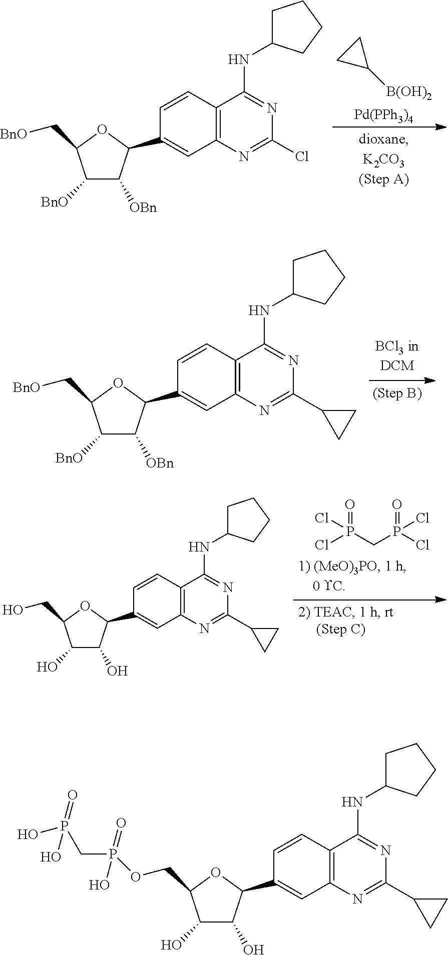

7-[(2S,3S,4R,5R)-3,4-bis(benzyloxy)-5-[(benzyloxy)methyl]oxolan-2-yl]-2-chloro-N-cyclopentylquinazolin-4-amine was synthesized by procedures similar to the ones described in Example S1, Steps A - G, replacing benzylamine in Step G with with cyclopentanamine.

Step A: To a solution of 7-[(2S,3S,4R,5R)-3,4-bis(benzyloxy)-5-[(benzyloxy)methyl]oxolan-2-yl]-2-chloro-N-cyclopentylquinazolin-4-amine (1 g, 1.5 mmol) in dioxane (10 mL) was added potassium carbonate (425 mg, 3 mmol), cyclopropylboronic acid (265 mg, 3 mmol) and tetrakis(triphenylphosphine)palladium (88 mg, 0.07 mmol). The reaction was stirred at 140° C. for 3 h in a microwave reactor. Then the reaction mixture was concentrated and purified by CombiFlash® (20 g, gradient elution, EA/PE=0-30%) to give 7-[(2S,3S,4R,5R)-3,4-bis(benzyloxy)-5-[(benzyloxy)methyl]oxolan-2-yl]-N-cyclopentyl-2-cyclopropylquinazolin-4-amine (1.12 g, 82% yield) as a yellow oil. Mass Spectrum (ESI) m/z=656.2 (M+1).

Step B and C: 7-((2S,3S,4R,5R)-3,4-bis(benzyloxy)-5-((benzyloxy)methyl)tetrahydrofuran-2-yl)-N-cyclopentyl-2-cyclopropylquinazolin-4-amine was converted to the title compound by procedures similar to the ones described in Example S1 Step H and Step I.

¹H NMR (400 MHz, D₂O) δ 7.93 (d, J=8.5 Hz, 1H), 7.69 (s, 1H), 7.49 (d, J=8.6 Hz, 1H), 4.86 (d, J=6.6 Hz, 1H), 4.52-4.43 (m, 1H), 4.34-4.28 (m, 1H), 4.24-4.18 (m, 1H), 4.17-4.06 (m, 3H), 2.12-1.94 (m, 5H), 1.73-1.62 (m, 2H), 1.60-1.45 (m, 4H), 1.14-1.11 (m, 2H), 1.07-1.01 (m, 2H). Mass Spectrum (ESI) m/z=572.1 (M−1).

### Example S40

### Synthesis of (((((2R,3S,4R,5S)-5-(2-chloro-4-(cyclopentylamino)pyrido[3,2-d]pyrimidin-7-yl)-3,4-dihydroxytetrahydrofuran-2-yl)methoxy)(hydroxy)phosphoryl)methyl)phosphonic acid

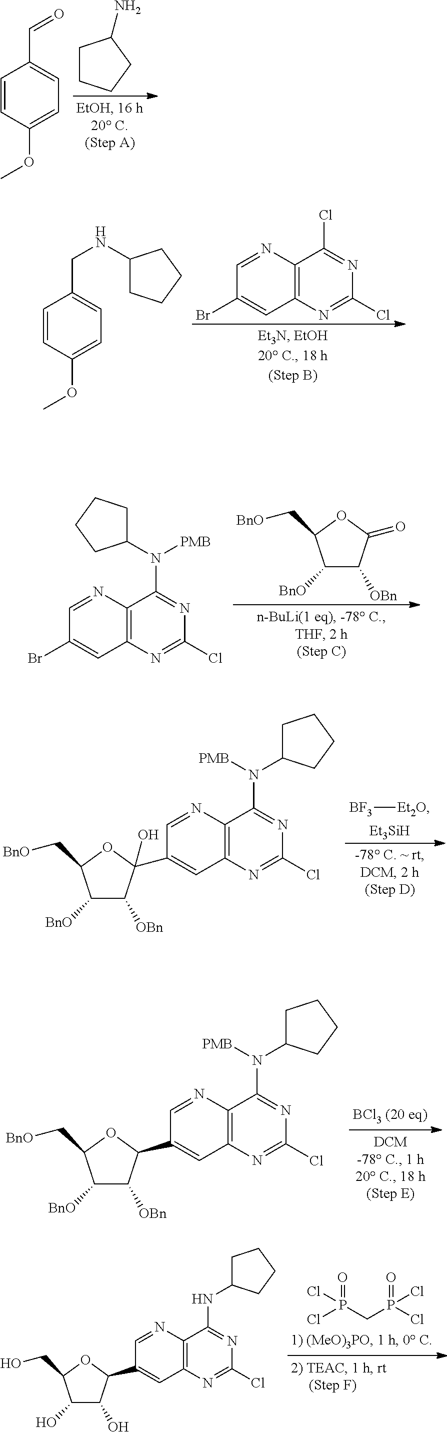

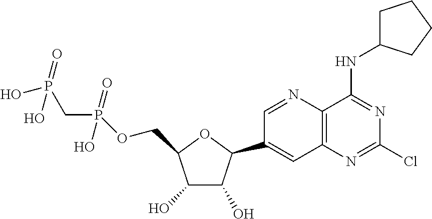 [SMILES: O=P(O)(O)CP(=O)(O)OC[C@H]1O[C@@H](c2cnc3c(NC4CCCC4)nc(Cl)nc3c2)[C@H](O)[C@@H]1O]

Step A: To a mixture of 4-methoxybenzaldehyde (3 g, 22.05 mmol) and cyclopentanamine (2.06 g, 24.26 mmol) in EtOH (100 mL) was added sodium borohydride (1.25 g, 33.08 mmol) carefully. The reaction was stirred for 16 h at 20° C. Then the reaction was quenched by sat. NH₄Cl solution (10 mL), filtered and the filtrate was concentrated. The crude product was purified by silica gel column chromatography (120 g, DCM/MeOH=20:1) to give N-[(4-methoxyphenyl)methyl]cyclopentanamine (3 g, 39.8% yield) as a white solid. Mass Spectrum (ESI) m/z=206.0 (M+1).

Step B: To a solution of 7-bromo-2,4-dichloropyrido[3,2-d]pyrimidine (1.36 g, 4.87 mmol) and triethylamine (986 mg, 9.74 mmol) in EtOH (50 mL) stirred under nitrogen was added 7-bromo-2,4-dichloropyrido[3,2-d] pyrimidine (1 g, 4.87 mmol). The reaction mixture was stirred at 20° C. for 18 h. Then the reaction was concentrated and purified by silica gel column chromatography (40 g, PE/EA=10:1) to give 7-bromo-2-chloro-N-cyclopentyl-N-[(4-methoxyphenyl)methyl]pyrido[3,2-d]pyrimidin-4-amine (1.5 g, 62.0% yield) as a yellow oil. Mass Spectrum (ESI) m/z=447.0 (M+1).

Step C: To a solution of 7-bromo-2-chloro-N-cyclopentyl-N-(2-methoxy-5-methylphenyl)pyrido[3,2-d]pyrimidin-4-amine (1.4 g, 3.13 mmol) in THF (15 mL) under an atmosphere of nitrogen was added n-butyllithium (1.43 mL, 3.44 mmol, 2.4 M) at −78° C. The reaction was stirred for 30 min at −78° C. Then a solution of (3R,4R,5R)-3,4-bis(benzyloxy)-5-[(benzyloxy)methyl]oxolan-2-one (1.44 g, 3.44 mmol) in THF (5 mL) was added at −78° C. under nitrogen. The reaction was stirred for 2 h at −78° C. The reaction was quenched by addn. of sat. NH₄Cl solution and extracted with EA (50 mL×3). The combined organic layers were washed with brine and dried over Na₂SO₄, filtered and the filtrate was concentrated. The crude product was purified by silica gel column chromatography (40 g, PE/EA=3:1) to give (3R,4R,5R)-3,4-bis(benzyloxy)-5-[(benzyloxy)methyl]-2-{2-chloro-4-[cyclopentyl(2-methoxy-5-methylphenyl)amino]pyrido[3,2-d]pyrimidin-7-yl}oxolan-2-ol (1.4 g, 51.1% yield) as a yellow oil. Mass Spectrum (ESI) m/z=787.1 (M+1).

Step D: To a solution of (3R,4R,5R)-3,4-bis(benzyloxy)-5-[(benzyloxy)methyl]-2-{2-chloro-4-[cyclopentyl(2-methoxy-5-methylphenyl)amino]pyrido[3,2-d]pyrimidin-7-yl}oxolan-2-ol (1.4 g, 1.78 mmol) in DCM (30 mL) under an atmosphere of nitrogen was added boron trifluoride etherate (2.2 g, 7.10 mmol, 46%) and triethylsilane (826 mg, 7.10 mmol) at −78° C. The reaction was stirred for 1 h at −78° C. and an additional 1 h at 20° C. Then the reaction was quenched by the addn. of sat. NaHCO₃ solution and extracted with DCM (10 mL×3). The combined organic layers were washed with brine and dried over Na₂SO₄, filtered and the filtrate was concentrated. The crude product was purified by CombiFlash® (20 g, PE/EA=5:1) to give 7-[(2S,3S,4R,5R)-3,4-bis(benzyloxy)-5-[(benzyloxy)methyl]oxolan-2-yl]-2- chloro-N-cyclopentyl-N-(2-methoxy-5-methylphenyl)pyrido[3,2-d]pyrimidin-4-amine (1 g, 65.9% yield) as a yellow oil. Mass Spectrum (ESI) m/z=771.2 (M+1).

Step E and Step F: 7-[(3S,4R,5R)-3,4-bis(benzyloxy)-5-[(benzyloxy)methyl]oxolan-2-yl]-2-chloro-N-cyclopentyl-N-(2-methoxy-5-methylphenyl)pyrido[3,2-d]pyrimidin-4-amine was converted to the title compound by procedures similar to the ones described in Example S1 Step H and Step I.

¹H NMR (400 MHz, D₂O) δ 8.77 (s, 1H), 7.88 (s, 1H), 4.94 (d, J=6.7 Hz, 1H), 4.36-4.33 (m, 2H), 4.28-4.03 (m, 4H), 2.11-1.87 (m, 4H), 1.72-1.60 (m, 6H). Mass Spectrum (ESI) m/z=539.0 (M+1).

### Example S41

### Synthesis of (((((2R,3S,4R,5S)-5-(2-chloro-4-(cyclopentyl(methyl)amino)pyrido[3,2-d]pyrimidin-7-yl)-3,4-dihydroxytetrahydrofuran-2-yl)methoxy)(hydroxy)phosphoryl)methyl)phosphonic acid

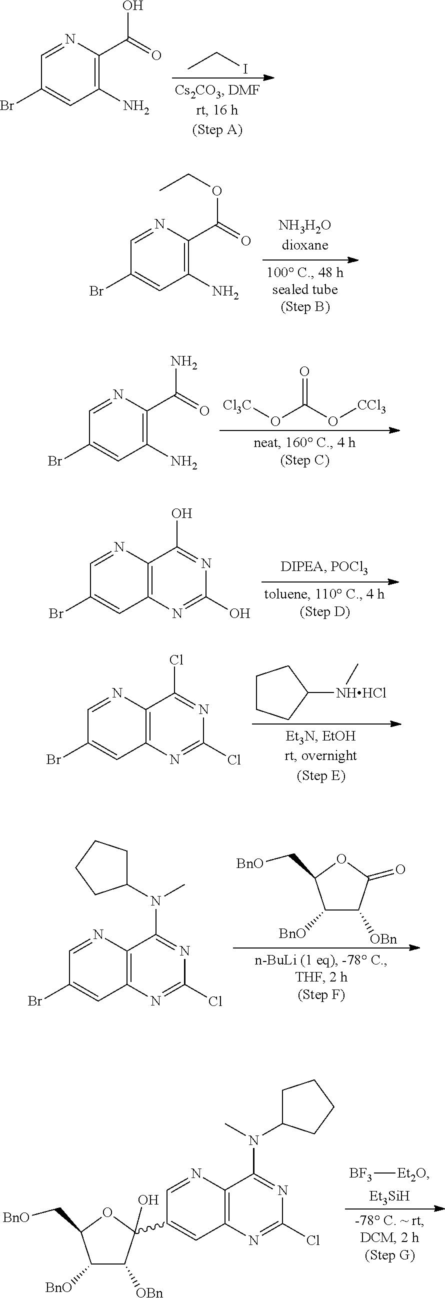

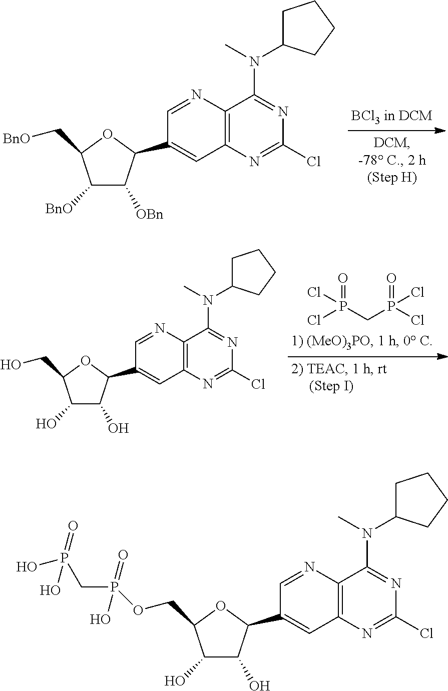 [SMILES: CN(c1nc(Cl)nc2cc([C@@H]3O[C@H](COCc4ccccc4)[C@@H](OCc4ccccc4)[C@H]3OCc3ccccc3)cnc12)C1CCCC1 ; CN(c1nc(Cl)nc2cc([C@@H]3O[C@H](CO)[C@@H](O)[C@H]3O)cnc12)C1CCCC1 ; O=P(Cl)(Cl)CP(=O)(Cl)Cl ; CN(c1nc(Cl)nc2cc([C@@H]3O[C@H](COP(=O)(O)CP(=O)(O)O)[C@@H](O)[C@H]3O)cnc12)C1CCCC1]

Step A: To a mixture of 3-amino-5-bromopyridine-2-carboxylic acid (45 g, 0.21 mol) and cesium carbonate (89 g, 0.27 mol) in DMF (400 mL) iodoethane (34.39 g, 0.22 mol) was added dropwise. The reaction was stirred for 16 h at rt. Then the reaction was diluted with EA (1500 mL), washed with sat. Na₂S₂O₃ solution and brine. The organic layer was dried over Na₂SO₄, filtered and the filtrate was concentrated. The crude product was purified by silica gel column chromatography (400 g, PE/EA=2:1) to give ethyl 3-amino-5-bromopyridine-2-carboxylate (50 g, 85.7% yield) as a yellow solid. Mass Spectrum (ESI) m/z=244.9 (M+1).

Step B: To a mixture of ethyl 3-amino-5-bromopyridine-2-carboxylate (50 g, 0.2 mol) in 1,4-dioxane (200 mL) was added ammonium hydroxide (500 mL, 3.6 mol) carefully. The reaction was stirred for 48 h at 100° C. Then the reaction was concentrated and purified by silica gel column chromatography (400 g, eluting with DCM/MeOH=10:1) to give 3-amino-5-bromopyridine-2-carboxamide (36 g, 75.0% yield) as a yellow solid. Mass Spectrum (ESI) m/z=215.9 (M+1).

Step C: A mixture of 3-amino-5-bromopyridine-2-carboxamide (18 g, 0.08 mol) and triphosgene (202 g, 0.68 mol) was stirred at 160° C. for 4 h. Then the reaction was cooled to rt and diluted with MeOH (200 mL). The resulting mixture was stirred for 2 h, then filtered. The solid was dried under reduced pressure to give 7-bromopyrido[3,2-d]pyrimidine-2,4-diol (18 g, 87.5% yield) as a yellow solid. Mass Spectrum (ESI) m/z=241.7 (M+1).

Step D: To a mixture of 7-bromopyrido[3,2-d] pyrimidine-2,4-diol (16 g, 66.11 mmol) and DIEA (17.09 g, 132.22 mmol) in toluene (200 mL) stirred under nitrogen was added phosphorus oxychloride (40.55 g, 264.44 mmol) carefully. The reaction mixture was stirred at 110° C. for 4 h. Then the reaction was concentrated and diluted with DCM (400 mL). The solution was quenched with ice/water. The organic layer was washed with sat. NaHCO₃ solution and brine, dried over Na₂SO₄, filtered and the filtrate was concentrated. The crude product was purified by CombiFlash® (120 g, PE/EA=3:1) to give 7-bromo-2,4-dichloropyrido[3,2-d]pyrimidine (8.5 g, 41.2% yield) as a yellow solid. ¹H NMR (400 MHz, CDCl₃) δ 9.13 (d, J=2.1 Hz, 1H), 8.51 (d, J=2.1 Hz, 1H).

Step E: To a mixture of 7-bromo-2,4-dichloropyrido[3,2-d]pyrimidine (500 mg, 1.79 mmol) and triethylamine (398.5 g, 3.94 mmol) in EtOH (10 mL) was added cyclopentyl-methyl-amine (186.4 mg, 1.88 mmol). The reaction mixture was stirred at rt overnight. The mixture was concentrated, and the residue was purified by CombiFlash® (12 g, PE/EA=4:1) to give 7-bromo-2-chloro-N-cyclopentyl-N-methylpyrido[3,2-d]pyrimidin-4-amine (600 mg, 88.3% yield) as a yellow solid. Mass Spectrum (ESI) m/z=341.0 (M+1).

Step F: To a solution of 7-bromo-2-chloro-N-cyclopentyl-N-methylpyrido[3,2-d]pyrimidin-4-amine (400 mg, 1.17 mmol) in THF (5 mL) was added n-butyllithium (0.49 mL, 1.17 mmol, 2.4 M) dropwise at −78° C. under nitrogen. The reaction was stirred for 30 min at −78° C. Then a solution of (3R,4R,5R)-3,4-bis(benzyloxy)-5-[(benzyloxy)methyl]oxolan-2-one (514.1 mg, 1.23 mmol) in THF (5 mL) was added at −78° C. under nitrogen. The reaction was stirred at −78° C. for 2 h, then quenched with sat. aq. NH₄Cl solution and extracted with EA (20 mL×3). The combined organic layers were washed with brine, dried over Na₂SO₄, filtered and the filtrate was concentrated. The crude product was purified by CombiFlash® (12 g, PE/EA=3:1) to give (3R,4R,5R)-3,4-bis(benzyloxy)-5-[(benzyloxy)methyl]-2-{2-chloro-4-[cyclopentyl(methyl)amino]pyrido[3,2-d]pyrimidin-7-yl}oxolan-2-ol (610 mg, 69.2% yield) as a yellow oil. Mass Spectrum (ESI) m/z=681.1 (M+1).

Step G: To a solution of (3R,4R,5R)-3,4-bis(benzyloxy)-5-[(benzyloxy)methyl]-2-{2-chloro-4-[cyclopentyl(methyl)amino]pyrido[3,2-d]pyrimidin-7-yl}oxolan-2-ol (610 mg, 0.89 mmol) in DCM (15 mL) was added boron trifluoride etherate (1088.6 mg, 3.58 mmol, 46.7%) carefully at −78° C. under nitrogen. Then triethylsilane (416.5 mg, 3.58 mmol) was added at −78° C. under nitrogen. The reaction was stirred for 2 h at 20° C. The reaction was quenched with sat. NaHCO₃ solution and extracted with DCM (20 mL×3). The combined organic layers were washed with brine and dried over Na₂SO₄, filtered and the filtrate was concentrated. The crude product was purified by CombiFlash® (12 g, PE/EA=3:1) to give 7-[(2S,3S,4R,5R)-3,4-bis(benzyloxy)-5-[(benzyloxy)methyl]oxolan-2-yl]-2-chloro-N-cyclopentyl-N-methylpyrido[3,2-d]pyrimidin-4-amine (470 mg, 71.5% yield) as a yellow oil. Mass Spectrum (ESI) m/z=665.2 (M+1).

Step H and Step I: 7-[(2S,3S,4R,5R)-3,4-bis(benzyloxy)-5-[(benzyloxy)methyl]oxolan-2-yl]-2-chloro-N-cyclopentyl-N-methylpyrido[3,2-d]pyrimidin-4-amine was converted to the title compound by procedures similar to the ones described in Example S1 Step H and Step I.

¹H NMR (400 MHz, D₂O) δ 8.73 (s, 1H), 8.14 (s, 1H), 5.83 (s, 1H), 4.97 (d, J=6.9 Hz, 1H), 4.35-4.22 (m, 2H), 4.20-4.01 (m, 3H), 3.40 (s, 3H), 2.14 (t, J=19.7 Hz, 2H), 2.03-1.94 (m, 2H), 1.72-1.56 (m, 6H). Mass Spectrum (ESI) m/z=553.1 (M+1).

### Example S42

### Synthesis of (((((2R,3S,4R,5S)-5-(2-chloro-4-(((4-hydroxycyclohexyl)methyl)amino)quinazolin-7-yl)-3,4-dihydroxytetrahydrofuran-2-yl)methoxy)(hydroxy)phosphoryl)methyl)phosphonic acid

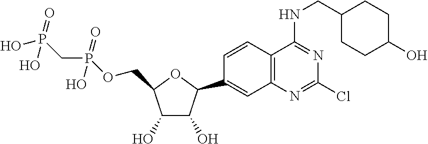 [SMILES: O=P(O)(O)CP(=O)(O)OC[C@H]1O[C@@H](c2ccc3c(NCC4CCC(O)CC4)nc(Cl)nc3c2)[C@H](O)[C@@H]1O]

(((((2R,3S,4R,5S)-5-(2-chloro-4-(((4-hydroxycyclohexyl)methyl)amino)quinazolin-7-yl)-3,4-dihydroxytetrahydrofuran-2-yl)methoxy)(hydroxy)phosphoryl)methyl)phosphonic acid was also by procedures similar to the one described in Example S1, replacing benzylamine in Step G with 4-(aminomethyl)cyclohexan-1-ol.

¹H NMR (400 MHz, D₂O) δ 7.99 (d, J=9.2 Hz, 1H), 7.64-7.53 (m, 2H), 4.94 (d, J=6.9 Hz, 1H), 4.22-4.08 (m, 4H), 3.94-3.81 (m, 2H), 3.41 (d, J=7.0 Hz, 2H), 2.20-2.04 (m, 4H), 1.93-1.84 (m, 2H), 1.80-1.73 (m, 1H), 1.46-1.34 (m, 2H),1.19-1.10 (m, 2H). Mass Spectrum (ESI) m/z=580.0 (M−1).

### Example S43

### Synthesis of (((((2R,3S,4R,5S)-5-(2-chloro-4-((3-hydroxycyclopentyl)amino)quinazolin-7-yl-3,4-dihydroxytetrahydrofuran-2-yl)methoxy)(hydroxy)phosphoryl)methyl)phosphonic acid

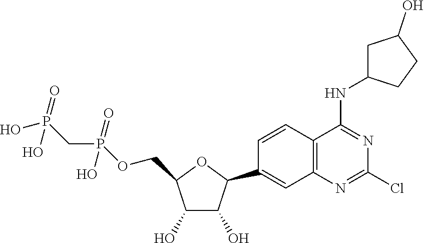 [SMILES: O=P(O)(O)CP(=O)(O)OC[C@H]1O[C@@H](c2ccc3c(NC4CCC(O)C4)nc(Cl)nc3c2)[C@H](O)[C@@H]1O]

(((((2R,3S,4R,5S)-5-(2-chloro-4-((3-hydroxycyclopentyl)amino)quinazolin-7-yl)-3,4-dihydroxytetrahydrofuran-2-yl)methoxy)(hydroxy)phosphoryl)methyl)phosphonic acid was prepared by procedures similar to the one described in Example S1, replacing benzylamine in Step G with 3-aminocyclopentan-1-ol.

¹H NMR (400 MHz, D₂O) δ 8.00 (d, J=6.9 Hz, 1H), 7.63 (d, J=8.0 Hz, 1H), 7.51 (s, 1H), 4.86 (d, J=7.4 Hz, 1H), 4.51-4.42 (m, 1H), 4.36-4.25 (m, 2H), 4.23-4.19 (m, 1H), 4.15-4.02 (m, 3H), 2.41-2.33 (m, 1H), 2.18-2.02 (m, 3H), 1.91-1.73 (m, 3H), 1.66-1.60 (m, 1H). Mass Spectrum (ESI) m/z=554.0 (M+1).

### Example S44

### Synthesis of (((((2R,3S,4R,5S)-5-(2-chloro-4-((3-hydroxycyclobutyl)methyl)amino)quinazolin-7-yl)-3,4-dihydroxytetrahydrofuran-2-yl)methoxy)(hydroxy)phosphoryl)methyl)phosphonic acid

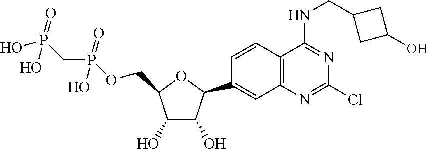 [SMILES: O=P(O)(O)CP(=O)(O)OC[C@H]1O[C@@H](c2ccc3c(NCC4CC(O)C4)nc(Cl)nc3c2)[C@H](O)[C@@H]1O]

(((((2R,3S,4R,5S)-5-(2-chloro-4-(((3-hydroxycyclobutyl)methyl)amino)quinazolin-7-yl)-3,4-dihydroxytetrahydrofuran-2-yl)methoxy)(hydroxy)phosphoryl)methyl)phosphonic acid was prepared by procedures similar to the one described in Example S1, replacing benzylamine in Step G with 3-(aminomethyl)cyclobutan-1-ol.

¹H NMR (400 MHz, D₂O) δ 7.96 (d, J=8.6 Hz, 1H), 7.63 (d, J=8.8 Hz, 1H), 7.48 (s, 1H), 4.85 (d, J=7.1 Hz, 1H), 4.31-4.27 (m, 1H), 4.21-4.19 (m, 1H), 4.14-3.99 (m, 4H), 3.51-3.49 (m, 2H), 2.36-2.32 (m, 2H), 2.12-2.10 (m,1H), 2.01-1.99 (m, 2H), 1.59-1.57 (m, 2H). Mass Spectrum (ESI) m/z=551.9 (M−1).

### Example S45

### Synthesis of (((((2R,3S,4R,5S)-5-(4-(benzylamino)-2-cyclopropylquinazolin-7-yl)-3,4-dihydroxytetrahydrofuran-2-yl)methoxy)(hydroxy)phosphoryl)methyl)phosphonic acid

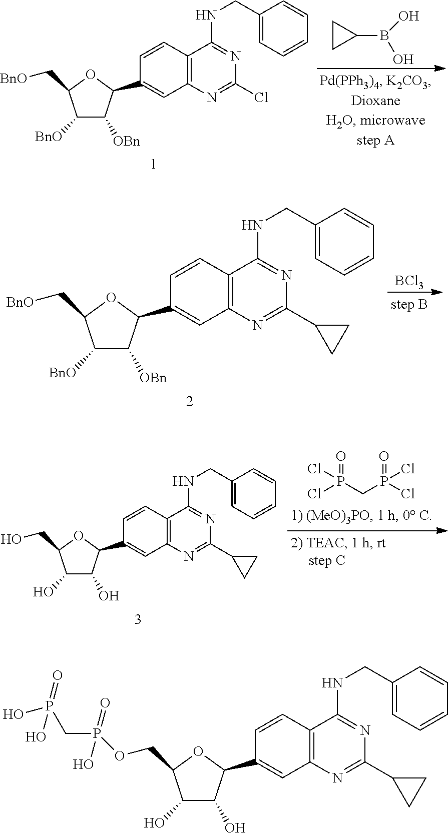

Step A: To a solution of N-benzyl-7-[(2S,3S,4R,5R)-3,4-bis(benzyloxy)-5-[(benzyloxy)methyl]oxolan-2-yl]-2-chloroquinazolin-4-amine (Example S1, Step G, 2.8 g, 4.16 mmol) in dioxane (20 mL) was added K₂CO₃ (1.72 g, 12.48 mmol), cyclopropylboronic acid (430 mg, 5.0 mmol) and tetrakis(triphenylphosphine)palladium (960 mg, 0.83 mmol). The reaction was stirred for 3 h in a microwave reactor. Then water was added, and the mixture was extracted with EA (3×120 mL). The combined organic layers were dried over anhydrous Na₂SO₄, filtered, and the filtrate was concentrated under reduced pressure. The residue was purified by column chromatography on silica gel (PE/EA=5:1) to give N-benzyl-7-[(2S,3S,4R,5R)-3,4-bis(benzyloxy)-5-[(benzyloxy)methyl]oxolan-2-yl]-2-cyclopropylquinazolin-4-amine (1.2 g, 42% yield) as a yellow oil. Mass Spectrum (ESI) m/z=678.1 (M+1).

Step B and Step C: N-benzyl-7-[(2S,3S,4R,5R)-3,4-bis(benzyloxy)-5-[(benzyloxy)methyl]oxolan-2-yl]-2-cyclopropylquinazolin-4-amine was converted to the title compound by procedures similar to the ones described in Example S1 Step H and Step I.

¹H NMR (400 MHz, D₂O) δ 7.93 (d, J=9.1 Hz, 1H), 7.54 (d, J=7.4 Hz, 2H), 7.33 (d, J=7.1 Hz, 2H), 7.27 (t, J=7.4 Hz, 2H), 7.21-7.19 (m, 1H), 4.84 (d, J=6.7 Hz, 1H), 4.63 (s, 2H), 4.32-4.28 (m, 1H), 4.20-4.16 (m, 1H), 4.14-4.06 (m, 3H), 2.03-1.90 (m, 3H), 0.94-0.87 (m, 4H). Mass Spectrum (ESI) m/z=564.1 (M−1).

### Example S46

### Synthesis of [({[(2R,3S,4R,5S)-5-[4-(cyclopentylamino)quinazolin-7-yl]-3,4-dihydroxyoxolan-2-yl]methoxy}(hydroxy)phosphoryl)methyl]phosphonic acid

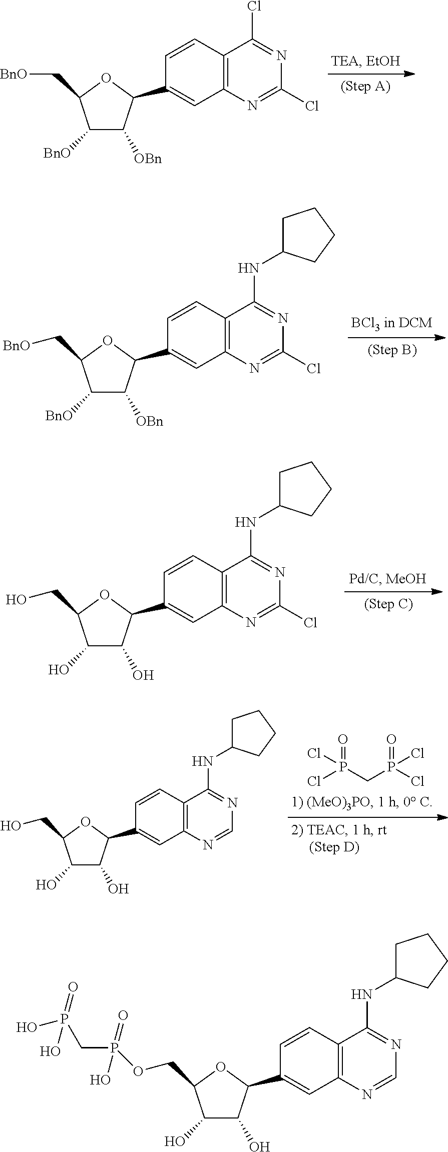 [SMILES: Clc1nc(Cl)c2ccc([C@@H]3O[C@H](COCc4ccccc4)[C@@H](OCc4ccccc4)[C@H]3OCc3ccccc3)cc2n1 ; Clc1nc(NC2CCCC2)c2ccc([C@@H]3O[C@H](COCc4ccccc4)[C@@H](OCc4ccccc4)[C@H]3OCc3ccccc3)cc2n1 ; OC[C@H]1O[C@@H](c2ccc3c(NC4CCCC4)nc(Cl)nc3c2)[C@H](O)[C@@H]1O ; OC[C@H]1O[C@@H](c2ccc3c(NC4CCCC4)ncnc3c2)[C@H](O)[C@@H]1O ; O=P(Cl)(Cl)CP(=O)(Cl)Cl ; O=P(O)(O)CP(=O)(O)OC[C@H]1O[C@@H](c2ccc3c(NC4CCCC4)ncnc3c2)[C@H](O)[C@@H]1O]

Step A: Cyclopentylamine (146 mg, 1.8 mmol) and TEA (251 mg, 2.49 mmol) were added to a solution of 7-[(2S,3S,4R,5R)-3,4-bis(benzyloxy)-5-[(benzyloxy)methyl]oxolan-2-yl]-2,4-dichloroquinazoline (1 g, 1.66 mmol) in EtOH (15 mL). The reaction mixture was stirred at rt for 1 h. The solvent was removed under vacuo and the crude residue was purified with column chromatography on silica gel (PE/EA=2:1) to give 7-[(2S,3S,4R,5R)-3,4-bis(benzyloxy)-5-[(benzyloxy)methyl]oxolan-2-yl]-2-chloro-N-cyclopentylquinazolin-4-amine (1 g, 92% yield) as a white solid. Mass Spectrum (ESI) m/z=650.2 (M+1).

Step B: To a solution of 7-[(2S,3S,4R,5R)-3,4-bis(benzyloxy)-5-[(benzyloxy)methyl]oxolan-2-yl]-2-chloro-N-cyclopentylquinazolin-4-amine (900 mg, 1.38 mol) in DCM (10 mL) was added a solution of BCl₃ in DCM (1 M, 13.8 mL, 13.8 mmol) dropwise at −78° C. under a nitrogen atmosphere. The mixture was stirred at the same temperature for 2 h, then quenched with DCM/MeOH (1:1, 20 mL). The reaction mixture was allowed to warm to rt, then it was neutralized with NH₃ in methanol (10%, 40 mL) and concentrated. The residue was purified by column chromatography on silica gel (DCM/MeOH=10:1) to give (2S,3R,4S,5R)-2-[2-chloro-4-(cyclopentylamino)quinazolin-7-yl]-5-(hydroxymethyl)oxolane-3,4-diol (400 mg, 74% yield) as a white solid. Mass Spectrum (ESI) m/z=378.1 (M+1).

Step C: Pd/C (126 mg) was added to a solution of (2S,3R,4S,5R)-2-[2-chloro-4-(cyclopentylamino)quinazolin-7-yl]-5-(hydroxymethyl)oxolane-3,4-diol (420 mg, 1.11 mmol) in MeOH (10 mL). The reaction mixture was stirred at rt for 3 h under a H₂ atmosphere. The catalyst was filtered off, and the filtrate was concentrated. The residue was purified by column chromatography on silica gel (DCM/MeOH=10:1) to give (2S,3R,4S,5R)-2-[4-(cyclopentylamino)quinazolin-7-yl]-5-(hydroxymethyl)oxolane-3,4-diol (310 mg, 79% yield) as a white solid. Mass Spectrum (ESI) m/z=346.2 (M+1).

Step D: (2S,3R,4S,5R)-2-[4-(cyclopentylamino)quinazolin-7-yl]-5-(hydroxymethyl)oxolane-3,4-diol was converted to the title compound by a procedures similar to the one described in Example S1 Step I.

¹H NMR (400 MHz, D₂O) δ 8.29 (s, 1H), 8.02 (d, J=7.9 Hz, 1H), 7.75-7.58 (m, 2H), 4.89 (d, J=6.8 Hz, 1H), 4.43-4.29 (m, 2H), 4.23-4.05 (m, 4H), 2.09-1.92 (m, 4H), 1.74-1.52 (m, 6H). Mass Spectrum (ESI) m/z=502.1 (M−1).

### BIOLOGICAL EXAMPLES

A variety of assays can be used to evaluate inhibition of compounds for CD73. Compounds of the present disclosure display inhibition of CD73 in the following assays.

### Example B1

### CD73 Enzyme Assay

Soluble recombinant CD73 catalyzes the conversion of adenosine monophosphate (AMP) to adenosine and inorganic phosphate. The phosphate detection reagent, PiColorLock™ (Innova Bioscience, Cat #303-0125) is based on the change in absorbance of the dye malachite green in the presence of inorganic phosphate (Pi) and this property can be exploited to measure any enzyme that generates Pi. Recombinant Human 5′-Nucleotidase (CD73) (R&D #5795-EN, CHO derived CD73 (Trp27-Lys547), with a C-terminal 6-His tag) was used in the enzymatic assay. This assay was run in a 384-well plate format (Corning® NBS™ 384 well plates, Cat #3640). The basic assay procedure involves two steps: 1) Enzyme reaction: The CD73 enzyme (R&D #5795-EN) is incubated in the presence or absence of compounds. AMP (sigma, cat #01930) is added to start the kinase reaction. 2) Detection step: “Gold mix” is added to the assay system, then stabilizers are added. After incubation the absorbance of the solution is read at OD 635 nm. The recorded OD signal is proportional to the enzyme activity.

Briefly, 25 μl human CD73 (0.5 nM final concentration) in the enzymatic buffer solutions (20 mM Tris, 25 mM NaCl, 1 mM MgCl₂, pH 7.5, 0.005% Tween-20) were mixed with various concentrations of the test compound (dissolved in 100% DMSO). These solutions were incubated for 15 min at 25° C., and subsequently 25 μl AMP (30 μM final concentration) was added to start the reaction. The final reaction mixture of enzyme-substrate-compound was incubated for 20 min at 37° C. Meanwhile, “Gold mix” was prepared shortly before use by adding 1/100 vol. of accelerator to the PiColorLock™ Gold reagent. 12 μL/well of the “Gold mix” was added to assay plate containing 50 μL enzyme reaction buffer and incubated at 25° C. for 5 min. 5 μL/well stabilizer was added to the assay plate and incubated at 25° C. for 30 min. The absorbance of the well solutions was measured at 635 nm on a Spark 10M instrument (TECAN).

The percent (%) inhibition at each concentration of a compound was calculated relative to the OD value in the Max and Min control wells contained within each assay plate. The Max control wells contained enzyme and substrate as 0% inhibition, and the Min control wells only contained substrate without enzyme as 100% inhibition. The concentrations and percent inhibition values for a test compound are plotted and the concentration of the compound required to achieve 50% inhibition (IC₅₀) was determined with a four-parameter logistic dose response equation. Results for certain compounds are provided in the Table 2 below.

TABLE 2

| Compound | | Compound | | Compound | |
|---|---|---|---|---|---|
| No. | Potency | No. | Potency | No. | Potency |
| 1 | b | 2 | b | 3 | b |
| 4 | a | 5 | b | 6 | b |
| 7 | b | 8 | a | 9 | b |
| 10 | b | 11 | d | 12 | b |
| 13 | c | 14 | d | 15 | c |
| 16 | d | 17 | b | 18 | b |
| 19 | c | 20 | c | 21 | b |
| 22 | a | 23 | a | 24 | a |
| 25 | a | 26 | b | 27 | c |
| 28 | a | 29 | b | 30 | c |
| 31 | a | 32 | c | 33 | c |
| 34 | d | 35 | b | 36 | b |
| 37 | b | 38 | b | 39 | b |
| 40 | b | 41 | b | 42 | b |
| 43 | c | 44 | b | 45 | b |
| 46 | a | | | | |

“a” means an IC₅₀ of <10 nM;
“b” means an IC₅₀ of 10-99 nM;
“c” means an IC₅₀ of 100-999 nM; and
“d” means IC₅₀ of >1000 nM

### Example B2

### CD73 Cellular Assay

Cell surface CD73 catalyzes the conversion of adenosine monophosphate (AMP) to adenosine and inorganic phosphate. U87 MG human glioblastoma cells express high level of CD73. Cells are treated with compounds in a 96-well assay plate and supernatants are collected into a 384-well detection plate. The concentration of inorganic phosphate (Pi) in the supernatants is determined using the phosphate detection reagent, PiColorLock™ (Innova Bioscience, Cat #303-0125) following the manufacturer's instructions.

Briefly, on the day of the assay, U87 MG cells were harvested and resuspended in assay buffer which consisted of 20 mM HEPES, pH=7.4, 137 mM NaCl, 5.4 mM KCl, 1.3 mM CaCl₂, 4.2 mM NaHCO₃ and 0.1% glucose. To test the effect of compounds on cellular CD73 enzymatic activity, 500 nL/well of compounds dissolved in DMSO were added to a 96-well TC-treated microplate (Corning #3599). Next, 80 μL/well of U87 MG cells in assay buffer were added to assay plate. After 30 minutes of incubation in an atmosphere of 5% CO₂ at 37° C., 20 μL/well of 150 μM AMP (Adenosine 5′-monophosphate monohydrate, Sigma, Cat #01930) in assay buffer was added to assay plate. Final assay conditions consisted of 5000 cells per well in 0.5% DMSO and 30 μM AMP substrate. After 50 minutes of incubation in an atmosphere of 5% CO₂ at 37° C., 50 μL/well of supernatant was transferred to the 384-well detection plate (Corning® NBSTM 384 well plates, Cat #3640). Meanwhile “Gold mix” was prepared shortly before use by adding 1/100 volume of accelerator to the PiColorLock™ Gold reagent. 12 μL/well of the “Gold mix” was added to detection plate containing 50 μL/well of supernatant and incubated at 25° C. for 5 minutes. 5 μL/well stabilizer was added to the detection plate and incubated at 25° C. for 30 minutes. The absorbance at 635 nm was measured on a Spark 10M instrument (TECAN).

The percent (%) inhibition at each concentration of a compound was calculated relative to the OD value in the Max and Min control wells contained within each assay plate. The Max control wells contained cells and substrate as 0% inhibition, and the Min control wells only contained cells as 100% inhibition. The concentrations and percent inhibition values for a test compound are plotted and the concentration of the compound required to achieve 50% inhibition (IC50) was determined with a four-parameter logistic dose response equation. Results for certain compounds are provided in the Table 3 below.

TABLE 3

| Compound | | Compound | | Compound | |
|---|---|---|---|---|---|
| No. | Potency | No. | Potency | No. | Potency |
| 1 | a | 2 | a | 3 | a |
| 4 | a | 5 | a | 6 | a |
| 7 | a | 8 | a | 9 | a |
| 10 | a | 12 | a | 13 | b |
| 17 | a | 18 | a | 22 | a |
| 23 | a | 25 | a | 28 | c |
| 31 | a | 46 | d | | |

“a” means an IC₅₀ of <10 nM;
“b” means an IC₅₀ of 10-99 nM;
“c” means an IC₅₀ of 100-999 nM; and
“d” means IC₅₀ of >1000 nM

All references throughout, such as publications, patents, patent applications and published patent applications, are incorporated herein by reference in their entireties.

1. A compound of formula (I): 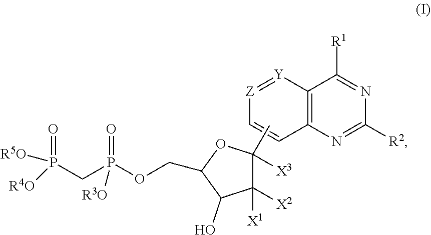
or a stereoisomer, tautomer, prodrug, or a pharmaceutically acceptable salt of any of the foregoing, wherein:
X¹, X², and X³ are each independently H, —CN, C₁₋₆ alkyl, —OR′, or halogen, wherein R′ is H, C₁₋₆ alkyl, C₃₋₁₂ cycloalkyl, 3- to 12-membered heterocyclyl, 5- to 10-membered heteroaryl, or C₆₋₁₄ aryl;
Y is —CR^{Y}— or N, wherein R^{Y} is H, C₁₋₆ alkyl, or halogen;
Z is —CR^{Z}— or N, wherein R^{Z} is H, C₁₋₆ alkyl, or halogen;
R¹ is —NR^{1a}R^{1b} or —OR^{1a}, wherein R^{1a} and R^{1b} are each independently H, C₁₋₆ alkyl, C₃₋₁₂ cycloalkyl, 3- to 12-membered heterocyclyl, 5- to 10-membered heteroaryl, or C₆₋₁₄ aryl, wherein the C₁₋₆ alkyl, C₃₋₁₂ cycloalkyl, 3- to 12-membered heterocyclyl, 5- to 10-membered heteroaryl, and C₆₋₁₄ aryl are each independently optionally substituted with R⁶, or
R^{1a} and R^{1b} are taken together with the nitrogen atom to which they attach to form a 3- to 12-membered heterocyclyl, which is optionally substituted with R⁶;
R² is H, C₁₋₆ alkyl, C₂₋₆ alkenyl, C₂₋₆ alkynyl, halogen, —CN, —OR^{2a}, —SR^{2a}, —NR^{2a}R^{2b}, —OC(O)R^{2a}, —NR^{2a}C(O)R^{2b}, —NR^{2a}C(O)OR^{2b}, —NR^{2a}S(O)R^{2b}, —NR^{2a}S(O)₂R^{2b}, —C(O)NR^{2a}R^{2b}, —C(O)NR^{2a}S(O)₂R^{2b}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently optionally substituted with R⁷, and wherein:
R^{2a} and R^{2b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{2a} and R^{2b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
R³, R⁴, and R⁵ are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl;
each R⁶ is independently oxo, C₁₋₆ alkyl, C₂₋₆ alkenyl, C₂₋₆ alkynyl, halogen, —CN, —OR^{6a}, —SR^{6a}, —NR^{6a}R^{6b}, —NO₂, —C═NH(OR^{6a}), —C(O)R^{6a}, —OC(O)R^{6a}, —C(O)OR^{6a}, —C(O)NR^{6a}R^{6b}, —OC(O)NR^{6a}R^{6b}, —NR^{6a}C(O)R^{6b}, —NR^{6a}C(O)OR^{6b}, —S(O)R^{6a}, —S(O)₂R^{6a}, —NR^{6a}S(O)R^{6b}, —C(O)NR^{6a}S(O)R^{6b}, —NR^{6a}S(O)₂R^{6b}, —C(O)NR^{6a}S(O)₂R^{6b}, S(O)NR^{6a}R^{6b}, —S(O)₂NR^{6a}R^{6b}, —P(O)(^{6a}) (OR^{6b}), C₃₋₆ cycloalkyl, 3- to 12-membered heterocyclyl, 5- to 10-membered heteroaryl, or C₆₋₁₄ aryl, wherein the C3- to 12-membered heterocyclyl, 5- to 10-membered heteroaryl, and C₆₋₁₄ aryl are each independently optionally substituted with C₁₋₆ alkyl, C₂₋₆ alkenyl, C₂₋₆ alkynyl, halogen, hydroxyl, C₁₋₆ alkoxy, or —CN, and wherein:
R^{6a} and R^{6b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{6a} and R^{6b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
each R⁷ is independently oxo, C₁₋₆ alkyl, C₂₋₆ alkenyl, C₂₋₆ alkynyl, halogen, —CN, —SR^{7a}, —NR^{7a}R^{7b}, —NO₂, —C═NH(OR^{7a}, —C(O)R^{7a}, —OC(O)R^{7a}, —C(O)OR^{7a}, —C(O)NR^{7a}R^{7b}, —OC(O)NR^{7a}R^{7b}, —NR^{7a}C(O)R^{7b}, —NR^{7a}C(O)R^{7b}, —S(O)R^{7a}, —S(O)₂R^{7a}, —NR^{7a}S(O)R^{7b}, —C(O)NR^{7a}S(O)R^{7b}, —NR^{7a}S(O)₂R^{7b}, —C(O)NR^{7a}S(O)₂R^{7b}, —S(O)NR^{7a}R^{7b}, —S(O)₂NR^{7a}R^{7b}, —P(O)(OR^{7a}) (OR^{7b}), C₃₋₆ cycloalkyl, 3- to 12-membered heterocyclyl, 5- to 10-membered heteroaryl, or C₆₋₁₄ aryl, wherein:
R^{7a} and R^{7b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{7a} and R^{7b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN.

2. The compound of claim 1, or a stereoisomer, tautomer, prodrug, or a pharmaceutically acceptable salt of any of the foregoing, wherein the compound is of formula (II):

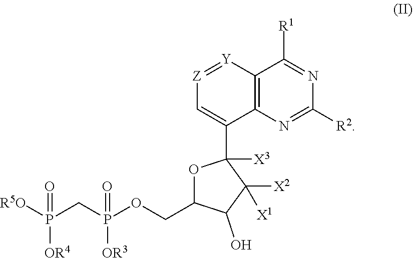

3. The compound of claim 1, or a stereoisomer, tautomer, prodrug, or a pharmaceutically acceptable salt of any of the foregoing, wherein the compound is of formula (III):

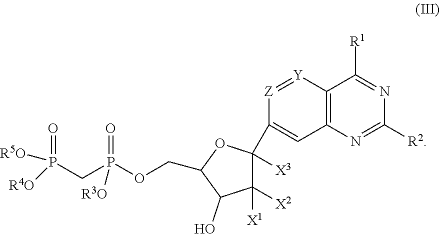

4. The compound of claim 1, or a stereoisomer, tautomer, prodrug, or a pharmaceutically acceptable salt of any of the foregoing, wherein Y is —CR^{Y}—.

5. The compound of claim 1, or a stereoisomer, tautomer, prodrug, or a pharmaceutically acceptable salt of any of the foregoing, wherein R^{Y} is H.

6. The compound of claim 1, or a stereoisomer, tautomer, prodrug, or a pharmaceutically acceptable salt of any of the foregoing, wherein Z is —CR^{Z}—.

7. The compound of claim 1, or a stereoisomer, tautomer, prodrug, or a pharmaceutically acceptable salt of any of the foregoing, wherein R^{Z} is H or halogen.

8. The compound of claim 1, or a stereoisomer, tautomer, prodrug, or a pharmaceutically acceptable salt of any of the foregoing, wherein X¹ is H or —OH.

9. The compound of claim 1, or a stereoisomer, tautomer, prodrug, or a pharmaceutically acceptable salt of any of the foregoing, wherein X² is H, halogen, or C₁₋₆ alkyl.

10. The compound of claim 1, or a stereoisomer, tautomer, prodrug, or a pharmaceutically acceptable salt of any of the foregoing, wherein X³ is H or —CN.

11. The compound of claim 1, or a stereoisomer, tautomer, prodrug, or a pharmaceutically acceptable salt of any of the foregoing, wherein R¹ is —NR^{1a}R^{1b}.

12. The compound of claim 1, or a stereoisomer, tautomer, prodrug, or a pharmaceutically acceptable salt of any of the foregoing, wherein R¹ is —OR^{1a}.

13. The compound of claim 1, or a stereoisomer, tautomer, prodrug, or a pharmaceutically acceptable salt of any of the foregoing, wherein R^{1a} is C₁₋₆ alkyl, C₃₋₁₂ cycloalkyl, or 3- to 12-membered heterocyclyl, each of which is independently optionally substituted with R⁶.

14. The compound of claim 1, or a stereoisomer, tautomer, prodrug, or a pharmaceutically acceptable salt of any of the foregoing, wherein R⁶ is —OR^{6a}, C₃₋₆ cycloalkyl, 3- to 12-membered heterocyclyl, or C₆₋₁₄ aryl, wherein the C₃₋₆ cycloalkyl, 3- to 12-membered heterocyclyl, and C₆₋₁₄ aryl of R⁶ are each independently optionally substituted with halogen or hydroxyl, and wherein R^{6a} is H or C₁₋₆ alkyl.

15. The compound of claim 1, or a stereoisomer, tautomer, prodrug, or a pharmaceutically acceptable salt of any of the foregoing, wherein R^{1a} is

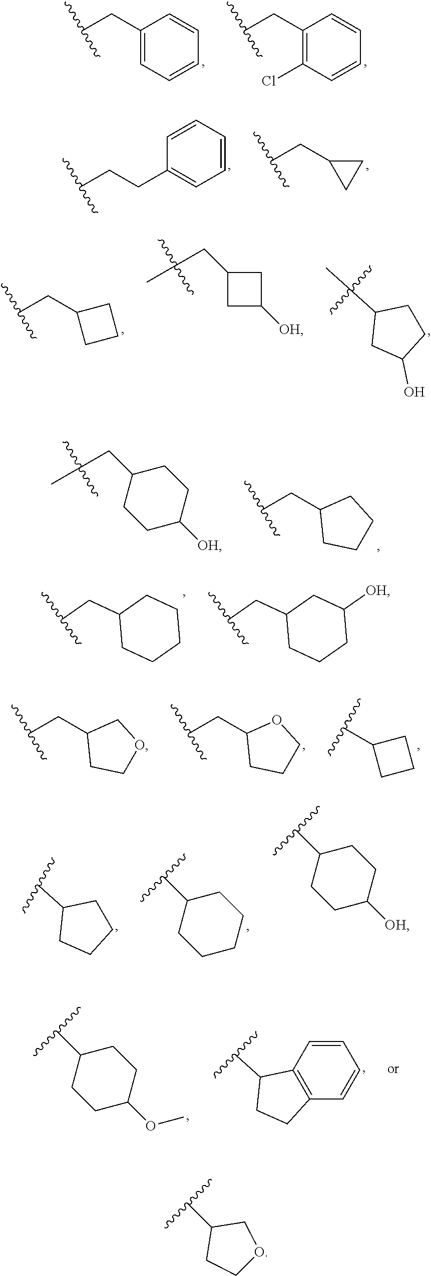

16. The compound of claim 1, or a stereoisomer, tautomer, prodrug, or a pharmaceutically acceptable salt of any of the foregoing, wherein R^{1b} is H or C₁₋₆ alkyl.

17. The compound of claim 1, or a stereoisomer, tautomer, prodrug, or a pharmaceutically acceptable salt of any of the foregoing, wherein R^{1a} and R^{1b} are taken together with the nitrogen atom to which they attach to form a 3- to 12-membered heterocyclyl, which is optionally substituted with R⁶.

18. The compound of claim 17, or a stereoisomer, tautomer, prodrug, or a pharmaceutically acceptable salt of any of the foregoing, wherein R^{1a} and R^{1b} are taken together with the nitrogen atom to which they attach to form

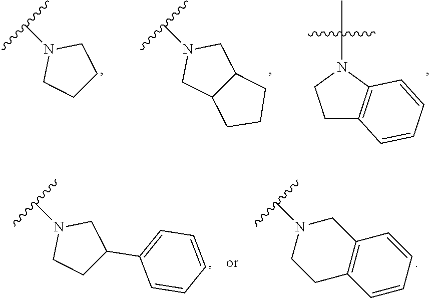 [SMILES: *N1CCCC1 ; *N1CC2CCCC2C1 ; *N1CCc2ccccc21 ; *N1CCC(c2ccccc2)C1 ; *N1CCc2ccccc2C1]

19. The compound of claim 1, or a stereoisomer, tautomer, prodrug, or a pharmaceutically acceptable salt of any of the foregoing, wherein R² is H, halogen, C₁₋₆ alkyl, C₃₋₆ cycloalkyl, or C₂₋₆ alkenyl, wherein the C₁₋₆ alkyl, C₃₋₆ cycloalkyl, and C₂₋₆ alkenyl are each independently optionally substituted with R⁷.

20. The compound of claim 1, or a stereoisomer, tautomer, prodrug, or a pharmaceutically acceptable salt of any of the foregoing, wherein R² is H, chloro, —CH₃, —CH₂CH₃, cyclopropyl, or —CH═CH₂.

21. The compound of claim 1, or a stereoisomer, tautomer, prodrug, or a pharmaceutically acceptable salt of any of the foregoing, wherein R³ is H.

22. The compound of claim 1, or a stereoisomer, tautomer, prodrug, or a pharmaceutically acceptable salt of any of the foregoing, wherein R⁴ is H.

23. The compound of claim 1, or a stereoisomer, tautomer, prodrug, or a pharmaceutically acceptable salt of any of the foregoing, wherein R⁵ is H.

24. A compound selected from the group consisting of the compounds in Table 1, or a stereoisomer, tautomer, prodrug, or a pharmaceutically acceptable salt of any of the foregoing.

25. A pharmaceutical composition comprising at least one compound according to claim 1, or a stereoisomer, tautomer, prodrug, or a pharmaceutically acceptable salt of any of the foregoing, optionally further comprising a pharmaceutically acceptable excipient.

26. A kit comprising at least one compound according to claim 1, or a stereoisomer, tautomer, prodrug, or a pharmaceutically acceptable salt of any of the foregoing,

27. A method of treating a disease mediated by CD73 in an individual in need thereof, comprising administering to the individual a therapeutically effective amount of a compound according to claim 1, or a stereoisomer, tautomer, prodrug, or a pharmaceutically acceptable salt of any of the foregoing.

28. The method of claim 27, wherein the disease is cancer.

29. A method of inhibiting CD73, comprising contacting CD73 with a compound according to claim 1, or a stereoisomer, tautomer, prodrug, or a pharmaceutically acceptable salt of any of the foregoing.

30. (canceled)
